SEQUENCE LISTING (251 sequences)

SEQ ID NO: 1 - protein, 6 aa, synthetic construct
GKXXKG

SEQ ID NO: 2 - protein, 6 aa, synthetic construct
GKAQKG

SEQ ID NO: 3 - protein, 6 aa, synthetic construct
GKHLKG

SEQ ID NO: 4 - protein, 6 aa, synthetic construct
GKQVKG

SEQ ID NO: 5 - protein, 6 aa, synthetic construct
GKLTKG

SEQ ID NO: 6 - protein, 6 aa, synthetic construct
GKDTKG

SEQ ID NO: 7 - protein, 6 aa, synthetic construct
GKNTKG

SEQ ID NO: 8 - protein, 6 aa, synthetic construct
GKDIKG

SEQ ID NO: 9 - protein, 6 aa, synthetic construct
GKRHKG

SEQ ID NO: 10 - protein, 6 aa, synthetic construct
GKRTKG

SEQ ID NO: 11 - protein, 6 aa, synthetic construct
GKNCKG

SEQ ID NO: 12 - protein, 6 aa, synthetic construct
GKHTKG

SEQ ID NO: 13 - protein, 6 aa, synthetic construct
GKQPKG

SEQ ID NO: 14 - protein, 6 aa, synthetic construct
GKREKG

SEQ ID NO: 15 - protein, 6 aa, synthetic construct
GKMPKG

SEQ ID NO: 16 - protein, 6 aa, synthetic construct
GKRRKG

SEQ ID NO: 17 - protein, 6 aa, synthetic construct
GKEMKG

SEQ ID NO: 18 - protein, 6 aa, synthetic construct
GKNIKG

SEQ ID NO: 19 - protein, 6 aa, synthetic construct
GKIPKG

SEQ ID NO: 20 - protein, 6 aa, synthetic construct
GKAEKG

SEQ ID NO: 21 - protein, 6 aa, synthetic construct
GKCEKG

SEQ ID NO: 22 - protein, 6 aa, synthetic construct
GKRMKG

SEQ ID NO: 23 - protein, 6 aa, synthetic construct
GKEPKG

SEQ ID NO: 24 - protein, 6 aa, synthetic construct
GKASKG

SEQ ID NO: 25 - protein, 6 aa, synthetic construct
GKPSKG

SEQ ID NO: 26 - protein, 6 aa, synthetic construct
GKDLKG

SEQ ID NO: 27 - protein, 6 aa, synthetic construct
GKSVKG

SEQ ID NO: 28 - protein, 6 aa, synthetic construct
GKRHKG

SEQ ID NO: 29 - protein, 6 aa, synthetic construct
GKDTKG

SEQ ID NO: 30 - protein, 6 aa, synthetic construct
GKMVKG

SEQ ID NO: 31 - protein, 6 aa, synthetic construct
GKGVKG

SEQ ID NO: 32 - protein, 6 aa, synthetic construct
GKDIKG

SEQ ID NO: 33 - protein, 6 aa, synthetic construct
GKHTKG

SEQ ID NO: 34 - protein, 6 aa, synthetic construct
GKAPKG

SEQ ID NO: 35 - protein, 6 aa, synthetic construct
GKEPKG

SEQ ID NO: 36 - protein, 6 aa, synthetic construct
GKNIKG

SEQ ID NO: 37 - protein, 6 aa, synthetic construct
GKCEKG

SEQ ID NO: 38 - protein, 6 aa, synthetic construct
GKRPKG

SEQ ID NO: 39 - protein, 6 aa, synthetic construct
GKDQKG

SEQ ID NO: 40 - protein, 6 aa, synthetic construct
GKLSKG

SEQ ID NO: 41 - protein, 6 aa, synthetic construct
GKNQKG

SEQ ID NO: 42 - protein, 6 aa, synthetic construct
GKRRKG

SEQ ID NO: 43 - protein, 6 aa, synthetic construct
GKIPKG

SEQ ID NO: 44 - protein, 6 aa, synthetic construct
GKCGKG

SEQ ID NO: 45 - protein, 6 aa, synthetic construct
GKILKG

SEQ ID NO: 46 - protein, 6 aa, synthetic construct
GKLMKG

SEQ ID NO: 47 - protein, 6 aa, synthetic construct
GKCMKG

SEQ ID NO: 48 - protein, 6 aa, synthetic construct
GKIMKG

SEQ ID NO: 49 - protein, 6 aa, synthetic construct
GKMMKG

SEQ ID NO: 50 - protein, 6 aa, synthetic construct
GKIIKG

SEQ ID NO: 51 - protein, 6 aa, synthetic construct
GKCHKG

SEQ ID NO: 52 - protein, 6 aa, synthetic construct
GKNIKG

SEQ ID NO: 53 - protein, 6 aa, synthetic construct
GKRSKG

SEQ ID NO: 54 - protein, 6 aa, synthetic construct
GKDLKG

SEQ ID NO: 55 - protein, 6 aa, synthetic construct
GKRQKG

SEQ ID NO: 56 - protein, 6 aa, synthetic construct
GKRRKG

SEQ ID NO: 57 - protein, 6 aa, synthetic construct
GKDEKG

SEQ ID NO: 58 - protein, 6 aa, synthetic construct
GKRDKG

SEQ ID NO: 59 - protein, 6 aa, synthetic construct
GKADKG

SEQ ID NO: 60 - protein, 6 aa, synthetic construct
GKNDKG

SEQ ID NO: 61 - protein, 6 aa, synthetic construct
GKDDKG

SEQ ID NO: 62 - protein, 6 aa, synthetic construct
GKRMKG

SEQ ID NO: 63 - protein, 6 aa, synthetic construct
GKRNKG

SEQ ID NO: 64 - protein, 6 aa, synthetic construct
GKRQKG

SEQ ID NO: 65 - protein, 6 aa, synthetic construct
GKRGKG

SEQ ID NO: 66 - protein, 6 aa, synthetic construct
GKRMKG

SEQ ID NO: 67 - protein, 6 aa, synthetic construct
GKRLKG

SEQ ID NO: 68 - protein, 6 aa, synthetic construct
GKRPKG

SEQ ID NO: 69 - protein, 6 aa, synthetic construct
GKRRKG

SEQ ID NO: 70 - protein, 6 aa, synthetic construct
GKRCKG

SEQ ID NO: 71 - protein, 6 aa, synthetic construct
GKREKG

SEQ ID NO: 72 - protein, 6 aa, synthetic construct
GKRHKG

SEQ ID NO: 73 - protein, 6 aa, synthetic construct
GKRIKG

SEQ ID NO: 74 - protein, 6 aa, synthetic construct
GKGGKG

SEQ ID NO: 75 - protein, 6 aa, synthetic construct
GKATKG

SEQ ID NO: 76 - protein, 6 aa, synthetic construct
GKSTKG

SEQ ID NO: 77 - protein, 6 aa, synthetic construct
GKGSKG

SEQ ID NO: 78 - protein, 6 aa, synthetic construct
GKASKG

SEQ ID NO: 79 - protein, 6 aa, synthetic construct
GKADKG

SEQ ID NO: 80 - protein, 6 aa, synthetic construct
GKSSKG

SEQ ID NO: 81 - protein, 6 aa, synthetic construct
GKGTKG

SEQ ID NO: 82 - protein, 6 aa, synthetic construct
GKTTKG

SEQ ID NO: 83 - protein, 6 aa, synthetic construct
GKGVKG

SEQ ID NO: 84 - protein, 6 aa, synthetic construct
GKGGKG

SEQ ID NO: 85 - protein, 6 aa, synthetic construct
GKATKG

SEQ ID NO: 86 - protein, 6 aa, synthetic construct
GKSTKG

SEQ ID NO: 87 - protein, 6 aa, synthetic construct
GKGSKG

SEQ ID NO: 88 - protein, 6 aa, synthetic construct
GKASKG

SEQ ID NO: 89 - protein, 6 aa, synthetic construct
GKADKG

SEQ ID NO: 90 - protein, 6 aa, synthetic construct
GKSSKG

SEQ ID NO: 91 - protein, 6 aa, synthetic construct
GKGTKG

SEQ ID NO: 92 - protein, 6 aa, synthetic construct
GKTTKG

SEQ ID NO: 93 - protein, 6 aa, synthetic construct
GKGVKG

SEQ ID NO: 94 - protein, 6 aa, synthetic construct
GKGGKG

SEQ ID NO: 95 - protein, 6 aa, synthetic construct
GKATKG

SEQ ID NO: 96 - protein, 6 aa, synthetic construct
GKSTKG

SEQ ID NO: 97 - protein, 6 aa, synthetic construct
GKGSKG

SEQ ID NO: 98 - protein, 6 aa, synthetic construct
GKASKG

SEQ ID NO: 99 - protein, 6 aa, synthetic construct
GKADKG

SEQ ID NO: 100 - protein, 6 aa, synthetic construct
GKSSKG

SEQ ID NO: 101 - protein, 6 aa, synthetic construct
GKGTKG

SEQ ID NO: 102 - protein, 6 aa, synthetic construct
GKTTKG

SEQ ID NO: 103 - protein, 6 aa, synthetic construct
GKGVKG

SEQ ID NO: 104 - protein, 6 aa, synthetic construct
GKXKXG

SEQ ID NO: 105 - protein, 6 aa, synthetic construct
GKPKQG

SEQ ID NO: 106 - protein, 6 aa, synthetic construct
GKPKQG

SEQ ID NO: 107 - protein, 6 aa, synthetic construct
GKLKDG

SEQ ID NO: 108 - protein, 6 aa, synthetic construct
GKIKQG

SEQ ID NO: 109 - protein, 6 aa, synthetic construct
GKHKDG

SEQ ID NO: 110 - protein, 6 aa, synthetic construct
GKPKDG

SEQ ID NO: 111 - protein, 6 aa, synthetic construct
GKPKEG

SEQ ID NO: 112 - protein, 6 aa, synthetic construct
GKGKDG

SEQ ID NO: 113 - protein, 6 aa, synthetic construct
GKEKMG

SEQ ID NO: 114 - protein, 6 aa, synthetic construct
GKQKDG

SEQ ID NO: 115 - protein, 6 aa, synthetic construct
GKPKQG

SEQ ID NO: 116 - protein, 6 aa, synthetic construct
GKCKHG

SEQ ID NO: 117 - protein, 6 aa, synthetic construct
GKHKHG

SEQ ID NO: 118 - protein, 6 aa, synthetic construct
GKMKHG

SEQ ID NO: 119 - protein, 6 aa, synthetic construct
GKVKHG

SEQ ID NO: 120 - protein, 6 aa, synthetic construct
GKMKMG

SEQ ID NO: 121 - protein, 6 aa, synthetic construct
GKIKHG

SEQ ID NO: 122 - protein, 6 aa, synthetic construct
GKMKAG

SEQ ID NO: 123 - protein, 6 aa, synthetic construct
GKPKHG

SEQ ID NO: 124 - protein, 6 aa, synthetic construct
GKQKLG

SEQ ID NO: 125 - protein, 6 aa, synthetic construct
GKGKCG

SEQ ID NO: 126 - protein, 6 aa, synthetic construct
GKGKMG

SEQ ID NO: 127 - protein, 6 aa, synthetic construct
GKTKRG

SEQ ID NO: 128 - protein, 6 aa, synthetic construct
GKDKNG

SEQ ID NO: 129 - protein, 6 aa, synthetic construct
GKTKDG

SEQ ID NO: 130 - protein, 6 aa, synthetic construct
GKDKDG

SEQ ID NO: 131 - protein, 6 aa, synthetic construct
GKSKCG

SEQ ID NO: 132 - protein, 6 aa, synthetic construct
GKDKRG

SEQ ID NO: 133 - protein, 6 aa, synthetic construct
GKEKRG

SEQ ID NO: 134 - protein, 6 aa, synthetic construct
GKEKHG

SEQ ID NO: 135 - protein, 6 aa, synthetic construct
GKMKLG

SEQ ID NO: 136 - protein, 6 aa, synthetic construct
GKDKHG

SEQ ID NO: 137 - protein, 6 aa, synthetic construct
GKQKMG

SEQ ID NO: 138 - protein, 6 aa, synthetic construct
GKQKHG

SEQ ID NO: 139 - protein, 6 aa, synthetic construct
GKQKVG

SEQ ID NO: 140 - protein, 6 aa, synthetic construct
GKGKPG

SEQ ID NO: 141 - protein, 6 aa, synthetic construct
GKGKIG

SEQ ID NO: 142 - protein, 6 aa, synthetic construct
GKGKEG

SEQ ID NO: 143 - protein, 6 aa, synthetic construct
GKGKQG

SEQ ID NO: 144 - protein, 6 aa, synthetic construct
GKGKRG

SEQ ID NO: 145 - protein, 6 aa, synthetic construct
GKGKVG

SEQ ID NO: 146 - protein, 6 aa, synthetic construct
GKGTDG

SEQ ID NO: 147 - protein, 6 aa, synthetic construct
GKGKSG

SEQ ID NO: 148 - protein, 6 aa, synthetic construct
GKQKAG

SEQ ID NO: 149 - protein, 6 aa, synthetic construct
GKCKPG

SEQ ID NO: 150 - protein, 6 aa, synthetic construct
GKPKIG

SEQ ID NO: 151 - protein, 6 aa, synthetic construct
GKPKPG

SEQ ID NO: 152 - protein, 6 aa, synthetic construct
GKSKVG

SEQ ID NO: 153 - protein, 6 aa, synthetic construct
GKMKVG

SEQ ID NO: 154 - protein, 6 aa, synthetic construct
GKTKIG

SEQ ID NO: 155 - protein, 6 aa, synthetic construct
GKMKIG

SEQ ID NO: 156 - protein, 6 aa, synthetic construct
GKCKDG

SEQ ID NO: 157 - protein, 7 aa, synthetic construct
GKRXRKG

SEQ ID NO: 158 - protein, 7 aa, synthetic construct
GKDXIKG

SEQ ID NO: 159 - protein, 7 aa, synthetic construct
GKRWRKG

SEQ ID NO: 160 - protein, 7 aa, synthetic construct
GKRARKG

SEQ ID NO: 161 - protein, 7 aa, synthetic construct
GKRMRKG

SEQ ID NO: 162 - protein, 7 aa, synthetic construct
GKRQRKG

SEQ ID NO: 163 - protein, 7 aa, synthetic construct
GKRTRKG

SEQ ID NO: 164 - protein, 7 aa, synthetic construct
GKRIRKG

SEQ ID NO: 165 - protein, 7 aa, synthetic construct
GKRERKG

SEQ ID NO: 166 - protein, 7 aa, synthetic construct
GKDVIKG

SEQ ID NO: 167 - protein, 7 aa, synthetic construct
GKRNRKG

SEQ ID NO: 168 - protein, 7 aa, synthetic construct
GKRHRKG

SEQ ID NO: 169 - protein, 7 aa, synthetic construct
GKDNIKG

SEQ ID NO: 170 - protein, 7 aa, synthetic construct
GKDFIKG

SEQ ID NO: 171 - protein, 7 aa, synthetic construct
GKDHIKG

SEQ ID NO: 172 - protein, 7 aa, synthetic construct
GKRRRKG

SEQ ID NO: 173 - protein, 7 aa, synthetic construct
GKDEIKG

SEQ ID NO: 174 - protein, 7 aa, synthetic construct
GKDPIKG

SEQ ID NO: 175 - protein, 7 aa, synthetic construct
GKDTIKG

SEQ ID NO: 176 - protein, 7 aa, synthetic construct
GKDSIKG

SEQ ID NO: 177 - protein, 7 aa, synthetic construct
GKDWIKG

SEQ ID NO: 178 - protein, 7 aa, synthetic construct
GKDDIKG

SEQ ID NO: 179 - protein, 7 aa, synthetic construct
GKRSRKG

SEQ ID NO: 180 - protein, 7 aa, synthetic construct
GKRFRKG

SEQ ID NO: 181 - protein, 7 aa, synthetic construct
GKRVRKG

SEQ ID NO: 182 - protein, 7 aa, synthetic construct
GKRDRKG

SEQ ID NO: 183 - protein, 7 aa, synthetic construct
GKDRIKG

SEQ ID NO: 184 - protein, 7 aa, synthetic construct
GKRLRKG

SEQ ID NO: 185 - protein, 7 aa, synthetic construct
GKRYRKG

SEQ ID NO: 186 - protein, 6 aa, synthetic construct
GKAIKG

SEQ ID NO: 187 - protein, 7 aa, synthetic construct
GKDCIKG

SEQ ID NO: 188 - protein, 7 aa, synthetic construct
GKDYIKG

SEQ ID NO: 189 - protein, 7 aa, synthetic construct
GKDGIKG

SEQ ID NO: 190 - protein, 14 aa, synthetic construct
KDDYAGGGAAGKDA

SEQ ID NO: 191 - protein, 6 aa, synthetic construct
AKGNAK

SEQ ID NO: 192 - protein, 6 aa, synthetic construct
GKAAKG

SEQ ID NO: 193 - protein, 6 aa, synthetic construct
GKGIKG

SEQ ID NO: 194 - protein, 6 aa, synthetic construct
GKIVKG

SEQ ID NO: 195 - protein, 6 aa, synthetic construct
GKLSKG

SEQ ID NO: 196 - protein, 6 aa, synthetic construct
GKQQKG

SEQ ID NO: 197 - protein, 6 aa, synthetic construct
GKATKG

SEQ ID NO: 198 - protein, 6 aa, synthetic construct
GKQVKG

SEQ ID NO: 199 - protein, 6 aa, synthetic construct
GKDIKG

SEQ ID NO: 200 - protein, 6 aa, synthetic construct
GKCQKG

SEQ ID NO: 201 - protein, 6 aa, synthetic construct
GKQGKG

SEQ ID NO: 202 - protein, 6 aa, synthetic construct
GKSSKG

SEQ ID NO: 203 - protein, 6 aa, synthetic construct
GKGTKG

SEQ ID NO: 204 - protein, 6 aa, synthetic construct
GKRVKG

SEQ ID NO: 205 - protein, 6 aa, synthetic construct
GKNPKG

SEQ ID NO: 206 - protein, 6 aa, synthetic construct
GKNLKG

SEQ ID NO: 207 - protein, 6 aa, synthetic construct
GKALKG

SEQ ID NO: 208 - protein, 6 aa, synthetic construct
GKQTKG

SEQ ID NO: 209 - protein, 6 aa, synthetic construct
GKGVKG

SEQ ID NO: 210 - protein, 6 aa, synthetic construct
GKHKPG

SEQ ID NO: 211 - protein, 6 aa, synthetic construct
GKLKDG

SEQ ID NO: 212 - protein, 6 aa, synthetic construct
GKIKQG

SEQ ID NO: 213 - protein, 6 aa, synthetic construct
GKGKPG

SEQ ID NO: 214 - protein, 6 aa, synthetic construct
GKVKSG

SEQ ID NO: 215 - protein, 6 aa, synthetic construct
GKHKVG

SEQ ID NO: 216 - protein, 6 aa, synthetic construct
GKHKDG

SEQ ID NO: 217 - protein, 6 aa, synthetic construct
GKDKEG

SEQ ID NO: 218 - protein, 6 aa, synthetic construct
GKGKAG

SEQ ID NO: 219 - protein, 6 aa, synthetic construct
GKMKLG

SEQ ID NO: 220 - protein, 6 aa, synthetic construct
GKIKCG

SEQ ID NO: 221 - protein, 6 aa, synthetic construct
GKPKDG

SEQ ID NO: 222 - protein, 6 aa, synthetic construct
GKPKEG

SEQ ID NO: 223 - protein, 6 aa, synthetic construct
GKLKAG

SEQ ID NO: 224 - protein, 6 aa, synthetic construct
GKGKDG

SEQ ID NO: 225 - protein, 6 aa, synthetic construct
GKEKMG

SEQ ID NO: 226 - protein, 6 aa, synthetic construct
GKQKDG

SEQ ID NO: 227 - protein, 6 aa, synthetic construct
GKMKNG

SEQ ID NO: 228 - protein, 6 aa, synthetic construct
GKGKCG

SEQ ID NO: 229 - protein, 6 aa, synthetic construct
GKTKPG

SEQ ID NO: 230 - protein, 6 aa, synthetic construct
GKDKNG

SEQ ID NO: 231 - protein, 6 aa, synthetic construct
GKCKGG

SEQ ID NO: 232 - protein, 6 aa, synthetic construct
GKSKNG

SEQ ID NO: 233 - protein, 6 aa, synthetic construct
GKLKGG

SEQ ID NO: 234 - protein, 6 aa, synthetic construct
GKPKPG

SEQ ID NO: 235 - protein, 6 aa, synthetic construct
GKLKQG

SEQ ID NO: 236 - protein, 6 aa, synthetic construct
GKSKCG

SEQ ID NO: 237 - protein, 6 aa, synthetic construct
GKSKMG

SEQ ID NO: 238 - protein, 6 aa, synthetic construct
GKGKVG

SEQ ID NO: 239 - protein, 6 aa, synthetic construct
GKTKIG

SEQ ID NO: 240 - protein, 6 aa, synthetic construct
GKIKEG

SEQ ID NO: 241 - protein, 6 aa, synthetic construct
GKHKRG

SEQ ID NO: 242 - protein, 6 aa, synthetic construct
GKIKMG

SEQ ID NO: 243 - protein, 6 aa, synthetic construct
GKSKGG

SEQ ID NO: 244 - protein, 6 aa, synthetic construct
GKVKRG

SEQ ID NO: 245 - protein, 6 aa, synthetic construct
GKSKPG

SEQ ID NO: 246 - protein, 6 aa, synthetic construct
GKLKRG

SEQ ID NO: 247 - protein, 6 aa, synthetic construct
GKSKRG

SEQ ID NO: 248 - protein, 7 aa, synthetic construct
GKDAIKG

SEQ ID NO: 249 - protein, 7 aa, synthetic construct
GKRGRKG

SEQ ID NO: 250 - protein, 7 aa, synthetic construct
GKRPRKG

SEQ ID NO: 251 - protein, 7 aa, synthetic construct
GKDQIKG

(19) United States
(12) Patent Application Publication
(10) Pub. No.: US 2025/0180575 A1
(43) Pub. Date: Jun. 5, 2025

(54) HIGH EFFICIENCY LABELS FOR BIOMOLECULAR ANALYSIS

(71) Applicant: BOARD OF REGENTS, THE UNIVERSITY OF TEXAS SYSTEM, Austtin, TX (US)

(72) Inventors: Edward MARCOTTE, Austin, TX (US); Eric V. ANSLYN, Austin, TX (US); Jagannath SWAMINATHAN, Austin, TX (US); Angela BARDO, Austin, TX (US); James BACHMAN, Los Angeles, CA (US); Caitlyn MCCAFFERTY, Austin, TX (US)

(21) Appl. No.: 18/725,234

(22) PCT Filed: Dec. 30, 2022
(86) PCT No.: PCT/US2022/082667

§ 371 Date: Jun. 28, 2024

Related U.S. Application Data
(60) Provisional application No. 63/295,339, filed on Dec. 30, 2021.

(51) Int. Cl. G01N 33/68; C07K 7/06; G01N 33/58

(52) U.S. Cl. CPC G01N 33/6809; C07K 7/06; G01N 33/582

(57) ABSTRACT

The present disclosure provides a detectable labeling reagent for coupling to a target biomolecule. The detectable labeling reagent may comprise a chemical handle configured to couple to the target biomolecule. The detectable labeling reagent may also comprise a backbone unit. The detectable labeling reagent may comprise one or more detectable moieties. The backbone unit may comprise a conformation so that, when the detectable labeling reagent is coupled to the biomolecule, the backbone unit can substantially constrain a position or an orientation of a detectable moiety of the one or more detectable moieties relative to another detectable moiety of the one or more detectable moieties that is coupled to the biomolecule.

### CROSS-REFERENCE

This application claims the benefit of U.S. Application No. 63/295,339, filed Dec. 30, 2021, which is incorporated by reference herein in its entirety.

### BACKGROUND

Biomolecule labeling is utilized in a wide range of diagnostic and descriptive assays. However, few labels are able to meet the requirements imposed by rapidly increasing assay sensitivities, multiplexing capabilities, and flexibilities. In particular, many labels develop signal aberrations through inter-label interactions, and therefore are unsuitable for close packing at the molecular level. For example, many fluorescent dyes quench over multi-nanometer distances, rendering many single molecule labeling experiments infeasible.

Signal aberrations from inter-label interactions can be problematic for a wide range of assays. While microscopy methodologies have continually improved over recent decades, now often circumventing diffraction limitations to enable nanometer-scale resolution, assays are nonetheless often limited to tens or hundreds of nanometer resolutions due to chromatic aberrations stemming from inter-dye interactions. Furthermore, many forms of biopolymer (e.g., nucleic acid, polyketide, and peptide) analysis require proximal monomer labeling for which most modern labels are unsuitable. In particular, peptide fluorosequencing systems often comprise peptides with pluralities of consecutive labeled amino acids, and thus require labels whose aggregate signal intensity is not affected by local copy number.

### SUMMARY

The present disclosure provides labeling reagents with diminished signal aberrations (e.g., fluorophore quenching and chromatic shifts) resulting from inter-label interactions. The labels of the present disclosure may provide quantitative signal intensities in single molecule multiple labeling assays. The labels may also chemical and sub-molecular coupling specificities, thereby enabling sensitive biomolecular discrimination.

In an aspect, the present disclosure provides a detectable labeling reagent for coupling to a target biomolecule, the detectable labeling reagent comprising: (i) a chemical handle configured to couple to the target biomolecule, (ii) a backbone unit, and (iii) one or more detectable moieties; wherein the backbone unit comprises a conformation so that, when the detectable labeling reagent is coupled to the target biomolecule, the backbone unit substantially constrains a position or an orientation of a detectable moiety of the one or more detectable moieties relative to another detectable moiety of the one or more detectable moieties that is coupled to the target biomolecule.

In some embodiments of any of the detectable labeling reagents of the present disclosure, the chemical handle is configured to couple to an amino acid of the target biomolecule.

In some embodiments of any of the detectable labeling reagents of the present disclosure, the detectable labeling reagent comprises two detectable moieties.

In some embodiments of any of the detectable labeling reagents of the present disclosure, the chemical handle comprises a primary amine-reactive group, a primary thiol-reactive group, a thioether-reactive group, a primary alcohol-reactive group, a phenol-reactive group, a carboxylic acid-reactive group, or a hydroxyl-reactive group, or any combination thereof.

In some embodiments of any of the detectable labeling reagents of the present disclosure, the detectable labeling reagent further comprises a structure according to:

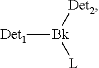

wherein Bk denotes the backbone unit; Det₁ and Det₂ independently denote a first and a second detectable moiety; and L denotes the chemical handle.

In some embodiments of any of the detectable labeling reagents of the present disclosure, the backbone unit further comprises an oligopeptide structure according to: X₃-X₁-X₂-X₄ wherein X₃ and X₄ independently denote an amino acid side chain linked to a detectable moiety; and X₁ and X₂ independently denote any amino acid.

In some embodiments of any of the detectable labeling reagents of the present disclosure, the backbone unit further comprises an oligopeptide structure according to: X₃-X₁-X₂-X₄ wherein;
X₃ comprises

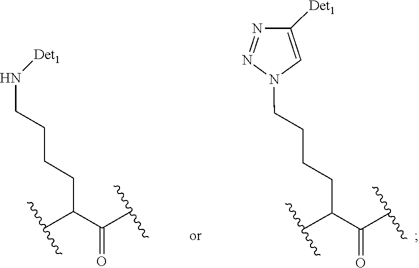

X₄ comprises

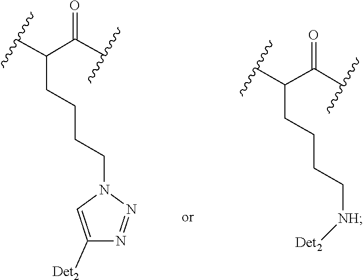

Det₁ and Det₂ independently denote first and second detectable moieties; and X₁ and X₂ independently denote any amino acid. In some embodiments, the detectable label further comprises an oligopeptide structure according to: Ac-G-X₃-X₁-X₂-X₄-L; wherein Ac denotes an acetyl group; G denotes glycine; and L denotes the chemical handle.

In some embodiments of any of the detectable labeling reagents of the present disclosure, the backbone unit substantially constrains an orientation of the first and the second detectable moiety relative to each other. In some embodiments, the orientation of the first and the second detectable moieties comprises an average angular deviation of at most about 160 degrees relative to each other. In some embodiments, the orientation of the first and the second detectable moieties comprises an average angular deviation of at least 60 degrees relative to each other. In some embodiments, the backbone unit comprises an oligopeptide according to: X₃-X₁-X₂-X₄, wherein: X₁ is selected from the group consisting of A, Q, R, M, H, L, D, N, E, I, and C; and X₂ is selected from the group consisting of Q, P, E, L, R, V, T, M, I, H, and C. In some embodiments, the backbone unit comprises an oligopeptide according to: X₃-X₁-X₂-X₄, wherein: X₁-X₂ is according to: AQ, QP, RE, MP, HL, RR, QV, LT, DT, NT, EM, NI, DI, RH, RT, NC, IP, AE, CE, RM, EP, or HT.

In some embodiments of any of the detectable labeling reagents of the present disclosure, the backbone unit substantially constrains a distance of the first and the second detectable moiety relative to each other. In some embodiments, the distance of the one or more detectable moieties comprises a distance of at most about 25 angstroms. In some embodiments, the distance of the one or more detectable moieties comprises at least about 5 angstroms.

In some embodiments of any of the detectable labeling reagents of the present disclosure, the backbone unit comprises an oligopeptide according to: G-K-X₁-X₂-K-G, wherein: X₁ is selected from the group consisting of A, E, N, C, P, D, S, R, M, L, G, N, and H; and X₂ is selected from the group consisting of S, P, I, E, L, V, P, H, T, V, Q, and R.

In some embodiments of any of the detectable labeling reagents of the present disclosure, the backbone unit comprises X₁-X₂ according to: AS, AP, EP, NI, CE, PS, DL, SV, RP, RH, DT, MV, DQ, LS, GV, NQ DI, HT, or RR.

In some embodiments of any of the detectable labeling reagents of the present disclosure, the backbone unit substantially constrains an average deviation of the one or more detectable moieties comprises relative to the chemical handle. In some embodiments, the average deviation comprises at most about 5 angstroms. In some embodiments, the average deviation comprises at least about 1 angstrom. In some embodiments, the backbone unit comprises an oligopeptide according to: X₃-X₁-X₂-X₄, wherein: X₁ is selected from the group consisting of G, N, I, L, Q, A, D, C, S, and R; and X₂ is selected from the group consisting of I, Q, G, V, S, T, P, and L.

In some embodiments, the backbone unit comprises an oligopeptide according to: X₃-X₁-X₂-X₄, wherein: X₁-X₂ is according to: GI, NQ, GG, IV, LS, QQ, AT, QV, DI, CQ, QG, SS, GT, RV, NP, NL, AL, QT, or GV.

In some embodiments of any of the detectable labeling reagents of the present disclosure, the conformation of the backbone unit substantially constrains an orientation of the one or more detectable moieties relative to the chemical handle.

In some embodiments of any of the detectable labeling reagents of the present disclosure, the orientation of the one or more detectable moieties comprises an average angular deviation of at most about 60 degrees relative to the chemical handle.

In some embodiments of any of the detectable labeling reagents of the present disclosure, the orientation of the one or more detectable moieties comprises an average angular deviation of at most about 45 degrees relative to the chemical handle.

In some embodiments of any of the detectable labeling reagents of the present disclosure, the orientation of the one or more detectable moieties comprises an average angular deviation of at most about 30 degrees relative to the chemical handle.

In some embodiments of any of the detectable labeling reagents of the present disclosure, the conformation of the backbone unit substantially constrains a position of the one or more detectable moieties relative to the chemical handle.

In some embodiments of any of the detectable labeling reagents of the present disclosure, the position of the one or more detectable moieties comprises an average deviation of at most about 10 nanometers (nm) relative to the chemical handle.

In some embodiments of any of the detectable labeling reagents of the present disclosure, the position of the one or more detectable moieties comprises an average deviation of at most about 6 nanometer (nm) relative to the chemical handle.

In some embodiments of any of the detectable labeling reagents of the present disclosure, the position of the one or more detectable moieties comprises an average deviation of at most about 4 nanometers (nm) relative to the chemical handle.

In some embodiments of any of the detectable labeling reagents of the present disclosure, the position or the orientation of the one or more detectable moieties is an average position relative to the chemical handle.

In some embodiments of any of the detectable labeling reagents of the present disclosure, the position of the one or more detectable moieties is at least 1 nanometer (nm) from the chemical handle.

In some embodiments of any of the detectable labeling reagents of the present disclosure, the position of the one or more detectable moieties is at least 2 nanometers (nm) from the chemical handle.

In some embodiments of any of the detectable labeling reagents of the present disclosure, the position of the one or more detectable moieties is at least 5 nanometers (nm) from the chemical handle.

In some embodiments of any of the detectable labeling reagents of the present disclosure, the backbone unit comprises the conformation when coupled to the target biomolecule.

In some embodiments of any of the detectable labeling reagents of the present disclosure, the conformation comprises stability at 60° C.

In some embodiments of any of the detectable labeling reagents of the present disclosure, the backbone unit comprises a peptide. In some embodiments, the peptide comprises between 4 and 20 amino acids. In some embodiments, the peptide comprises between 6 and 15 amino acids. In some embodiments, the peptide comprises between 8 and 12 amino acids. In some embodiments, the peptide comprises at least 3 unique amino acids. In some embodiments, the peptide comprises at least 5 unique amino acids. In some embodiments, the peptide comprises a non-natural amino acid.

In some embodiments, the peptide comprises a non-amino acid moiety. In some embodiments, the non-amino acid moiety is coupled to at least two amino acids of the oligopeptide backbone unit. In some embodiments, the conformation comprises a secondary structural feature of the peptide. In some embodiments, the secondary structural feature comprises an alpha-helix. In some embodiments, the peptide comprises a disulfide bond.

In some embodiments of any of the detectable labeling reagents of the present disclosure, the backbone unit comprises a second conformation, and wherein the backbone unit is configured to interconvert between the conformation and the second conformation. In some embodiments, an intensity of a signal of the one or more detectable moieties is greater when the backbone unit comprises the conformation than when the backbone unit comprises the second conformation. In some embodiments, a wavelength of a signal of the one or more detectable moieties changes when the backbone unit converts between the conformation and the second conformation. In some embodiments, a wavelength of a signal of the one or more detectable moieties is greater when the backbone unit comprises the conformation than when the backbone unit comprises the second conformation. In some embodiments, interconversion of the backbone unit between the conformation and the second conformation is light mediated. In some embodiments, interconversion of the backbone unit between the conformation and the second conformation is temperature mediated. In some embodiments, interconversion of the backbone unit between the conformation and the second conformation is chemically mediated. In some embodiments, interconversion of the backbone unit between the conformation and the second conformation is pH mediated.

In some embodiments of any of the detectable labeling reagents of the present disclosure, the backbone unit is linear.

In some embodiments of any of the detectable labeling reagents of the present disclosure, the backbone unit is branched.

In some embodiments of any of the detectable labeling reagents of the present disclosure, the backbone unit comprises a cyclic or polycyclic structure.

In some embodiments of any of the detectable labeling reagents of the present disclosure, the chemical handle is inert towards the backbone unit and the one or more detectable moieties.

In some embodiments of any of the detectable labeling reagents of the present disclosure, the chemical handle is configured to selectively couple to an amino acid type. In some embodiments, the amino acid type comprises cysteine, lysine, tyrosine, histidine, aspartic acid, glutamic acid, tryptophan, arginine, methionine, or any combination thereof. In some embodiments, the amino acid type comprises cysteine, and wherein the chemical handle comprises an iodoacetamide, a thiol, a benzyl halide, an allyl halide, a selenocyanate, a maleimide, an alkyne, or any combination thereof.

In some embodiments, the amino acid type comprises lysine, and wherein the chemical handle comprises a thiocyanate, an isothiocyanate, a maleimide, an aldehyde, an isatoic anhydride, an NHS ester, or any combination thereof. In some embodiments, the amino acid type comprises aspartic acid or glutamic acid, and wherein the chemical handle comprises an amine, an alcohol, a thiol, an organocuprate, or any combination thereof. In some embodiments, the amino acid type comprises tyrosine, and wherein the chemical handle comprises a diazonium compound. In some embodiments, the amino acid type comprises histidine, and wherein the chemical handle comprises an alpha-beta unsaturated carbonyl compound, an epoxide, or a combination thereof. In some embodiments, the amino acid type comprises arginine, and wherein the chemical handle comprises an NHS ester. In some embodiments, the amino acid type comprises methionine, and wherein the chemical handle comprises an oxaziridine compound. In some embodiments, the amino acid type comprises tryptophan, and wherein the chemical handle comprises a diazopropanoate ester.

In some embodiments, the amino acid type comprises a post-translationally modified amino acid type. In some embodiments, the post-translationally modified amino acid type comprises phosphorylation, glycosylation, nitrosylation, citrullination, sulfenylation, trimethylation, or any combination thereof. In some embodiments, the post-translationally modified amino acid type comprises phosphoserine, phosphotyrosine, phosphothreonine, or any combination thereof, and wherein the chemical handle comprises a disulfide. In some embodiments, the amino acid type comprises an N-terminal amino acid or a C-terminal amino acid.

In some embodiments of any of the detectable labeling reagents of the present disclosure, the chemical handle is configured to couple to form a single attachment to the biomolecule.

In some embodiments of any of the detectable labeling reagents of the present disclosure, the chemical handle is configured to stoichiometrically couple to the biomolecule.

In some embodiments of any of the detectable labeling reagents of the present disclosure, further comprising a triplet state quencher.

In some embodiments of any of the detectable labeling reagents of the present disclosure, the one or more detectable moieties of the detectable labeling reagent comprises a half-life which is at least twice as long as a half-life of the one or more detectable moieties provided without the labeling reagent.

In some embodiments of any of the detectable labeling reagents of the present disclosure, the backbone unit increases a half-life of the one or more detectable moieties by at least 50%.

In an aspect, the present disclosure provides a system comprising a biopolymer comprising a plurality of subunits, wherein a first subunit of the plurality of subunits is coupled to a first detectable label, wherein a second subunit of the plurality of subunits is coupled to a second detectable label, wherein the first and the second detectable labels each comprise: (i) a chemical handle for the coupling to the first and the second subunits, (ii) a backbone unit, and (iii) one or more detectable moieties; wherein the chemical handle of the first label is no more than 25 Å from the chemical handle of the second label, and wherein a combined intensity of a detectable signal of the first detectable label and the second detectable label is greater than an intensity of either the first or the second detectable label taken alone.

In some embodiments, the first or the second detectable labels independently comprise a detectable labeling reagent according to any of the detectable labeling regents of the present disclosure coupled to the first subunit or the second subunit.

In an aspect, the present disclosure provides a detectable labeling reagent for coupling to a biomolecule, the detectable labeling reagent comprising: i) a backbone unit, ii) a first fluorophore, iii) a second fluorophore, and iv) a functional handle for coupling to the biomolecule; wherein the first fluorophore and the second fluorophore are separated by at most 30 Å, and wherein a fluorescence intensity of the detectable labeling reagent is greater than a fluorescence intensity of the first fluorophore in the absence of the second fluorophore and a fluorescence intensity of the second fluorophore in the absence of the first fluorophore.

In some embodiments, the detectable labeling reagent comprises a structure according to any of the detectable labeling regents of the present disclosure.

In some embodiments of any of the detectable labeling reagents of the present disclosure, the backbone unit comprises an oligopeptide with about 4 to about 20 amino acids.

In some embodiments of any of the detectable labeling reagents of the present disclosure, the backbone unit comprises between about 6 and about 15 amino acids. In some embodiments, the backbone unit comprises between about 8 and about 12 amino acids.

In some embodiments of any of the detectable labeling reagents of the present disclosure, the backbone unit comprises at least 4 types of amino acids. In some embodiments, the oligopeptide backbone unit comprises at least 6 types of amino acids.

In some embodiments of any of the detectable labeling reagents of the present disclosure, the oligopeptide backbone unit comprises a secondary structural feature. In some embodiments, the secondary structural feature is stable at 60° C. In some embodiments, the secondary structural feature comprises an alpha-helix. In some embodiments, the oligopeptide backbone unit prevents contact between: (1) the functional handle, and (2) the first and the second fluorophores. In some embodiments, the oligopeptide backbone unit comprises a disulfide bond. In some embodiments, the oligopeptide backbone unit is configured to interconvert between a first state and a second state, and wherein a fluorescence intensity of the first fluorophore or the second fluorophore differs between the first state and the second state of the oligopeptide backbone unit. In some embodiments, the oligopeptide backbone unit comprises different conformations in the first state and the second state. In some embodiments, the interconverting between the first state and the second state comprises chemical interconversion. In some embodiments, the interconverting between the first state and the second state is temperature mediated, chemical condition mediated, or light mediated. In some embodiments, the oligopeptide backbone unit is linear. In some embodiments, the backbone unit is branched. In some embodiments, the backbone unit comprises a non-natural amino acid. In some embodiments, the backbone unit comprises a non-amino acid moiety. In some embodiments, the non-amino acid moiety is coupled to at least two amino acids of the backbone unit. In some embodiments, an absorbance maximum of the first fluorophore and an absorbance maximum of the second fluorophore are within 10 nm. In some embodiments, an emission maximum of the first fluorophore and an emission maximum of the second fluorophore are within 10 nm. In some embodiments, the first fluorophore is identical to the second fluorophore. In some embodiments, the first fluorophore and the second fluorophore are separated by at most 20 Å. In some embodiments, the first fluorophore and the second fluorophore are separated by at most 12 Å.

In some embodiments, a relative orientation of the first fluorophore and the second fluorophore is constrained by the backbone. In some embodiments, a dipole moment of the first fluorophore is substantially aligned with a dipole moment of the second fluorophore. In some embodiments, the first fluorophore is constrained from rotating relative to the second fluorophore.

In some embodiments of any of the detectable labeling reagents of the present disclosure, the functional handle is inert towards the oligopeptide unit, the first fluorophore, and the second fluorophore.

In some embodiments of any of the detectable labeling reagents of the present disclosure, the functional handle is configured to selectively couple to an amino acid type. In some embodiments, the amino acid type comprises cysteine, lysine, tyrosine, histidine, aspartic acid, glutamic acid, tryptophan, arginine, methionine, or any combination thereof. In some embodiments, the amino acid type comprises cysteine, and wherein the chemical moiety comprises an iodoacetamide, a thiol, a benzyl halide, an allyl halide, a selenocyanate, a maleimide, an alkyne, or any combination thereof. In some embodiments, the amino acid type comprises lysine, and wherein the chemical moiety comprises a thiocyanate, an isothiocyanate, a maleimide, an aldehyde, an isatoic anhydride, an NHS ester, or any combination thereof. In some embodiments, the amino acid type comprises aspartic acid or glutamic acid, and wherein the chemical moiety comprises an amine, an alcohol, a thiol, an organocuprate, or any combination thereof. In some embodiments, the amino acid type comprises tyrosine, and wherein the chemical moiety comprises a diazonium compound. In some embodiments, the amino acid type comprises histidine, and wherein the chemical moiety comprises an alpha-beta unsaturated carbonyl compound, an epoxide, or a combination thereof. In some embodiments, the amino acid type comprises arginine, and wherein the chemical moiety comprises an NHS ester. In some embodiments, the amino acid type comprises methionine, and wherein the chemical moiety comprises an oxaziridine compound. In some embodiments, the amino acid type comprises tryptophan, and wherein the chemical moiety comprises a diazopropanoate ester. In some embodiments, the amino acid type comprises a post-translationally modified amino acid type. In some embodiments, the post-translationally modified amino acid type comprises phosphorylation, glycosylation, nitrosylation, citrullination, sulfenylation, trimethylation, or any combination thereof. In some embodiments, the post-translationally modified amino acid type comprises phosphoserine, phosphotyrosine, phosphothreonine, or any combination thereof, and wherein the chemical moiety comprises a disulfide. In some embodiments, the amino acid type comprises an N-terminal amino acid or a C-terminal amino acid.

In some embodiments of any of the detectable labeling reagents of the present disclosure, the functional handle is configured to couple to form a single attachment to the biomolecule.

In some embodiments of any of the detectable labeling reagents of the present disclosure, the functional handle is configured to stoichiometrically couple to the biomolecule.

In some embodiments of any of the detectable labeling reagents of the present disclosure, further comprising a triplet state quencher.

In some embodiments of any of the detectable labeling reagents of the present disclosure, the first fluorophore or the second fluorophore comprises a plurality of fluorophores.

In some embodiments of any of the detectable labeling reagents of the present disclosure, the first fluorophore and the second fluorophore each comprise a plurality of fluorophores.

In some embodiments of any of the detectable labeling reagents of the present disclosure, the detectable labeling reagent is inert towards Edman degradation.

In some embodiments of any of the detectable labeling reagents of the present disclosure, the detectable labeling reagent comprises an aqueous solubility of at least 2 mg/mL. In some embodiments, the detectable labeling reagent comprises an aqueous solubility of at least 20 mg/mL.

In some embodiments of any of the detectable labeling reagents of the present disclosure, the first fluorophore comprises a diminished rate of photobleaching than the first fluorophore in the absence of the backbone unit, the second fluorophore, and the functional handle.

In an aspect, the present disclosure provides a system comprising a peptide, wherein the peptide comprises an amino acid type, and wherein each of a plurality of amino acids of the amino acid type are coupled any of the detectable labeling reagent of the present disclosure.

In some embodiments, the peptide is immobilized to a substrate. In some embodiments, the substrate comprises a bead, a polymer matrix, a surface, or a slide.

In some embodiments, a C-terminus of the peptide is coupled to the substrate.

In some embodiments, further comprising a dye repellant. In some embodiments, a distance between the first fluorophore and the second fluorophore is greater than the distance in the absence of the dye-repellant. In some embodiments, the dye repellant comprises a counterion for the first fluorophore or the second fluorophore. In some embodiments, the dye repellant comprises an anion.

In some embodiments, the dye repellant comprises a dicarboxylic acid compound. In some embodiments, the dicarboxylic acid compound comprises a benzenedicarboxylic acid.

In some embodiments, a conformation of the peptide is substantially similar to a conformation of the peptide in the absence of the optically detectable labeling reagent.

In some embodiments, a solubility of the peptide is similar to or greater than a solubility of the peptide in an absence of the optically detectable labeling reagent.

In some embodiments, an intensity of a fluorescence signal of the detectable labeling reagent is substantially linear with respect to the number of the plurality of amino acids coupled to the optically detectable labeling reagent.

In some embodiments, two amino acids of the plurality of amino acids coupled to the detectable labeling reagent are adjacent with respect to a sequence of the peptide.

In some embodiments, an intensity of a fluorescence signal of the detectable labeling reagent coupled to the two amino acids is approximately twice that of the detectable labeling reagent in a dilute solution.

In some embodiments, the peptide comprises a second amino acid type, and wherein a plurality of amino acids of the second amino acid type are coupled to any of the second detectable labeling reagent of the present disclosure. In some embodiments, the first fluorophore and the second fluorophore of the detectable labeling reagent are different than the first fluorophore and the second fluorophore of the second optically detectable labeling reagent. In some embodiments, the detectable labeling reagent and the second detectable labeling reagent generate different fluorescence signals.

In an aspect, the present disclosure provides a method for analyzing a peptide, the method comprising: (a) coupling a detectable labeling reagent to an amino acid of the peptide, wherein the labeling reagent comprises a backbone unit, a plurality of fluorophores, and a functional handle for the coupling to the amino acid of the peptide, (b) detecting a signal from the labeling reagent coupled to the peptide, and (c) using the signal to identify the amino acid of the peptide.

In some embodiments, the detectable labeling reagent comprises any of the detectable labeling reagents of the present disclosure.

In some embodiments, the functional handle comprises specificity for the amino acid of the peptide.

In some embodiments, the amino acid comprises cysteine, lysine, tyrosine, histidine, aspartic acid, glutamic acid, tryptophan, arginine, methionine, N-terminal amino acids, C-terminal amino acids, or any combination thereof. In some embodiments, the amino acid comprises N-terminal amino acids or C-terminal amino acids. In some embodiments, the functional handle comprises pyridinecarboxyaldehyde (PCA).

In some embodiments, the labeling reagent couples to the peptide with about 1:1 stoichiometry.

In some embodiments, (c) further comprises quantifying a concentration or abundance of the peptide. In some embodiments, the concentration of the peptide is less than about 1 pM.

In some embodiments, the amino acid comprises a plurality of amino acids, and wherein the labeling reagent couples to at least a subset of the plurality of amino acids. In some embodiments, the labeling reagent couples to each of the plurality of amino acids. In some embodiments, (c) comprises quantifying the plurality of amino acids of the peptide.

In some embodiments, further comprising (d) cleaving at least a portion of the peptide, and (e) detecting a signal or a signal change from the labeling reagent coupled to the peptide. In some embodiments, further comprising (f) identifying a sequence of the peptide based at least in part on the signal or the signal change in (e).

In some embodiments, the detecting comprises imaging the labeling reagent coupled to the peptide.

In an aspect, the present disclosure provides a detectable labeling reagent (e.g., optically detectable labeling reagent) for coupling to a target biomolecule, the optically detectable labeling reagent comprising: (i) a chemical handle configured to couple to the target biomolecule, (ii) a backbone unit, and (iii) one or more detectable moieties; wherein the backbone unit comprises a conformation so that, when the detectable labeling reagent is coupled to the target biomolecule, the backbone unit substantially constrains a position or an orientation of a detectable moiety of the one or more detectable moieties relative to another detectable moiety of the one or more detectable moieties that is coupled to the target biomolecule. In some embodiments, the conformation may be a stable conformation.

In some embodiments, the conformation of the backbone unit substantially constrains an orientation of the one or more detectable moieties relative to the chemical handle. In some embodiments, the one or more detectable moieties comprises two or more detectable moieties. In some embodiments, the orientation of the one or more detectable moieties comprises an average angular deviation of at most about 60 degrees relative to the chemical handle. In some embodiments, the orientation of the one or more detectable moieties comprises an average angular deviation of at most about 45 degrees relative to the chemical handle. In some embodiments, the orientation of the one or more detectable moieties comprises an average angular deviation of at most about 30 degrees relative to the chemical handle. In some embodiments, the conformation of the backbone unit substantially constrains a position of the one or more detectable moieties relative to the chemical handle.

In some embodiments, the angle or orientation of the one or more detectable moieties is substantially constrained relative to another detectable moiety. In some embodiments, the backbone unit substantially constrains an angle or orientation of the first and the second detectable moiety relative to each other. In some cases, the orientation or angle is at most about 160 degrees, at most about 150 degrees, at most about 140 degrees, at most about 120 degrees, at most about 110 degrees, at most about 100 degrees, at most about 90 degrees, at most about 80 degrees, or at most about 70 degrees. In some cases, the orientation or angle is at least about 60 degrees, at least about 70 degrees, at least about 80 degrees, at least about 90 degrees, at least about 100 degrees, at least about 110 degrees, at least about 120 degrees, at least about 130 degrees, at least about 140 degrees, or at least about 150 degrees.

In some embodiments, the distance of the one or more detectable moieties is substantially constrained relative to another detectable moiety. In some embodiments, the backbone unit substantially constrains a distance of the first and the second detectable moiety relative to each other. In some embodiments, the distance between the first and the second detectable moiety is between about 5 Å to about 25 Å. In some embodiments, the distance between the first and the second detectable moiety is at least about 5 Å, at least about 6 Å, at least about 7 Å, at least about 8 Å, at least about 9 Å, at least about 10 Å, at least about 11 Å, at least about 12 Å, at least about 14 Å, at least about 15 Å, at least about 16 Å, at least about 17 Å, at least about 18 Å, at least about 19 Å, at least about 20 Å, at least about 21 Å, at least about 22 Å, at least about 23 Å, at least about 24 Å, at least about 25 Å, or more. In some embodiments, the distance between the first and the second detectable moiety is at most about 25 Å, at most about 24 Å, at most about 23 Å, at most about 22 Å, at most about 21 Å, at most about 20 Å, at most about 19 Å, at most about 18 Å, at most about 17 Å, at most about 16 Å, at most about 15 Å, at most about 14 Å, at most about 13 Å, at most about 12 Å, at most about 11 Å, at most about 10 Å, at most about 9 Å, at most about 8 Å, at most about 7 Å, at most about 6 Å, at most about 5 Å, or less. In some embodiments, the distance between the first and second detectable moiety is about 5 Å, about 6 Å, about 7 Å, about 8 Å, about 9 Å, about 10 Å, about 11 Å, about 12 Å, about 13 Å, about 14 Å, about 15 Å, about 16 Å, about 17 Å, about 18 Å, about 19 Å, about 20 Å, about 21 Å, about 22 Å, about 23 Å, about 24 Å, or about 25 Å.

In some embodiments, the position of the one or more detectable moieties comprises an average deviation of at most about 10 angstroms relative to the chemical handle. In some embodiments, the position of the one or more detectable moieties comprises an average deviation of at most about 6 angstroms relative to the chemical handle. In some embodiments, the position of the one or more detectable moieties comprises an average deviation of at most about 4 angstroms relative to the chemical handle. In some embodiments, the position or the orientation of the one or more detectable moieties is an average position relative to the chemical handle. In some embodiments, the position of the one or more detectable moieties is at least 1 angstrom from the chemical handle. In some embodiments, the position of the one or more detectable moieties is at least 2 angstroms from the chemical handle. In some embodiments, the position of the one or more detectable moieties is at least 5 angstroms from the chemical handle. In some embodiments, the position of the one or more detectable moieties comprises an average deviation of at most about 10 nanometers (nm) relative to the chemical handle. In some embodiments, the position of the one or more detectable moieties comprises an average deviation of at most about 6 nanometer (nm) relative to the chemical handle. In some embodiments, the position of the one or more detectable moieties comprises an average deviation of at most about 4 nanometers (nm) relative to the chemical handle. In some embodiments, the position or the orientation of the one or more detectable moieties is an average position relative to the chemical handle. In some embodiments, the position of the one or more detectable moieties is at least 1 nanometer (nm) from the chemical handle. In some embodiments, the position of the one or more detectable moieties is at least 2 nanometers (nm) from the chemical handle. In some embodiments, the position of the one or more detectable moieties is at least 5 nanometers (nm) from the chemical handle. In some embodiments, the backbone unit comprises the conformation when coupled to the target biomolecule. In some embodiments, the conformation comprises stability at 60° C. In some embodiments, the backbone unit comprises a peptide. In some embodiments, the peptide comprises between 4 and 20 amino acids. In some embodiments, the peptide comprises between 6 and 15 amino acids. In some embodiments, the peptide comprises between 8 and 12 amino acids. In some embodiments, the peptide comprises at least 3 unique of amino acids. In some embodiments, the peptide comprises at least 5 unique of amino acids. In some embodiments, the peptide comprises a non-natural amino acid. In some embodiments, the peptide comprises a non-amino acid moiety. In some embodiments, the non-amino acid moiety is coupled to at least two amino acids of the oligopeptide backbone unit. In some embodiments, the conformation comprises a secondary structural feature of the peptide. In some embodiments, the secondary structural feature comprises an alpha-helix. In some embodiments, the peptide comprises a disulfide bond.

In some embodiments, the backbone unit comprises a second conformation, and wherein the backbone unit is configured to interconvert between the conformation and the second conformation. In some embodiments, an intensity of a signal of the one or more detectable moieties is greater when the backbone unit comprises the conformation than when the backbone unit comprises the second conformation. In some embodiments, a wavelength of a signal of the one or more detectable moieties changes when the backbone unit converts between the conformation and the second conformation. In some embodiments, a wavelength of a signal of the one or more detectable moieties is greater when the backbone unit comprises the conformation than when the backbone unit comprises the second conformation. In some embodiments, interconversion of the backbone unit between the conformation and the second conformation is light mediated. In some embodiments, interconversion of the backbone unit between the conformation and the second conformation is temperature mediated. In some embodiments, interconversion of the backbone unit between the conformation and the second conformation is chemically mediated. In some embodiments, interconversion of the backbone unit between the conformation and the second conformation is pH mediated.

In some embodiments, the backbone unit is linear. In some embodiments, the backbone unit is branched. In some embodiments, the backbone unit comprises a cyclic or polycyclic structure. In some embodiments, the chemical handle is inert towards the backbone unit and the one or more detectable moieties. In some cases, the chemical handle comprises an amino acid side-chain reactive moiety. In some embodiments, the chemical handle comprises a primary amine-reactive group, a sulhydryl-reactive group, a thioether-reactive group, a primary alcohol-reactive group, a phenol-reactive group, a carboxylic acid-reactive group, or a hydroxyl-reactive group. In some cases, the chemical handle comprises an electrophile. In some embodiments, the chemical handle comprises a thiol, a primary thiol-reactive group (e.g. a haloacetamide, an allyl halide, a selenocyanate, a maleimide, an alkyne, a tosylate, an azide, or any combination thereof), or a secondary thiol-reactive electrophile (e.g. a benzyl halide). In some embodiments, the chemical handle comprises a primary amine-reactive electrophile (e.g. a thiocyanate, an isothiocyanate, a maleimide, an aldehyde, an isatoic anhydride, an NHS ester, or any combination thereof). In some embodiments, the chemical handle is configured to selectively couple to an amino acid type. In some embodiments, the amino acid type comprises cysteine, lysine, tyrosine, histidine, aspartic acid, glutamic acid, tryptophan, arginine, methionine, or any combination thereof. In some embodiments, the amino acid type comprises cysteine, and wherein the chemical handle comprises an iodoacetamide, a thiol, a benzyl halide, an allyl halide, a selenocyanate, a maleimide, an alkyne, a tosylate, an azide, or any combination thereof. In some embodiments, the amino acid type comprises lysine, and wherein the chemical handle comprises a thiocyanate, an isothiocyanate, a maleimide, an aldehyde, an isatoic anhydride, an NHS ester, or any combination thereof. In some embodiments, the amino acid type comprises aspartic acid or glutamic acid, and wherein the chemical handle comprises an amine, an alcohol, a thiol, an organocuprate, or any combination thereof. In some embodiments, the amino acid type comprises tyrosine, and wherein the chemical handle comprises a diazonium compound. In some embodiments, the amino acid type comprises histidine, and wherein the chemical handle comprises an alpha-beta unsaturated carbonyl compound, an epoxide, or a combination thereof. In some embodiments, the amino acid type comprises arginine, and wherein the chemical handle comprises an NHS ester. In some embodiments, the amino acid type comprises methionine, and wherein the chemical handle comprises an oxaziridine compound. In some embodiments, the amino acid type comprises tryptophan, and wherein the chemical handle comprises a diazopropanoate ester. In some embodiments, the amino acid type comprises a post-translationally modified amino acid type. In some embodiments, the post-translationally modified amino acid type comprises phosphorylation, glycosylation, nitrosylation, citrullination, sulfenylation, trimethylation, or any combination thereof. In some embodiments, the post-translationally modified amino acid type comprises phosphoserine, phosphotyrosine, phosphothreonine, or any combination thereof, and wherein the chemical handle comprises a disulfide. In some embodiments, the amino acid type comprises an N-terminal amino acid or a C-terminal amino acid. In some embodiments, the chemical handle is configured to couple to form a single attachment to the biomolecule.

In some embodiments, the chemical handle is configured to stoichiometrically couple to the biomolecule. In some embodiments, the detectable labeling reagent further comprising a triplet state quencher. In some embodiments, the one or more detectable moieties of the detectable labeling reagent comprises a half-life which is at least twice as long as a half-life of the one or more detectable moieties provided without the labeling reagent. In some embodiments, the backbone unit increases a half-life of the one or more detectable moieties by at least 50%.

In another aspect, the present disclosure provides a system comprising a biopolymer comprising a plurality of subunits, wherein a first subunit of the plurality of subunits is coupled to a first detectable label, wherein a second subunit of the plurality of subunits is coupled to a second detectable label, wherein the first and the second detectable labels each comprise: (i) a chemical handle for the coupling to the first and the second subunits, (ii) a backbone unit, and (iii) one or more detectable moieties; wherein the chemical handle of the first label is no more than 25 Å from the chemical handle of the second label, and wherein a combined intensity of a detectable signal of the first detectable label and the second detectable label is greater than an intensity of either the first or the second detectable label taken alone. In some embodiments, the one or more detectable moieties comprises two or more detectable moieties.

In another aspect, the present disclosure provides a detectable labeling reagent (e.g., an optically detectable labeling reagent) for coupling to a biomolecule, the optically detectable labeling reagent comprising: (i) a backbone unit, (ii) a first fluorophore, (iii) a second fluorophore, and (iv) a functional handle for coupling to the biomolecule; wherein the first fluorophore and the second fluorophore are separated by at most 30 Å, and wherein a fluorescence intensity of the optically detectable labeling reagent is greater than a fluorescence intensity of the first fluorophore in the absence of the second fluorophore and a fluorescence intensity of the second fluorophore in the absence of the first fluorophore.

In some embodiments, the backbone unit comprises an oligopeptide with about 4 to about 20 amino acids. In some embodiments, the backbone unit comprises between about 6 and about 15 amino acids. In some embodiments, the backbone unit comprises between about 8 and about 12 amino acids. In some embodiments, the backbone unit comprises at least 4 unique of amino acids. In some embodiments, the oligopeptide backbone unit comprises at least 6 unique of amino acids. In some embodiments, the oligopeptide backbone unit comprises a secondary structural feature. In some embodiments, the secondary structural feature is stable at 60° C. In some embodiments, the secondary structural feature comprises an alpha-helix. In some embodiments, the oligopeptide backbone unit prevents contact between: (1) the functional handle, and (2) the first and the second fluorophores. In some embodiments, the oligopeptide backbone unit comprises a disulfide bond. In some embodiments, the oligopeptide backbone unit is configured to interconvert between a first state and a second state, and wherein a fluorescence intensity of the first fluorophore or the second fluorophore differs between the first state and the second state of the oligopeptide backbone unit. In some embodiments, the oligopeptide backbone unit comprises different conformations in the first state and the second state. In some embodiments, the interconverting between the first state and the second state comprises chemical interconversion. In some embodiments, the interconverting between the first state and the second state is temperature mediated, chemical condition mediated, or light mediated. In some embodiments, the oligopeptide backbone unit is linear. In some embodiments, the backbone unit is branched. In some embodiments, the backbone unit comprises a non-natural amino acid. In some embodiments, the backbone unit comprises a non-amino acid moiety. In some embodiments, the non-amino acid moiety is coupled to at least two amino acids of the backbone unit.

In some embodiments, an absorbance maximum of the first fluorophore and an absorbance maximum of the second fluorophore are within 10 nm. In some embodiments, an emission maximum of the first fluorophore and an emission maximum of the second fluorophore are within 10 nm. In some embodiments, the first fluorophore is identical to the second fluorophore. In some embodiments, the first fluorophore and the second fluorophore are separated by at most 20 Å. In some embodiments, the first fluorophore and the second fluorophore are separated by at most 12 Å. In some embodiments, a relative orientation of the first fluorophore and the second fluorophore is constrained by the backbone. In some embodiments, a dipole moment of the first fluorophore is substantially aligned with a dipole moment of the second fluorophore. In some embodiments, the first fluorophore is constrained from rotating relative to the second fluorophore. In some embodiments, the functional handle is inert towards the oligopeptide unit, the first fluorophore, and the second fluorophore. In some embodiments, the functional handle is configured to selectively couple to an amino acid type. In some embodiments, the amino acid type comprises cysteine, lysine, tyrosine, histidine, aspartic acid, glutamic acid, tryptophan, arginine, methionine, or any combination thereof. In some embodiments, the amino acid type comprises cysteine, and wherein the chemical moiety comprises an iodoacetamide, a thiol, a benzyl halide, an allyl halide, a selenocyanate, a maleimide, an alkyne, or any combination thereof. In some embodiments, the amino acid type comprises lysine, and wherein the chemical moiety comprises a thiocyanate, an isothiocyanate, a maleimide, an aldehyde, an isatoic anhydride, an NHS ester, or any combination thereof. In some embodiments, the amino acid type comprises aspartic acid or glutamic acid, and wherein the chemical moiety comprises an amine, an alcohol, a thiol, an organocuprate, or any combination thereof. In some embodiments, the amino acid type comprises tyrosine, and wherein the chemical moiety comprises a diazonium compound. In some embodiments, the amino acid type comprises histidine, and wherein the chemical moiety comprises an alpha-beta unsaturated carbonyl compound, an epoxide, or a combination thereof. In some embodiments, the amino acid type comprises arginine, and wherein the chemical moiety comprises an NHS ester. In some embodiments, the amino acid type comprises methionine, and wherein the chemical moiety comprises an oxaziridine compound. In some embodiments, the amino acid type comprises tryptophan, and wherein the chemical moiety comprises a diazopropanoate ester. In some embodiments, the amino acid type comprises a post-translationally modified amino acid type. In some embodiments, the post-translationally modified amino acid type comprises phosphorylation, glycosylation, nitrosylation, citrullination, sulfenylation, trimethylation, or any combination thereof. In some embodiments, the post-translationally modified amino acid type comprises phosphoserine, phosphotyrosine, phosphothreonine, or any combination thereof, and wherein the chemical moiety comprises a disulfide. In some embodiments, the amino acid type comprises an N-terminal amino acid or a C-terminal amino acid. In some embodiments, the functional handle is configured to couple to form a single attachment to the biomolecule. In some embodiments, the functional handle is configured to stoichiometrically couple to the biomolecule. In some embodiments, the detectable labeling reagent further comprising a triplet state quencher. In some embodiments, the first fluorophore or the second fluorophore comprises a plurality of fluorophores. In some embodiments, the first fluorophore and the second fluorophore each comprise a plurality of fluorophores. In some embodiments, the detectable labeling reagent (e.g., optically detectable labeling reagent) is inert towards Edman degradation. In some embodiments, the detectable labeling reagent (e.g., optically detectable labeling reagent) comprises an aqueous solubility of at least 2 mg/mL. In some embodiments, the detectable labeling reagent (e.g., optically detectable labeling reagent) comprises an aqueous solubility of at least 20 mg/mL.

In some embodiments, the first fluorophore comprises a diminished rate of photobleaching than the first fluorophore in the absence of the backbone unit, the second fluorophore, and the functional handle.

In another aspect, the present disclosure provides a system comprising a peptide, wherein the peptide comprises an amino acid type, and wherein each of a plurality of amino acids of the amino acid type are coupled a detectable labeling reagent disclosed herein. In some embodiments, the peptide is immobilized to a substrate. In some embodiments, the substrate comprises a bead, a polymer matrix, a surface, or a slide. In some embodiments, a C-terminus of the peptide is coupled to the substrate. In some embodiments, the system comprising the peptide further comprises a dye repellant. In some embodiments, a distance between the first fluorophore and the second fluorophore is greater than the distance in the absence of the dye-repellant. In some embodiments, the dye repellant comprises a counterion for the first fluorophore or the second fluorophore. In some embodiments, the dye repellant comprises an anion. In some embodiments, the dye repellant comprises a dicarboxylic acid compound. In some embodiments, the dicarboxylic acid compound comprises a benzenedicarboxylic acid. In some embodiments, a conformation of the peptide is substantially similar to a conformation of the peptide in the absence of the optically detectable labeling reagent. In some embodiments, a solubility of the peptide is similar to or greater than a solubility of the peptide in an absence of the optically detectable labeling reagent. In some embodiments, an intensity of a fluorescence signal of the detectable labeling reagent (e.g., optically detectable labeling reagent) is substantially linear with respect to the number of the plurality of amino acids coupled to the optically detectable labeling reagent. In some embodiments, two amino acids of the plurality of amino acids coupled to the detectable labeling reagent (e.g., optically detectable labeling reagent) are adjacent with respect to a sequence of the peptide. In some embodiments, an intensity of a fluorescence signal of the detectable labeling reagent (e.g., optically detectable labeling reagent) coupled to the two amino acids is approximately twice that of the detectable labeling reagent (e.g., optically detectable labeling reagent) in a dilute solution. In some embodiments, the peptide comprises a second amino acid type, and wherein a plurality of amino acids of the second amino acid type are coupled to a second detectable labeling reagent disclosed herein. In some embodiments, the first fluorophore and the second fluorophore of the detectable labeling reagent (e.g., optically detectable labeling reagent) are different than the first fluorophore and the second fluorophore of the second optically detectable labeling reagent. In some embodiments, the detectable labeling reagent (e.g., optically detectable labeling reagent) and the second detectable labeling reagent generate different fluorescence signals.

In another aspect, the present disclosure provides a method for analyzing a peptide, the method comprising: (a) coupling a labeling reagent to an amino acid of the peptide, wherein the labeling reagent comprises a backbone unit, a plurality of fluorophores, and a functional handle for the coupling to the amino acid of the peptide; (b) detecting a signal from the labeling reagent coupled to the peptide; and (c) using the signal to identify the amino acid of the peptide. In some embodiments, the functional handle comprises specificity for the amino acid of the peptide. In some embodiments, the amino acid comprises cysteine, lysine, tyrosine, histidine, aspartic acid, glutamic acid, tryptophan, arginine, methionine, N-terminal amino acids, C-terminal amino acids, or any combination thereof. In some embodiments, the amino acid comprises N-terminal amino acids or C-terminal amino acids. In some embodiments, the functional handle comprises pyridinecarboxyaldehyde (PCA). In some embodiments, the labeling reagent couples to the peptide with about 1:1 stoichiometry. In some embodiments, (c) further comprises quantifying a concentration or abundance of the peptide. In some embodiments, the concentration of the peptide is less than about 1 pM. In some embodiments, the amino acid comprises a plurality of amino acids, and wherein the labeling reagent couples to at least a subset of the plurality of amino acids. In some embodiments, the labeling reagent couples to each of the plurality of amino acids. In some embodiments, (c) comprises quantifying the plurality of amino acids of the peptide. In some embodiments, the method for analyzing a peptide further comprises (d) cleaving at least a portion of the peptide, and (e) detecting a signal or a signal change from the labeling reagent coupled to the peptide. In some embodiments, the method for analyzing a peptide further comprises (f) identifying a sequence of the peptide based at least in part on the signal or the signal change in (e). In some embodiments, the detecting comprises imaging the labeling reagent coupled to the peptide.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 provides a diagram of label components consistent with the present disclosure.

FIG. 2 provides a chemical structure of a peptide-based label consistent with the present disclosure.

FIG. 3 provides a chemical structure of an oligoethylene glycol label consistent with the present disclosure.

FIG. 4 provides an overview of a combined in-silico and experimental method for peptide-based label optimization.

FIGS. 5A-5B provide heat maps for libraries of labels related by single amino acid substitutions.

FIGS. 6A-6E summarize computational analyses of peptides with the sequence G-K-X₁-X₂-K-G (wherein X₁ and X₂ are varied between peptides; SEQ ID NO: 1). The x-axis for each plot provides calculation frame, with later frames corresponding to later structural optimization operations.

FIG. 6A summarizes inter-lysine backbone angles. The peptides with the largest angle deviation include: GKAQKG (SEQ ID NO: 2), GKHLKG (SEQ ID NO: 3), GKQVKG (SEQ ID NO: 4), GKLTKG (SEQ ID NO: 5), GKDTKG (SEQ ID NO: 6), GKNTKG (SEQ ID NO: 7), GKDIKG (SEQ ID NO: 8), GKRHKG (SEQ ID NO: 9), GKRTKG (SEQ ID NO: 10), GKNCKG (SEQ ID NO: 11), and GKHTKG (SEQ ID NO: 12). The peptides with the smallest angle deviation include: GKQPKG (SEQ ID NO: 13), GKREKG (SEQ ID NO: 14), GKMPKG (SEQ ID NO: 15), GKRRKG (SEQ ID NO: 16), GKEMKG (SEQ ID NO: 17), GKNIKG (SEQ ID NO: 18), GKIPKG (SEQ ID NO: 19), GKAEKG (SEQ ID NO: 20), GKCEKG (SEQ ID NO: 21), GKRMKG (SEQ ID NO: 22), and GKEPKG (SEQ ID NO: 23). The sequences along the right side of the graph represent, from top to bottom, SEQ ID NOs: 2, 13, 14, 15, 3, 16, 4, 5, 6, 7, 17, 18, 32, 9, 10, 11, 43, 20, 21, 22, 23, and 33, respectively.

FIG. 6B summarizes the distance between lysine side chain amines (NZ atom distance). The peptides with the largest NZ distance include: GKASKG (SEQ ID NO: 24), GKPSKG (SEQ ID NO: 25), GKDLKG (SEQ ID NO: 26), GKSVKG (SEQ ID NO: 27), GKRHKG (SEQ ID NO: 28), GKDTKG (SEQ ID NO: 29), GKMVKG (SEQ ID NO: 30), GKGVKG (SEQ ID NO: 31), GKDIKG (SEQ ID NO: 32), and GKHTKG (SEQ ID NO: 33). The peptides with the smallest NZ distance include: GKAPKG (SEQ ID NO: 34), GKEPKG (SEQ ID NO: 35), GKNIKG (SEQ ID NO: 36), GKCEKG (SEQ ID NO: 37), GKRPKG (SEQ ID NO: 38), GKDQKG (SEQ ID NO: 39), GKLSKG (SEQ ID NO: 40), GKNQKG (SEQ ID NO: 41), and GKRRKG (SEQ ID NO: 42). The sequences along the right side of the graph represent, from top to bottom, SEQ ID NOs: 24, 34, 35, 36, 37, 25, 26, 27, 38, 28, 6, 30, 39, 40, 31, 41, 8, 33, and 16, respectively.

FIG. 6C summarizes peptide potential energy. The peptides with the largest potential energy include: GKIPKG (SEQ ID NO: 43), GKCGKG (SEQ ID NO: 44), GKILKG (SEQ ID NO: 45), GKLMKG (SEQ ID NO: 46), GKCMKG (SEQ ID NO: 47), GKIMKG (SEQ ID NO: 48), GKMMKG (SEQ ID NO: 49), GKIIKG (SEQ ID NO: 50), GKCHKG (SEQ ID NO: 51), and GKNIKG (SEQ ID NO: 52). The peptides with the smallest potential energy include: GKRSKG (SEQ ID NO: 53), GKDLKG (SEQ ID NO: 54), GKRQKG (SEQ ID NO: 55), GKRRKG (SEQ ID NO: 56), GKDEKG (SEQ ID NO: 57), GKRDKG (SEQ ID NO: 58), GKADKG (SEQ ID NO: 59), GKNDKG (SEQ ID NO: 60), GKDDKG (SEQ ID NO: 61), and GKRMKG (SEQ ID NO: 62). The sequences along the right side of the graph represent, from top to bottom, SEQ ID NOs: 43, 53, 44, 45, 54, 46, 55, 47, 48, 42, 49, 50, 57, 58, 59, 60, 51, 61, and 52, respectively.

FIG. 6D summarizes peptide kinetic energy. The peptides with the largest kinetic energy include: GKRNKG (SEQ ID NO: 63), GKRQKG (SEQ ID NO: 64), GKRGKG (SEQ ID NO: 65), GKRMKG (SEQ ID NO: 66), GKRLKG (SEQ ID NO: 67), GKRPKG (SEQ ID NO: 68), GKRRKG (SEQ ID NO: 69), GKRCKG (SEQ ID NO: 70), GKREKG (SEQ ID NO: 71), GKRHKG (SEQ ID NO: 72), and GKRIKG (SEQ ID NO: 73). The peptides with the smallest kinetic energy include: GKGGKG (SEQ ID NO: 74), GKATKG (SEQ ID NO: 75), GKSTKG (SEQ ID NO: 76), GKGSKG (SEQ ID NO: 77), GKASKG (SEQ ID NO: 78), GKADKG (SEQ ID NO: 79), GKSSKG (SEQ ID NO: 80), GKGTKG (SEQ ID NO: 81), GKTTKG (SEQ ID NO: 82), and GKGVKG (SEQ ID NO: 83). The sequences along the right side of the graph represent, from top to bottom, SEQ ID NOs: 126, 118, 140, 141, 134, 135, 142, 121, 143, 136, 137, 144, 145, 112, 147, 142, 138, and 139, respectively.

FIG. 6E summarizes peptide root-mean-square distributions (RMSDs). The peptides with the largest RMSD include: GKGGKG (SEQ ID NO: 84), GKATKG (SEQ ID NO: 85), GKSTKG (SEQ ID NO: 86), GKGSKG (SEQ ID NO: 87), GKASKG (SEQ ID NO: 88), GKADKG (SEQ ID NO: 89), GKSSKG (SEQ ID NO: 90), GKGTKG (SEQ ID NO: 91), GKTTKG (SEQ ID NO: 92), and GKGVKG (SEQ ID NO: 93). The peptides with the smallest RMSD include: GKGGKG (SEQ ID NO: 94), GKATKG (SEQ ID NO: 95), GKSTKG (SEQ ID NO: 96), GKGSKG (SEQ ID NO: 97), GKASKG (SEQ ID NO: 98), GKADKG (SEQ ID NO: 99), GKSSKG (SEQ ID NO: 100), GKGTKG (SEQ ID NO: 101), GKTTKG (SEQ ID NO: 102), and GKGVKG (SEQ ID NO: 103). The sequences along the right side of the graph represent, from top to bottom, SEQ ID NOs: 193, 41, 74, and 194-209, respectively.

FIGS. 7A-7E summarize computational analyses of peptides with the sequence G-K-X₁-K-X₂-G (wherein X₁ and X₂ are varied between peptides; SEQ ID NO: 104). The x-axis for each plot provides calculation frame, with later frames corresponding to later structural optimization operations.

FIG. 7A summarizes inter-lysine backbone angles. The peptides with the largest angle deviation include: GKGGKG (SEQ ID NO: 74), GKATKG (SEQ ID NO: 75), GKSTKG (SEQ ID NO: 76), GKGSKG (SEQ ID NO: 77), GKASKG (SEQ ID NO: 78), GKADKG (SEQ ID NO: 79), GKSSKG (SEQ ID NO: 80), GKGTKG (SEQ ID NO: 81), GKTTKG (SEQ ID NO: 82), and GKGVKG (SEQ ID NO: 83). The peptides with the smallest angle deviation include: GKPKQG (SEQ ID NO: 105), GKLKDG (SEQ ID NO: 107), GKIKQG (SEQ ID NO: 108), GKHKDG (SEQ ID NO: 109), GKPKDG (SEQ ID NO: 110), GKPKEG (SEQ ID NO: 111), GKGKDG (SEQ ID NO: 112), GKEKMG (SEQ ID NO: 113), and GKQKDG (SEQ ID NO: 114). The sequences along the right side of the graph represent, from top to bottom, SEQ ID NOs: 210, 105, and 211-226, respectively.

FIG. 7B summarizes the distance between lysine side chain amines (NZ atom distance). The peptides with the largest NZ distance include: GKPKQG (SEQ ID NO: 115), GKLKDG (SEQ ID NO: 107), GKIKQG (SEQ ID NO: 108), GKHKDG (SEQ ID NO: 109), GKPKDG (SEQ ID NO: 110), GKPKEG (SEQ ID NO: 111), GKGKDG (SEQ ID NO: 112), GKEKMG (SEQ ID NO: 113), and GKQKDG (SEQ ID NO: 114). The peptides with the smallest NZ distance include: GKPKQG (SEQ ID NO: 115), GKLKDG (SEQ ID NO: 107), GKIKQG (SEQ ID NO: 108), GKHKDG (SEQ ID NO: 109), GKPKDG (SEQ ID NO: 110), GKPKEG (SEQ ID NO: 111), GKGKDG (SEQ ID NO: 112), GKEKMG (SEQ ID NO: 113), and GKQKDG (SEQ ID NO: 114). The sequences along the right side of the graph represent, from top to bottom, SEQ ID NOs: 227-229, 212, 230-234, 222, and 235-244, respectively.

FIG. 7C summarizes peptide potential energy. The peptides with the largest potential energy include: GKCKHG (SEQ ID NO: 116), GKHKHG (SEQ ID NO: 117), GKMKHG (SEQ ID NO: 118), GKVKHG (SEQ ID NO: 119), GKMKMG (SEQ ID NO: 120), GKIKHG (SEQ ID NO: 121), GKMKAG (SEQ ID NO: 122), GKPKHG (SEQ ID NO: 123), and GKQKLG (SEQ ID NO: 124). The peptides with the smallest potential energy include: GKGKCG (SEQ ID NO: 125), GKGKMG (SEQ ID NO: 126), GKTKRG (SEQ ID NO: 127), GKDKNG (SEQ ID NO: 128), GKTKDG (SEQ ID NO: 129), GKDKDG (SEQ ID NO: 130), GKSKCG (SEQ ID NO: 131), GKGKDG (SEQ ID NO: 112), GKDKRG (SEQ ID NO: 132), and GKEKRG (SEQ ID NO: 133). The sequences along the right side of the graph represent, from top to bottom, SEQ ID NOs: 116, 125, 117, 126, 127, 118, 128, 129, 119, 120, 121, 130, 131, 122, 245, 112, 123, 132, 133, and 124, respectively.

FIG. 7D summarizes peptide kinetic energy. The peptides with the largest kinetic energy include: GKMKHG (SEQ ID NO: 118), GKEKHG (SEQ ID NO: 134), GKMKLG (SEQ ID NO: 135), GKIKHG (SEQ ID NO: 121), GKDKHG (SEQ ID NO: 136), GKQKMG (SEQ ID NO: 137), GKQKLG (SEQ ID NO: 124), GKQKHG (SEQ ID NO: 138), and GKQKVG (SEQ ID NO: 139). The peptides with the smallest kinetic energy include: GKGKMG (SEQ ID NO: 126), GKGKPG (SEQ ID NO: 140), GKGKIG (SEQ ID NO: 141), GKGKEG (SEQ ID NO: 142), GKGKQG (SEQ ID NO: 143), GKGKRG (SEQ ID NO: 144), GKGKVG (SEQ ID NO: 145), GKGTDG (SEQ ID NO: 146), and GKGKSG (SEQ ID NO: 147). The sequences along the right side of the graph represent, from top to bottom, SEQ ID NOs: 126, 118, 140, 141, 134, 135, 142, 121, 143, 136, 137, 144, 145, 224, 147, 124, 138 and 139, respectively.

FIG. 7E summarizes peptide root-mean-square distributions (RMSDs). The peptides with the largest RMSD include: GKGKMG (SEQ ID NO: 126), GKGKPG (SEQ ID NO: 140), GKGKIG (SEQ ID NO: 141), GKGKEG (SEQ ID NO: 142), GKGKQG (SEQ ID NO: 143), GKGKRG (SEQ ID NO: 144), GKGKVG (SEQ ID NO: 145), GKGTDG (SEQ ID NO: 146), and GKGKSG (SEQ ID NO: 147). The peptides with the smallest RMSD include GKQKAG (SEQ ID NO: 148), GKCKPG (SEQ ID NO: 149), GKPKIG (SEQ ID NO: 150), GKPKPG (SEQ ID NO: 151), GKSKVG (SEQ ID NO: 152), GKMKVG (SEQ ID NO: 153), GKGKDG (SEQ ID NO: 130), GKTKIG (SEQ ID NO: 154), GKMKIG (SEQ ID NO: 155), and GKCKDG (SEQ ID NO: 156). The sequences along the right side of the graph represent, from top to bottom, SEQ ID NOs: 228, 246, 148, 149, 126, 247, 150, 151, 236, 144, 223, 152, 245, 153, 112, 154, 155, 241, 156, and 244, respectively.

FIGS. 8A-8E summarize computational analyses of peptides with the sequence G-K—R-X-R-K-G (SEQ ID NO: 157) and G-K-D-X-I-K-G (SEQ ID NO: 158) (wherein X is varied between individual peptides). The x-axis for each plot provides calculation frame, with later frames corresponding to later structural optimization operations.

FIG. 8A summarizes inter-lysine backbone angles. The peptides with the largest angle deviation include: GKRWRKG (SEQ ID NO: 159), GKRARKG (SEQ ID NO: 160), GKRMRKG (SEQ ID NO: 161), GKRQRKG (SEQ ID NO: 162), GKRTRKG (SEQ ID NO: 163), GKRIRKG (SEQ ID NO: 164), GKRERKG (SEQ ID NO: 165), GKDVIKG (SEQ ID NO: 166), GKRNRKG (SEQ ID NO: 167), and GKRHRKG (SEQ ID NO: 168). The peptides with the smallest angle deviation include: GKRWRKG (SEQ ID NO: 159), GKRARKG (SEQ ID NO: 160), GKRMRKG (SEQ ID NO: 161), GKRQRKG (SEQ ID NO: 162), GKRTRKG (SEQ ID NO: 163), GKRIRKG (SEQ ID NO: 164), GKRERKG (SEQ ID NO: 165), GKDVIKG (SEQ ID NO: 166), GKRNRKG (SEQ ID NO: 167), and GKRHRKG (SEQ ID NO: 168). The sequences along the right side of the graph represent, from top to bottom, SEQ ID NOs: 170, 171, 159, 172, 173, 160, 161, 162, 174, 163, 248, 164, 187, 165, 166, 167, 168, 182, and 183, respectively.

FIG. 8B summarizes the distance between lysine side chain amines (NZ atom distance). The peptides with the largest NZ distance deviation include: GKRWRKG (SEQ ID NO: 159), GKRARKG (SEQ ID NO: 160), GKRMRKG (SEQ ID NO: 161), GKRQRKG (SEQ ID NO: 162), GKRTRKG (SEQ ID NO: 163), GKRIRKG (SEQ ID NO: 164), GKRERKG (SEQ ID NO: 165), GKDVIKG (SEQ ID NO: 166), GKRNRKG (SEQ ID NO: 167), and GKRHRKG (SEQ ID NO: 168). The peptides with the smallest NZ distance deviation include: GKDNIKG (SEQ ID NO: 169), GKDFIKG (SEQ ID NO: 170), GKDHIKG (SEQ ID NO: 171), GKRRRKG (SEQ ID NO: 172), GKDEIKG (SEQ ID NO: 173), GKDPIKG (SEQ ID NO: 174), GKDTIKG (SEQ ID NO: 175), GKDSIKG (SEQ ID NO: 176), and GKDWIKG (SEQ ID NO: 177). The sequences along the right side of the graph represent, from top to bottom, SEQ ID NOs: 169, 170, 171, 159, 172, 173, 160, 161, 174, 163, 175, 185, 167, 168, 182, 249, 176, and 177, respectively.

FIG. 8C summarizes peptide potential energy. The peptides with the largest potential energy include: GKDNIKG (SEQ ID NO: 169), GKDFIKG (SEQ ID NO: 170), GKDHIKG (SEQ ID NO: 171), GKRRRKG (SEQ ID NO: 172), GKDEIKG (SEQ ID NO: 173), GKDPIKG (SEQ ID NO: 174), GKDTIKG (SEQ ID NO: 175), GKDSIKG (SEQ ID NO: 176), and GKDWIKG (SEQ ID NO: 177). The peptides with the smallest potential energy include: GKDDIKG (SEQ ID NO: 178), GKRSRKG (SEQ ID NO: 179), GKRQRKG (SEQ ID NO: 162), GKRTRKG (SEQ ID NO: 163), GKRERKG (SEQ ID NO: 165), GKRFRKG (SEQ ID NO: 180), GKRVRKG (SEQ ID NO: 181), GKRDRKG (SEQ ID NO: 182), and GKDRIKG (SEQ ID NO: 183). The sequences along the right side of the graph represent, from top to bottom, SEQ ID NOs: 170, 178, 159, 161, 179, 162, 174, 163, 250, 248, 164, 165, 180, 181, 182, 188, 183, and 177, respectively.

FIG. 8D summarizes peptide kinetic energy. The peptides with the largest kinetic energy include: GKRWRKG (SEQ ID NO: 159), GKRRRKG (SEQ ID NO: 172), GKRMRKG (SEQ ID NO: 161), GKRQRKG (SEQ ID NO: 162), GKRLRKG (SEQ ID NO: 184), GKRYRKG (SEQ ID NO: 185), GKRIRKG (SEQ ID NO: 164), GKRERKG (SEQ ID NO: 165), and GKRNRKG (SEQ ID NO: 167). The peptides with the smallest kinetic energy include: GKDFIKG (SEQ ID NO: 170), GKDDIKG (SEQ ID NO: 178), GKDHIKG (SEQ ID NO: 171), GKDTIKG (SEQ ID NO: 175), GKAIKG (SEQ ID NO: 186), GKDCIKG (SEQ ID NO: 187), GKDVIKG (SEQ ID NO: 166), GKDYIKG (SEQ ID NO: 167), GKDGIKG (SEQ ID NO: 189), and GKDSIKG (SEQ ID NO: 176). The sequences along the right side of the graph represent, from top to bottom, SEQ ID NOs: 170, 178, 171, 159, 172, 162, 184, 175, 185, 248, 164, 187, 165, 166, 167, 188, 189, and 176, respectively.

FIG. 8E summarizes peptide root-mean-square distributions (RMSDs). The peptides with the largest RMSD include: GKDFIKG (SEQ ID NO: 170), GKDDIKG (SEQ ID NO: 178), GKDHIKG (SEQ ID NO: 171), GKDTIKG (SEQ ID NO: 175), GKAIKG (SEQ ID NO: 186), GKDCIKG (SEQ ID NO: 187), GKDVIKG (SEQ ID NO: 166), GKDYIKG (SEQ ID NO: 188), GKDGIKG (SEQ ID NO: 189), and GKDSIKG (SEQ ID NO: 176). The peptides with the smallest RMSD include: GKDFIKG (SEQ ID NO: 170), GKDDIKG (SEQ ID NO: 178), GKDHIKG (SEQ ID NO: 171), GKDTIKG (SEQ ID NO: 175), GKAIKG (SEQ ID NO: 186), GKDCIKG (SEQ ID NO: 187), GKDVIKG (SEQ ID NO: 166), GKDYIKG (SEQ ID NO: 188), GKDGIKG (SEQ ID NO: 189), and GKDSIKG (SEQ ID NO: 176). The sequences along the right side of the graph represent, from top to bottom, SEQ ID NOs: 178, 171, 159, 173, 160, 174, 163, 250, 175, 185, 248, 251, 180, 167, 168, 182, 189, and 177, respectively.

FIGS. 9A-9D provide predicted fluorophore signal intensities for select peptides over structure optimization operations.

FIG. 10 provides a chemical structure of a dye repellant consistent with the present disclosure.

FIG. 11 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “analyte” or “analytes,” as used herein, generally refers to a molecule whose presence or absence is measured or identified. An analyte can be a molecule for which a detectable probe or assay exists or can be produced. For example, an analyte can be a macromolecule, such as, for example, a nucleic acid, a polypeptide, a carbohydrate, a small organic, an inorganic compound, or an element, for example, gold, iron, or lead. An analyte can be part of a sample that contains other components or can be the sole or the major component of the sample. An analyte can be a component of a whole cell or tissue, a cell or tissue extract, a fractionated lysate thereof or a substantially purified molecule. In some embodiments, the target analyte is a polypeptide.

The terms “polypeptide” and “peptide” generally to refer to a polymer of amino acids in which an amino acid may be linked to another amino acid by a peptide bond. In some examples, a polypeptide is a protein. The amino acid may be a naturally occurring amino acid or a non-naturally occurring amino acid (e.g., amino acid analogue). The polymer can be linear or branched and can include modified amino acids, and/or may be interrupted by non-amino acids. Polypeptides can occur as single chains or associated chains. The polymer may include a plurality of amino acids and may have a secondary and tertiary structure (e.g., protein). In some examples, the polymer comprises at least 2, 3, 4, 5, 6, 7, 8, 9, 10, 20, 30, 40, 50, 100, 1000, 10,000, or more amino acids.

The term “amino acid,” as used herein, generally refers to a naturally occurring or non-naturally occurring amino acid (amino acid analogue). The non-naturally occurring amino acid may be a synthesized amino acid. As used herein, the terms “amino acid sequence,” “peptide sequence,” and “polypeptide sequence,” as used herein, generally refer to at least two amino acids or amino acid analogs that are covalently linked by a peptide (amide) bond or an analog of a peptide bond. The term peptide includes oligomers and polymers of amino acids or amino acid analogs. The amino acids of the peptide may be L-amino acids or D-amino acids. A peptide, polypeptide, or protein may be synthetic, recombinant, or naturally occurring. A synthetic peptide may be a peptide that is produced by artificial approaches in vitro.

The terms “amino acid sequence,” “peptide sequence,” and “polypeptide sequence,” as used herein, generally refer to a sequence of at least two amino acids or amino acid analogs that are covalently linked (e.g., by a peptide (amide) bond or an analog of a peptide bond). A peptide sequence may refer to a complete sequence or a portion of a sequence. For example, a peptide sequence may contain gaps, positions with unknown identities, or positions that can accommodate distinct species.

As used herein, the term “side chain” or “R-group” generally refers to structures attached to an amino acid alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−), and glutamate (−): amino acids can also be basic, such as lysine, or acidic, such as glutamic acid: uncharged polar side chains have hydroxyl, amide, or thiol groups, such as cysteine having a chemically reactive side chain, e.g., a thiol group that can form bonds with another cysteine, serine (Ser) and threonine (Thr), that have hydroxylic R side chains of different sizes; asparagine (Asn), glutamine (Gln), and tyrosine (Tyr); non-polar hydrophobic amino acid side chains include the amino acid glycine, alanine, valine, leucine, and isoleucine having aliphatic hydrocarbon side chains ranging in size from a methyl group for alanine to isomeric butyl groups for leucine and isoleucine; methionine (Met) has a thiol ether side chain; proline (Pro) has a cyclic pyrrolidine side group. Phenylalanine (with its phenyl moiety) (Phe) and tryptophan (Trp) (with its indole group) contain aromatic side chains, which are characterized by bulk as well as lack of polarity.

The term “cleavable unit,” as used herein, generally refers to a molecule that can be split into at least two molecules. Non-limiting examples of cleavage reagents and conditions to split a cleavable unit include: enzymes, nucleophilic or basic reagents, reducing agents, photo-irradiation, electrophilic or acidic reagents, organometallic or metal reagents, and oxidizing reagents.

The term “sample,” as used herein, generally refers to a sample containing or suspected of containing a polypeptide. For example, a sample can be a biological sample containing one or more polypeptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the polypeptides and/or to include reagents.

As used herein, the term “support” generally refers to an entity to which a substance (e.g., molecular construct) can be coupled, immobilized, or adsorbed. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, a lantern, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane. The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine, or asparagine). The functional group may couple specifically to a reactive group on a particular species, such as a label. In some examples of functionalized beads, the functional motif can be reversibly coupled and cleaved. A functional motif can also irreversibly couple to a molecule.

One such type of substrate is a lantern, which may comprise a solid support comprising peptide capture agents, and a rod for positioning the solid support within a sample. A lantern rod may be manipulatable by a user (e.g., the user may hold the lantern rod) or an instrument. A lantern rod may be configured to connect to a member proximal to a sample volume. For example, a lantern rod may be configured to couple to a clip above a well of a well plate. A lantern solid support may comprise a reactive group of the present disclosure, such as a reactive group selective for cysteine or a peptide C-terminus. A lantern may be dried or frozen with peptides coupled to its solid support, which may stabilize the peptides coupled thereto. Unbound peptide may be washed from a lantern solid support.

As used herein, sequencing of peptides “at the single molecule level” generally refers to amino acid sequence information obtained from individual (e.g., single) peptide molecules in a mixture of diverse peptide molecules. The amino acid sequence information may be obtained from an entirety of an individual peptide molecule or one or more portion of the individual peptide molecule, such as a contiguous amino acid sequence of at least a portion of the individual peptide molecule. Alternatively, partial amino acid sequence information may be obtained, which may allow for identification of the peptide or protein. Partial amino acid sequence information, including for example, the pattern of a specific amino acid residue (e.g., lysine) within individual peptide molecules, may be sufficient to uniquely identify an individual peptide molecule. For example, a pattern of amino acids may comprise a plurality of identified positions (e.g., identified as a particular amino acid type, such as lysine, or identified as a particular set of amino acids, such as the set of carboxylate side chain-containing amino acids), and a plurality of unidentified positions. The sequence of identified positions may be searched against a known proteome of a given organism to identify the individual peptide molecule. In some examples, sequencing of a peptide at the single molecule level may identify a pattern of a certain type of amino acid (e.g., lysine) in an individual peptide molecule. Such information may be used to identify a macromolecule (e.g., protein) from which the peptide was derived. This may advantageously preclude identifying all amino acids of the peptide.

As used herein, the term “Edman degradation” generally refers to methods comprising chemical removal of amino acids from peptides or proteins. In some cases, Edman degradation denotes terminal (e.g., N- or C-terminal) amino acid removal. In specific cases, Edman degradation refers to N-terminal amino acid removal through isothiocyanate (e.g., phenyl isothiocyanate) coupling and cyclization with the terminal amine group of an N-terminal residue, such that the N-terminal amino acid is removed from a peptide. In some cases, Edman degradation broadly encompasses N-terminal amino acid functionalizations leading to N-terminal amino acid removal. In some cases, Edman degradation encompasses C-terminal amino acid removal. In some cases, Edman degradation comprises a catalyst (e.g., an enzyme) for amino acid functionalization or removal. For example, Edman degradation may comprise terminal amino acid functionalization (e.g., N-terminal amino acid isothiocyanate functionalization) followed enzyme-mediated removal of the functionalized terminal amino acid from the peptide (e.g., by an ‘Edmanase’ with specificity for chemically derivatized N-terminal amino acids).

As used herein, the term “single molecule sensitivity” generally refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). This may include the ability to simultaneously record the fluorescent intensity of multiple individual (e.g., single) peptide molecules distributed across the glass surface. Optical devices are commercially available that can be applied in this manner. For example, a microscope equipped with total internal reflection illumination and an intensified charge-couple device (CCD) detector is available. Imaging with a high sensitivity CCD camera allows the instrument to simultaneously record the fluorescent intensity of multiple individual (e.g., single) peptide molecules distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single polypeptide having a particular sequence or a site can include several polypeptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group, such as an amino acid side chain, C-terminal carboxylate, or N-terminal amine. The label may comprise at least one reactive group (e.g., a first reactive group and a second reactive group). The at least one reactive group may be configured to couple to a polypeptide. The at least one reactive group may be configured to couple to a support. The at least one reactive group may be coupled to or configured to couple to a detectable moiety. A label may provide a measurable signal.

As used herein, the term “polymer matrix” generally refers to a continuous phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid or gaseous species. For example, the term ‘polymer matrix’ may encompass desiccated hydrogels, hydrated hydrogels, and hydrogels containing glass fibers.

Peptide sequence information may be obtained from a polypeptide molecule or from one or more portions of the polypeptide molecule. Peptide sequencing may provide complete or partial amino acid sequence information for a peptide sequence or a portion of a peptide sequence. At least a portion of the peptide sequence may be determined at the single molecule level. In some cases, partial amino acid sequence information, including for example, the relative positions of a specific type of amino acid (e.g., lysine) within a peptide or portion of a peptide, may be sufficient to uniquely identify an individual peptide molecule. For example, a pattern of amino acids, such as, for example, X-X-X-Lys-X-X-X-X-Lys-X-Lys, which indicates the distribution of lysine molecules within an individual peptide molecule, may be searched against a known proteome of a given organism to identify the individual peptide molecule. Such information may be used to identify a macromolecule (e.g., protein) from which the peptide was derived, and may preclude identifying all amino acids of the peptide. Peptide sequencing may be used to acquire information (including, for example, amino acid sequence information) from individual peptide molecules in a mixture of diverse peptide molecules. In a non-limiting example, a plurality of peptides may be immobilized on a solid surface (including, for example, a glass slide, or a glass slide whose surface has been chemically modified, a plastic slide, a multi-well plate, a cassette), amino acids from the plurality of peptides may be coupled to fluorescent reporter moieties, and the fluorescent reporter moieties may be optically detected.

Numerous commercially available optical devices can be applied in this manner. For example, microscopes equipped with total internal reflection illumination and intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands or more (e.g., millions) of individual single peptides (or more) to be sequenced in a single experiment.

In an aspect, the present disclosure provides solutions to the aforementioned challenges by providing expeditious and facile methods for analyzing polypeptides. Aspects of the present disclosure provide labels configured for improved molecular and sub-molecular discrimination and diminished signal aberrations. Additionally, some aspects of the present disclosure provide compositions that facilitate effective peptide characterization and analysis. Furthermore, in some aspects the present disclosure provides kits which enable effective polypeptide analysis.

### Fluorosequencing

A characteristic feature of many fluorosequencing methods is coupling amino acid labels to a peptide to be sequenced. A label may be configured to couple to a specific type of amino acid or a specific number of unique amino acids. A fluorosequencing method may comprise labeling a plurality of unique amino acids with separate, amino acid type specific labels. A fluorosequencing method may comprise labeling one, two, three, four, five, six, or more unique amino acids residues in a subject peptide or protein. A peptide may comprise a label on an N-terminal amino acid, cysteine, lysine, glutamic acid, aspartic acid, tryptophan, tyrosine, serine, threonine, arginine, histidine, methionine, or any combination thereof. A peptide may comprise a label on a non-canonical amino acid, such as a phosphoserine/phosphothreonine, pyroglutamic acid, hydroxyproline, azidolysine, dehydroalanine, or any combination thereof. Each of these amino acid residues may be labeled with a different labels. Multiple amino acid residues may be labeled with the same label such as (i) aspartic acid and glutamic acid or (ii) serine and threonine.

A label may comprise a detectable moiety. The detectable moiety may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different detectable moiety that uniquely identifies the label by its type.

A label may reversibly or irreversibly bind to an amino acid type, and thus may be chemically (e.g., by addition of a cleavage reagent) or physically (e.g., by addition of heat or light) decoupled from a target peptide. A method may thus comprise blocking a first amino acid, labeling a second amino acid type (e.g., threonine), unblocking the first amino acid type, and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

Labeling specificity can be a major challenge for a fluorosequencing method. In many cases, a label may comprise reactivity toward a plurality of unique amino acid identities. For example, some maleimide labels can react with cysteine, lysine, and N-terminal amines. A number of strategies may be employed to utilize or prevent such cross-reactivity. A method may comprise sequential amino acid labeling, for example to ensure that a multi-specific label is added to a system after one or more unique amino acid identities with which the multi-specific label is configured to couple are chemically blocked or labeled, and therefore unable to react with the multi-specific label.

Fluorosequencing may comprise removing peptides through techniques such as chemical cleavage, Edman degradation, or other forms of enzymatic cleavage following or preceding subject peptide detection. Sequential peptide removal may generate sequence or position-specific information. For example, a reduction in fluorescence following an N-terminal amino acid removal operation may indicate that a labeled amino acid, and thus that a specific type of amino acid, was disposed at a peptide N-terminal. Removal of each amino acid residue can be carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

A label, detectable moiety, or protecting group of the present disclosure may be configured to withstand conditions for removing one or more of amino acid residues from a peptide. Some non-limiting examples of potential reporter moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5) 6-napthofluorescein. A detectable moiety may comprise fluorescent peptide (e.g., green fluorescent protein or a variant thereof) or an optically detectable material, such as a carbon nanotube, a nanorod, or a quantum dot.

Peptide detection or imaging may comprise immobilizing the peptide on a surface. The peptide may be immobilized to the surface by coupling a peptide-derived cysteine residue, the peptide N-terminus, or the peptide C-terminus with the surface or with a reagent coupled to the surface. The peptide may be immobilized by reacting the cysteine residue with the surface or with a capture reagent coupled to the surface. The peptide may be immobilized by coupling the peptide C-terminus or N-terminus with a capture moiety described herein. The peptide may be immobilized on a surface. Detecting the immobilized peptide may comprise capturing an image comprising the peptide. The image may comprise a spatial address specific to the peptide. A plurality of peptides may be detected in a single imagine, wherein one or more of the peptides may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labels or reporter moieties (e.g., amino acid labels) coupled to the peptide. The sequencing technique may comprise imaging a plurality of peptides or proteins to determine the presence of one or more labels or reporter moieties on individual peptides from among the plurality of peptides. The sequencing technique may comprise imaging at least 103, at least 104, at least 105, at least 106, at least 107, at least 108 or more proteins or peptides (e.g., imaging a portion of a surface comprising at least 103 to at least 108 proteins or peptides). These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (wherein ‘K’ denotes lysine, ‘D’ denotes aspartate, ‘Y’ denotes tyrosine, ‘A’ denotes alanine, and ‘G’ denotes glycine; SEQ ID NO: 190), and may comprise labels coupled to each lysine and tyrosine residue. A first image comprising the C-terminal immobilized peptide may indicate the presence of two lysines and one tyrosine in the peptide. The N-terminal amino acid may be removed (e.g., by Edman degradation), such that a second image comprising the C-terminal immobilized peptide may indicate the presence of one lysine and one tyrosine in the peptide. This process may be repeated until a sequence of KXXYXXXXXXXKX is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and comparing these locations to known peptide sequences, which may identify the entire list of amino acid residues in the peptide sequence. For example, identifying the positions of the lysines and cysteines in a 40 amino acid fragment of a human protein may uniquely identify the protein (e.g., only one human protein contains the specific pattern of lysine and cysteine residues identified in the 40 amino acid fragment).

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and imaging a subject peptide, the position of the labeled amino acid residue can be determined in the peptide.

### Detectable Labels

Many labeling assays require high label densities to deconvolute structural and positional information. While detection techniques continue to advance past diffraction limited resolutions, few labels are amenable to close packing at the molecular level required for many forms of analysis. In many cases, signal intensity, signal types (e.g., the wavelength of an optical signal or the potential of a reduction or oxidation potential), and signal stability comprise dependencies on distance and relative orientation between labels in a sample. For example, many fluorescent dyes quench over multi-nanometer distances, rendering single molecule multi-labeling experiments inaccurate and sometimes infeasible.

The present disclosure provides a range of labeling reagents which maintain stable signal intensities and resist signal aberrations (e.g., fluorophore quenching and chromatic shifts) from inter-label interactions. Such labels may generate quantifiable signal intensities in high label density systems, such as fluorosequencing assays in which consecutive amino acids are labeled. The labels may also comprise site and chemical coupling specificity for selective single- and multi-labeling assays.

A label consistent with the present disclosure may comprise: (i) a chemical handle for selectively coupling to a target species, (ii) a backbone unit, and (iii) a detectable moiety. Compared to direct attachment of the detectable moiety to the target species, the backbone unit may space and orient the detectable moiety relative to the target species such that interactions with detectable moieties of other labels coupled to or near the target species are minimized. The backbone unit may be covalently coupled to the chemical handle and the detectable moiety. The chemical handle may comprise a plurality of chemical handles. The detectable moiety may comprise a plurality of detectable moieties. For example, a label consistent with the present disclosure may comprise two chemical handles configured to couple to different N-glycosylation protein sites and three different color fluorophores, each coupled to different sites along a single peptide backbone. The label may further comprise a species which protects a detectable moiety from photo or chemical damage, such as a triplet-state quencher.

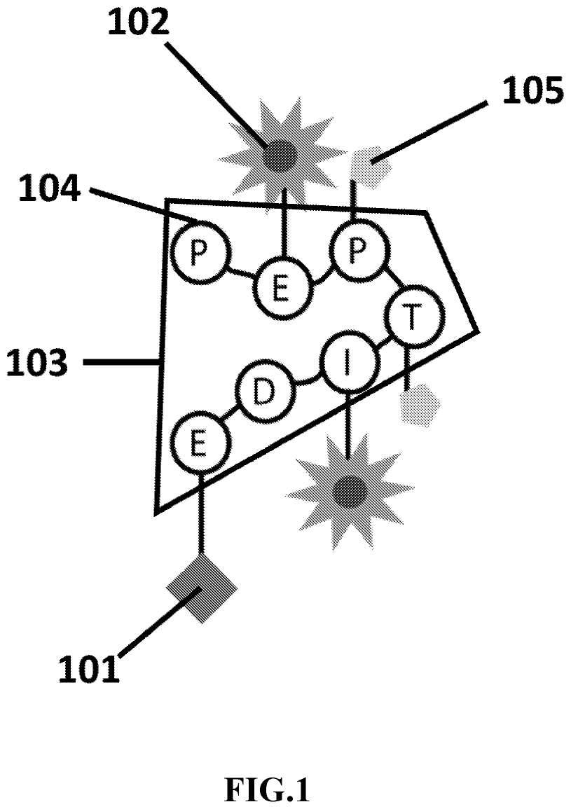

FIG. 1 provides a diagram of a label consistent with the present disclosure. The label may comprise a chemical handle 101 for coupling to a target species, a detectable moiety 102 configured to generate a detectable signal, and a backbone unit 103. The backbone unit 103 may comprise a polymer or oligomer comprised of individual subunits 104. For example, the backbone unit 103 may be an oligopeptide comprised of individual amino acid residues. The backbone unit may comprise a conformation, such as a peptidic secondary or tertiary structure. The conformation may comprise a condition dependence, or may be switchable between conformations upon chemical or physical treatment (e.g., reduction or irradiation). The chemical handle 101 and detectable moiety 102 may be coupled to the backbone unit. For example, each instance of a detectable moiety and a chemical handle may be coupled to separate amino acid residues of a peptide backbone unit 103. The label may comprise multiple detectable moieties. The label may also comprise species configured to stabilize detectable moieties 105, such as triplet-state quenchers for preventing oxidative damage.

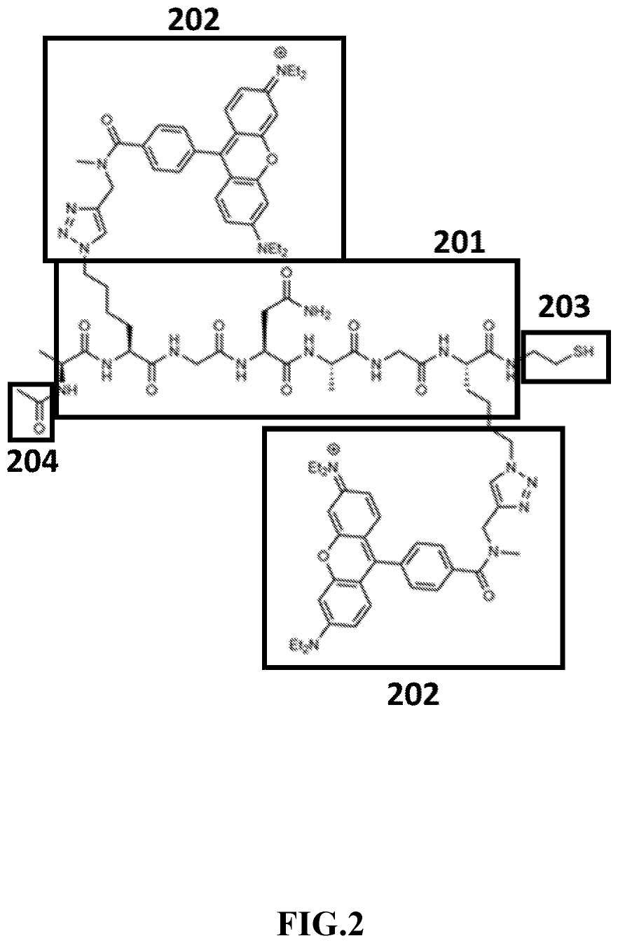

FIG. 2 provides an example of a label consistent with the present disclosure. The label comprises peptide backbone unit 201 coupled to tetramethyl rhodamine fluorophores 202 and a thiol 203 serving as a chemical handle. The tetramethyl rhodamine fluorophores are coupled to butylamine groups of lysine residues of the peptide backbone unit. The thiol chemical handle is coupled to the C-terminus of the peptide backbone unit through an amide linkage. The N-terminus of the peptide backbone unit is chemically blocked with a blocking group (e.g., an acyl group) 204, thereby diminishing the reactivity of the label.

In some cases, the blocking group can block the N-terminus from degradation (e.g., Edman degradation). In some cases, the chemical handle (e.g., thiol chemical handle) can be synthesized using a polymer from a chlorotrityl resin. In other cases, the chemical handle can be synthesized from other resins that results in a functional handle. One or more polymer backbone blocking groups can conjugate to peptides through one terminus without comprising other functionalities.

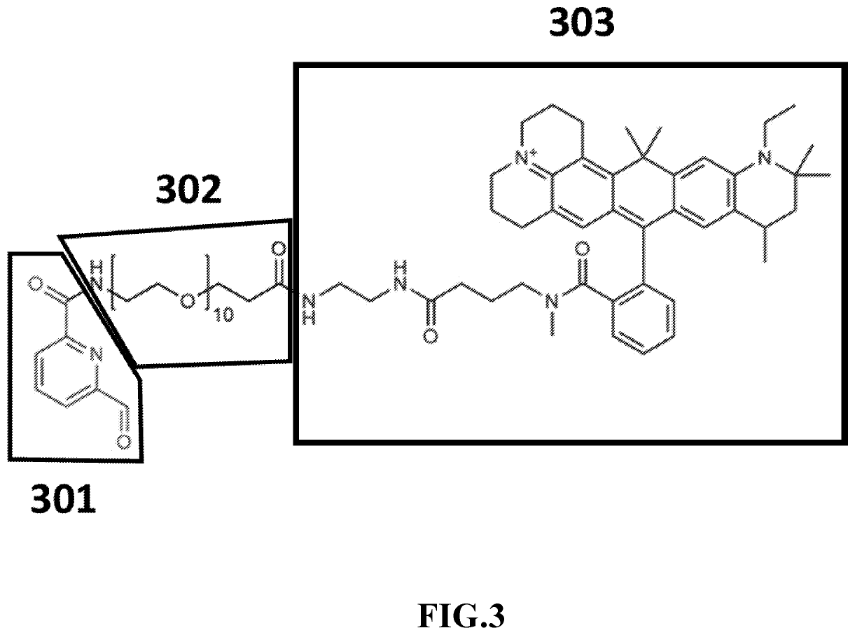

FIG. 3 provides a further example of a label consistent with the present disclosure. This example covers a label comprising a non-peptidic backbone unit. The label comprises an oligoethylene glycol backbone 301 coupled to a pyridinecarboxaldehyde chemical handle 302 and an ATTO647N dye 303.

### Backbone Units

A backbone unit may separate a chemical handle and a detectable moiety, thereby positioning the detectable moiety away from any target species to which the label couples. The backbone unit may orient the detectable moiety to diminish its interactions with the detectable moieties of other labels. The backbone unit may also be configured to repel other labels. For example, a backbone unit may comprise a plurality of phenylalanine residues which sterically repel other proximally coupled labels.

In some cases, the backbone unit comprises a stable (e.g., a rigid or a semi-rigid) conformation which constrains the location and/or orientation of the detectable moiety relative to the chemical handle. The backbone unit may comprise a locked bond or structure, such as a double or multicyclic structure, which fixes an aspect of its geometry, for example by preventing rotation. The backbone unit may comprise intramolecular interactions, such as dipole-dipole, charge-dipole, charge-charge, van der Waals, or hydrophobic packing which impart stability to a particular conformation. For example, a backbone unit may comprise a peptide configured to adopt a specific secondary structure, such as an alpha-helix or a beta-sheet that constrains the position of the detectable moiety relative to the chemical handle. In some cases, the conformation is stable at 25° C. (e.g., in aqueous buffer or in a water-DMSO mixture). In some cases, the conformation is stable at 30° C. In some cases, the conformation is stable at 40° C. In some cases, the conformation is stable at 50° C. In some cases, the conformation is stable at 60° C. In some cases, the conformation is stable at 75° C. In some cases, the conformation is stable at 90° C. In some cases, the conformation is stable at various pH, root-mean-square distributions, and/or salt percentages.

A backbone unit conformation may also limit the relative orientation of the detectable moiety. In some cases, the backbone unit comprises steric, dipole, or charged interactions with the detectable moiety which fix or inhibit its orientation. In some cases, the detectable moiety is coupled to the backbone unit in a manner which restricts its reorientation. For example, the detectable moiety may be coupled to the backbone unit by a double bond. In some cases, the backbone unit provides charge or steric interactions which limit the orientational mobility of the detectable moiety. In some cases, an orientation of a detectable moiety of a labeling reagent comprises an average angular deviation of at most 120 degrees (e.g., relative to a chemical handle of the labeling reagent). In some cases, an orientation of a detectable moiety of a labeling reagent comprises an average angular deviation of at most 90 degrees. In some cases, an orientation of a detectable moiety of a labeling reagent comprises an average angular deviation of at most about 60 degrees. In some cases, an orientation of a detectable moiety of a labeling reagent comprises an average angular deviation of at most about 45 degrees. In some cases, an orientation of a detectable moiety of a labeling reagent comprises an average angular deviation of at most about 30 degrees. In some cases, an orientation of a detectable moiety of a labeling reagent comprises an average angular deviation of at most 15 degrees. For example, a label may orient a fluorescent detectable moiety relative to a chemical handle such that the detectable moiety exhibits negligible quenching with detectable moieties of other nearby labels.

The backbone unit may distance the detectable moiety from a chemical handle (and thereby a target species to which the label couples), thereby diminishing the likelihood for interactions with a detectable moiety of another label. A detectable moiety may comprise an average distance of at least 5 Å, at least 8 Å, at least 10 Å, at least 12 Å, at least 15 Å, at least 20 Å, at least 25 Å, at least 30 Å, at least 40 Å, or at least 50 Å from a chemical handle. A detectable moiety may comprise an average distance of at most 50 Å, at most 40 Å, at most 30 Å, at most 25 Å, at most 20 Å, at most 15 Å, at most 12 Å, at most 10 Å, at most 8 Å, or at most 5 Å from a chemical handle. A backbone unit may also constrain the position of a detectable moiety. Accordingly, a position of a detectable moiety may comprise an average deviation of at most about 10 nm, at most about 8 nm, at most about 6 nm, at most about 5 nm, at most about 4 nm, at most about 3 nm, or at most about 2 nm relative to a chemical handle of a detectable labeling reagent to which it is coupled.

In some cases, the backbone unit comprises a plurality of stable conformations. The backbone unit may be configured to interconvert between a first conformation and the second conformation. Conversion between the first and second conformations may be condition (e.g., temperature, osmolarity, pH, viscosity, solvent-type) dependent. For example, a backbone unit may comprise a first conformation in acidic conditions and a second conformation in alkaline conditions. Conversion between the first and second conformations may be light-mediated. For example, a backbone unit may comprise a photoswitchable domain configured to convert from a first conformation to a second conformation upon irradiation by light of a first wavelength, and to convert from the second conformation to the first conformation upon irradiation by light of a second wavelength. A backbone unit may comprise a reactive group, such as a disulfide bond, which may be chemically interconverted between two or more states to affect a conformational change by the backbone unit. A signal of a detectable moiety may comprise a dependence on backbone unit conformation. In some cases, the signal intensity, wavelength, duration, or delay (e.g., time between excitation and a generation of a phosphorescent signal) may change vary with backbone unit conformation. For example, a wavelength of a signal of a detectable moiety may change when a backbone unit to which the detectable moiety is attached converts between a first stable conformation and a second stable conformation. In some cases, the backbone unit comprises one or more flexible conformations. For example, the position or orientation of a detectable moiety relative to another detectable moiety may be flexible or may not be stable.

A backbone unit may comprise a peptide. In some cases, the peptide comprises between 4 and 20 amino acids. In some cases, the peptide comprises between 6 and 15 amino acids. In some cases, the peptide comprises between 8 and 12 amino acids. In some cases, the peptide comprises at least 4 amino acids. In some cases, the peptide comprises at least 6 amino acids. In some cases, the peptide comprises at least 8 amino acids. In some cases, the peptide comprises at least 10 amino acids. In some cases, the peptide comprises at least 12 amino acids. In some cases, the peptide comprises at least 15 amino acids. In some cases, at least a subset of the amino acids are contiguous amino acids. In some cases, the amino acids are contiguous amino acids. In some cases, the peptide comprises a cleavage site. In some cases, the cleavage site is a protease cleavage site. In some cases, the cleavage site is configured for cleavage by a chemical agent, such as cyanogen bromide. In some cases, the peptide comprises a disulfide bond. In some cases, the peptide comprises a chemical crosslink (for example a formalin crosslink).

In some cases, the peptide comprises at least 2 unique identities of amino acids. In some cases, the peptide comprises at least 3 unique identities of amino acids. In some cases, the peptide comprises at least 4 unique identities of amino acids. In some cases, the peptide comprises at least 5 unique identities of amino acids. In some cases, the peptide comprises at least 6 unique identities of amino acids. In some cases, the peptide comprises at least 8 unique identities of amino acids. In some cases, the peptide comprises at least 10 unique identities of amino acids. In some cases, the peptide comprises at least 12 unique identities of amino acids. In some cases, the amino acids are natural amino acids. In some cases, the amino acids are proteinogenic amino acids. In some cases, the amino acids comprise a post-translationally modified amino acid. In some cases, the amino acids comprise a non-natural amino acid. In some cases, the peptide comprises a non-amino acid moiety, such as 4-aminotetrolic acid. In some cases, the non-amino acid moiety is disposed within a backbone of the peptide. In some cases, the backbone unit comprises a peptide and a non-peptide moiety.

### Chemical Handles

The present disclosure provides a range of chemical handles with varying degrees of chemical and site specificity. A chemical handle may be configured to couple to a specific target, such as a nucleotide type, a specific sequence (e.g., a peptide or nucleic acid sequence), or a chemical structure (e.g., biotin). A chemical handle may comprise specificity for a location on a target, such as a peptide N- or C-terminus or a residue within an enzyme active site. A chemical handle may also comprise a degree of binding non-specificity. For example, a chemical handle may be configured to couple to multiple types of amino acids (e.g., any amino acid with a nucleophilic side chain). A chemical handle may be inert towards a backbone unit, a detectable moiety, another chemical handle (e.g., of a detectable label to which it is coupled or to another detectable label), or any combination thereof. A chemical handle may be positionally or conformationally constrained from reacting with a backbone unit, a detectable moiety, another chemical handle, or any combination thereof.

A chemical handle may be configured to couple an amino acid type (e.g., a type of amino acid residue, such as an N-terminal amino acid or a lysine residue of a peptide). In some cases, the chemical handle is configured to couple to a single amino acid type. In some cases, the chemical handle is configured to couple to at least two amino acid types. In some cases, the chemical handle is configured to couple to a chemically related group of amino acids, such as amino acids with aromatic side chains.

In some cases, the chemical handle is configured to couple to an amino acid selected from the group consisting of cysteine, lysine, tyrosine, histidine, aspartic acid, glutamic acid, tryptophan, arginine, methionine, or any combination thereof. In some cases, the chemical handle is configured to couple to cysteine, and the chemical moiety comprises an iodoacetamide, a thiol, a benzyl halide, an allyl halide, a selenocyanate, a maleimide, an alkyne, or any combination thereof. In some cases, the chemical handle is configured to couple to lysine, and the chemical moiety comprises a thiocyanate, an isothiocyanate, a maleimide, an aldehyde, an isatoic anhydride, an NHS ester, or any combination thereof. In some cases, the chemical handle is configured to couple to aspartic acid or glutamic acid, and the chemical moiety comprises an amine, an alcohol, a thiol, an organocuprate, or any combination thereof. In some cases, the chemical handle is configured to couple to tyrosine, and the chemical moiety comprises a diazonium compound. In some cases, the chemical handle is configured to couple to histidine, and the chemical moiety comprises an alpha-beta unsaturated carbonyl compound, an epoxide, or a combination thereof. In some cases, the chemical handle is configured to couple to arginine, and the chemical moiety comprises an NHS ester. In some cases, the chemical handle is configured to couple to methionine, and the chemical moiety comprises an oxaziridine compound. In some cases, the chemical handle is configured to couple to tryptophan, and the chemical moiety comprises a diazopropanoate ester.

In some cases, the chemical handle is configured to couple to a post-translationally modified amino acid type. In some cases, the post-translationally modified amino acid type comprises phosphorylation, glycosylation, nitrosylation, citrullination, sulfenylation, trimethylation, or any combination thereof. In some cases, chemical handle is configured to couple to phosphoserine, phosphothreonine, phosphotyrosine, or any combination thereof. For example, the chemical handle may comprise a disulfide configured to couple to olefins generated from phosphoserine and phosphothreonine elimination.

### Detectable Moieties

A label may comprise a detectable moiety. A label may comprise a plurality of detectable moieties. A detectable moiety may be optically detectable (e.g., fluorescent, phosphorescent, luminescent, or light absorbing). A detectable moiety may be electrochemically detectable (e.g., a redox active moiety with a characteristic oxidation or reduction potential). A detectable moiety may comprise a mass tag (e.g., for identification with mass spectrometry. A detectable moiety may comprise an identifiable sequence (e.g., a nucleic acid sequence). A detectable moiety may be configured to generate a detectable signal in a field effect transistor. A detectable moiety may identify a label to which it is attached. For example, a label comprising a chemical handle configured to couple to lysine residues may comprise a different detectable moiety than a label comprising a chemical handle configured to couple to tyrosine residues, thereby enabling the two labels to be distinguished.

A detectable labeling reagent may comprise a plurality of detectable moieties. The present disclosure provides a range of methods for limiting signal aberrations which often arise from closely spacing multiple detectable moieties. A label of the present disclosure can be configured to maintain signal fidelities of pluralities of detectable moieties coupled to a single label. A label of the present disclosure may comprise at least 2, at least 3, at least 4, at least 5, at least 6, at least 8, at least 10, or at least 15 detectable moieties. The detectable moieties may all be of the same type (e.g., all detectable moieties are fluorophores or redox-active species). The detectable moieties may be identical (e.g., all the same type of fluorophore). Two detectable moieties of a detectable label may comprise different signals. For example, a detectable label may comprise a green fluorophore and a red fluorophore for detection through separate microscope channels. Example fluorophores usable as detectable moieties according to the disclosure include but are not limited to fluorescent dye isothiocyanates, cyanine dyes (e.g. Cy3, Cy5), tetramethylrhodamine, Si-Rhodamine, Rhodamine B, Rhodamine B N,N′-dimethylethylenediamine, Rhodamine B sulfenyl chloride, Alexafluor555, Alexa Fluor 405, Atto647N, (5) 6-napthofluorescein, variants and derivations thereof, etc. In one embodiment, the fluorophore is selected from the group consisting of tetramethylrhodamine, Si-Rhodamine, Rhodamine B, Alexafluor555, Alexa Fluor 405, Atto647N, (5) 6-napthofluorescein, fluorophore-iodoacetamide (e.g., Atto647N-lodoacetamide); a fluorophore-succinimidyl ester (e.g., Atto647N-NHS), a fluorophore-amine (e.g., Atto6-(7N-Amine), a dithiolane-fluorophore (e.g. a custom synthesized fluorophore, an oxidized dithiolane-fluorophore, a reduce dithiolane-fluorophore), a fluorophore-Azide (e.g., Atto647N-Azide), Oregon Green (OG)-iodoacetamide, OG488-NHS, OG488-Azide, OG488-Tetrazine, OG514-NHS, Janelia Fluor (JF)-NHS, JF-FreeAcid, JF-Azide, JF-Dithiolane, Atto647N-Alkyne, Atto647N-FreeAcid, Atto425-NHS, Atto425-FreeAcid, Atto425-Amine, Atto425-Azide, Atto425-DBCO, SF554-NHS, azide-DBCO, methyltetrazine-norbornene, aldehyde-dithiolane, aryl iodide-boronic acid, or TexasRed-NHS, or a variants or derivative thereof.

A labeling reagent consistent with the present disclosure may comprise a backbone unit, a first detectable moiety, a second detectable moiety, and a functional handle for coupling to a target. A signal of the first detectable moiety may be minimally or negligibly affected by the presence of the second detectable moiety on the labeling reagent. Similarly, a signal of the second detectable moiety may be minimally or negligibly affected by the presence of the first detectable moiety on the labeling reagent. In some cases, a combined intensity of a signal from the first detectable moiety and a signal from the second detectable moiety is greater than either (i) an intensity of the signal from the first detectable moiety taken individually (e.g., in the absence of the second detectable moiety) or (ii) an intensity of the signal from the second detectable moiety taken individually (e.g., in the absence of the first detectable moiety). In some cases, a combined intensity of a signal from the first detectable moiety and a signal from the second detectable moiety is at least 1.2 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.3 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.4 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.5 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.6 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.7 times greater than either (i) an intensity of the signal from the first detectable moiety taken individually or (ii) an intensity of the signal from the second detectable moiety taken individually. In some cases, a combined intensity of a signal from the first detectable moiety and a signal from the second detectable moiety is at least 1.8 times greater than either (i) an intensity of the signal from the first detectable moiety taken individually or (ii) an intensity of the signal from the second detectable moiety taken individually. For example, the first and second detectable moieties may each comprise a fluorophore, and a fluorescence intensity of the detectable labeling reagent may be greater than a fluorescence intensity of either the first detectable moiety or the second detectable moiety taken individually.

In some cases, two or more detectable moieties of the detectable labeling reagent comprises a brightness that is at least about 0.1 times, 0.2 times, 0.3 times, 0.4 times, 0.5 times, 0.6 times, 0.7 times, 0.8 times, 0.9 times, 1 times, 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 4 times, or 5 times the brightness of a single detectable moiety. In some cases, two or more detectable moieties of the detectable labeling reagent comprises a brightness that is at most about 5 times, 4 times, 3 times, 2.9 times, 2.8 times, 2.7 times, 2.6 times, 2.5 times, 2.4 times, 2.3 times, 2.2 times, 2.1 times, 2 times, 1.9 times, 1.8 times, 1.7 times, 1.6 times, 1.5 times, 1.4 times, 1.3 times, 1.2 times, 1.1 times, 1 times, 0.9 times, 0.8 times, 0.7 times, 0.6 times, 0.5 times, 0.4 times, 0.3 times, 0.2 times, or 0.1 times the brightness of a single detectable moiety. In some cases, two or more detectable moieties of the detectable labeling reagent comprises a brightness that is from about 1.5 times to 2 times the brightness of a single detectable moiety.

### Multiple Label Systems

An aspect of the present disclosure provides a system comprising a first label within 50 Å of a second label, each label comprising (i) a chemical handle for coupling to a target species, (ii) a backbone unit, and (iii) a detectable moiety; wherein a detectable signal of the detectable moiety of the first label and a detectable signal of the detectable moiety of the second label comprise a greater combined intensity than either the detectable signal of the first label in the absence of the second label or the detectable signal of the second label in the absence of the first label. In some cases, the combined intensity is about equal to the sum of the intensity of the detectable signal of the first label in the absence of the second label and the intensity of the detectable signal of the second label in the absence of the first label. In some cases, the combined intensity is at least 60% of the sum of the intensity of the detectable signal of the first label in the absence of the second label and the intensity of the detectable signal of the second label in the absence of the first label. In some cases, the combined intensity is at least 70% of the sum of the intensity of the detectable signal of the first label in the absence of the second label and the intensity of the detectable signal of the second label in the absence of the first label. In some cases, the combined intensity is at least 80% of the sum of the intensity of the detectable signal of the first label in the absence of the second label and the intensity of the detectable signal of the second label in the absence of the first label. In some cases, the combined intensity is at least 90% of the sum of the intensity of the detectable signal of the first label in the absence of the second label and the intensity of the detectable signal of the second label in the absence of the first label. In some cases, the detectable moiety of the first label is identical to the detectable moiety of the second label. In some cases, the first label and the second label are identical. For example, a fluorescence intensity of a detectable labeling reagent comprising a first fluorophore and a second fluorophore may be greater than a fluorescence intensity of the first fluorophore in the absence of the second fluorophore and a fluorescence intensity of the second fluorophore in the absence of the first fluorophore.

Similarly, the first detectable moiety and the second detectable moiety may comprise substantially similar signals as when taken in isolation. For example, an absorbance maximum of the first detectable moiety of the detectable labeling reagent may be within 50 nm, within 40 nm, within 30 nm, within 20 nm, within 15 nm, within 10 nm, or within 5 nm of the absorbance maximum of the first detectable moiety taken individually (e.g., in the absence of the second detectable moiety). Similarly, an absorbance minimum of the first detectable moiety of the detectable labeling reagent may be within 50 nm, within 40 nm, within 30 nm, within 20 nm, within 15 nm, within 10 nm, or within 5 nm of the absorbance maximum of the first detectable moiety taken individually.

A label of the present disclosure may comprise closely spaced detectable moieties. The first detectable moiety and the second detectable moiety may be spaced by an average distance of at most 50 nm, at most 40 nm, at most 30 nm, at most 25 nm, at most 20 nm, at most 15 nm, at most 12 nm, at most 10 nm, at most 8 nm, at most 6 nm, or at most 5 nm. The first detectable moiety and the second detectable moiety may be spaced by an average distance of at least 5 nm, at least 6 nm, at least 8 nm, at least 10 nm, at least 15 nm, at least 20 nm, at least 30 nm, at least 40 nm, or at least 50 nm.

Two labels of a detectable moiety may comprise constrained relative orientations. For example, the first detectable moiety and the second detectable moiety may each be constrained from rotating by more than about 150 degrees, more than about 120 degrees, more than about 90 degrees, more than about 60 degrees, more than about 45 degrees, more than about 30 degrees, or more than about 15 degrees about an average orientation. Furthermore, the first detectable moiety may be constrained from rotating relative to the second fluorophore by more than about 150 degrees, more than about 120 degrees, more than about 90 degrees, more than about 60 degrees, more than about 45 degrees, more than about 30 degrees, or more than about 15 degrees. Accordingly, a label may constrain the first detectable moiety and the second detectable moiety in relative orientations which diminish their interactions (e.g., quenching). Such alignment can be particularly important for diminishing fluorophore-fluorophore interactions. In many cases, inter-fluorophore quenching comprises a strong dependence on dipole-alignment between fluorophores. Accordingly, two fluorophores of a label may comprise diminished or minimal dipole alignment.

### Selective Amino Acid Labeling

Various aspects of the present disclosure provide methods for selectively labeling types (e.g., lysine, tyrosine, or phosphotyrosine) or groups (e.g., carboxylate side chain-containing or aromatic side chain-containing) of amino acids. A composition, system, or method of the present disclosure may selectively label cysteine, lysine, tyrosine, histidine, glutamic acid, aspartic acid, tyrosine, threonine, serine, arginine, N-terminal amines, C-terminal carboxyl-groups, or any combination thereof. A composition, system, or method may selectively label a group of amino acids, for example, a substituted maleimide reagent may couple to lysine and cysteine residues present in a sample.

The present disclosure provides a range of reagents for selectively labeling specific amino acid types (e.g., cysteine) and groups of amino acids (e.g., carboxylate side chain-containing amino acids, such as glutamate and aspartate). Non-limiting examples of cysteine-specific labels may include certain iodoacetamides, thiols, benzyl and allyl halides, selenocyanates, maleimides, and alkynes (e.g., certain alkynoic amides). In some cases, a maleimide may be configured to couple to cysteine and lysine. An example of a cysteine labeling scheme, in which a cysteine thiol nucleophilically couples to an iodoacetamide, is outlined in Scheme 1 below.

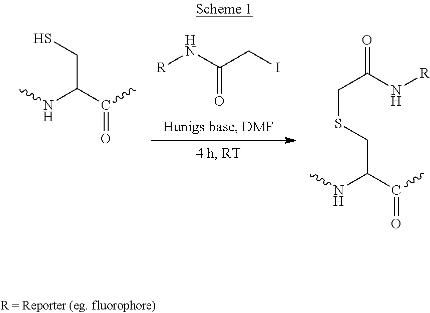

Non-limiting examples of lysine-specific labels may include certain thiocyanates and isothiocyanates, maleimides, aldehydes, isatoic anhydrides, and NHS esters. For example, a lysyl butylamine sidechain may be selectively coupled to an NHS ester, as outlined in Scheme 2.

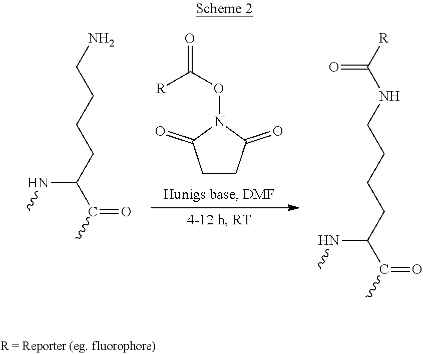

Peptide carboxylates (e.g., glutamate, aspartate, and C-terminal carboxylates) may be labeled through nucleophilic coupling operations. An example of such a coupling process is provided in Scheme 3, which illustrates carboxyl conversion to amide conversion via amine-based nucleophilic substitution.

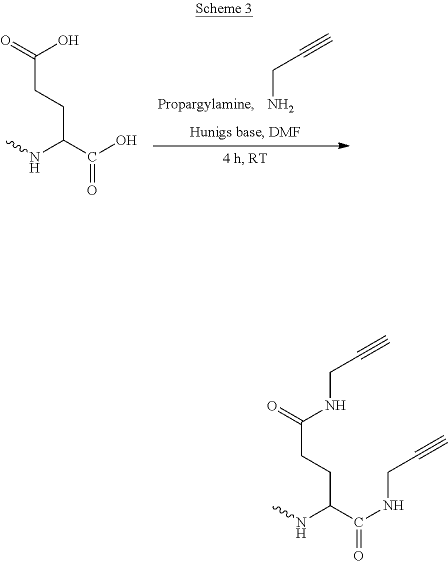

Scheme 4 provides an example of tyrosine-specific labeling. The position adjacent (e.g. ortho to) the tyrosine phenol hydroxyl carbon can be labeled through a two-step labeling process using a bifunctional diazonium reagent. Following diazo-coupling to tyrosine, a second reagent (such as a dithiolane) may optionally be coupled to the diazo label (e.g., to selectively couple a detectable moiety to the labeled tyrosine). Alternatively, the diazonium reagent may comprise a detectable moiety or may lack chemically reactive handles for further coupling.

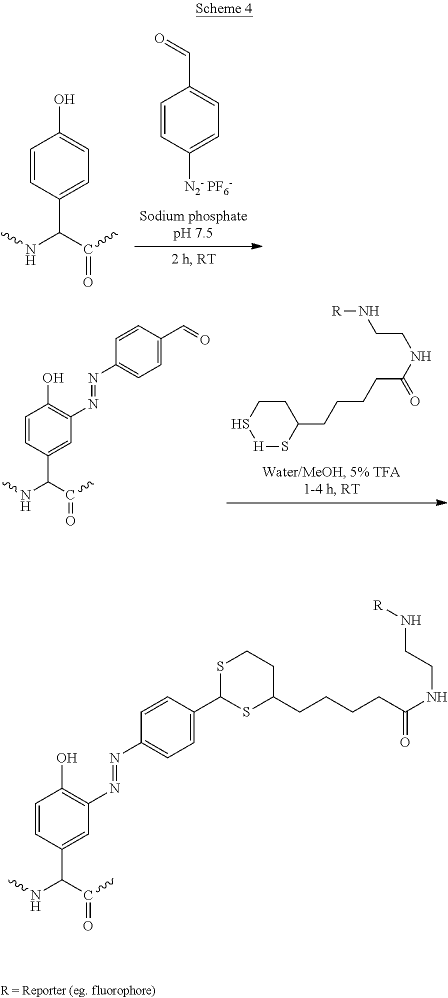

Scheme 5 provides an example of a histidine coupling scheme. A histidine imidazole nitrogen can be labeled through a two-step labeling process using an alpha-beta unsaturated carbonyl compound, such as 2-cyclohexenone. The alpha-beta unsaturated carbonyl compound may react with histidine in a nucleophilic addition reaction. The alpha-beta unsaturated carbonyl may comprise a detectable moiety. Following histidine coupling, the alpha-beta unsaturated carbonyl may be further coupled to an additional label, such as a dithiolane. Histidine may alternatively be selectively coupled to an epoxide reagent.

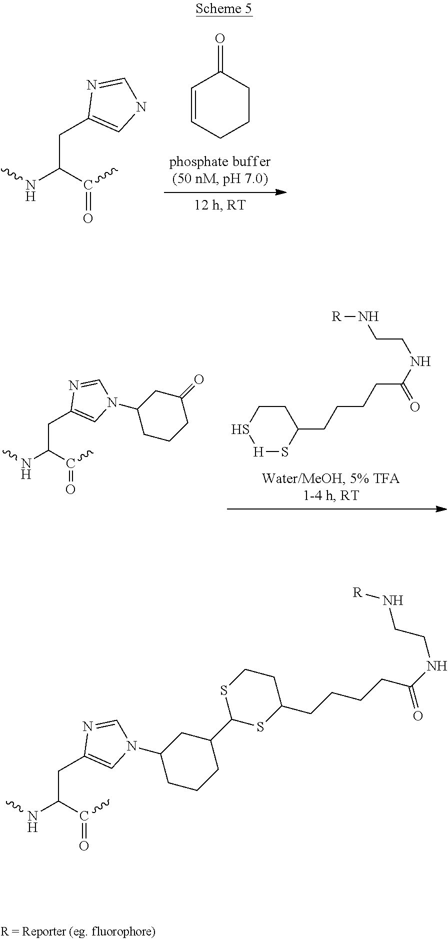

Scheme 6 provides an example of an arginine labeling mechanism. An arginine guanidinium can be acylated (e.g., labeled with an NHS ester with the aid of Barton's base). This example reaction may show cross-reactivity or interference by primary amines (e.g., N-terminus, lysine) or thiols (e.g., cysteine), and thus may be performed after N-terminal support immobilization and cysteine and lysine labeling in order to prevent or diminish cross-reactivity.

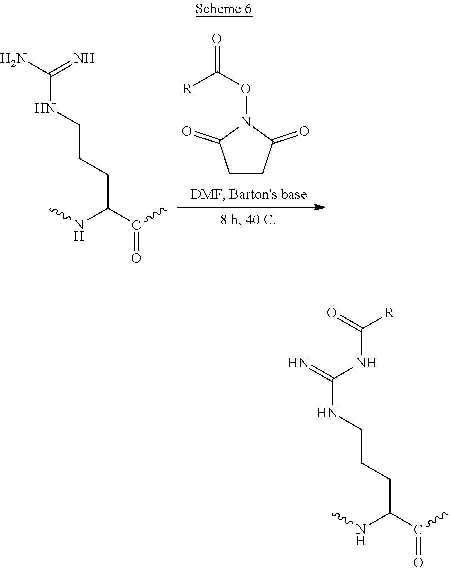

Methionine comprises a relatively low nucleophilicity and can often be selectively labeled by a redox based scheme where an oxaziridine group reacts specifically with a methionine thioether without cross-reacting with cysteine (Scheme 7). The bond formed is stable to reducing agents such as TCEP.

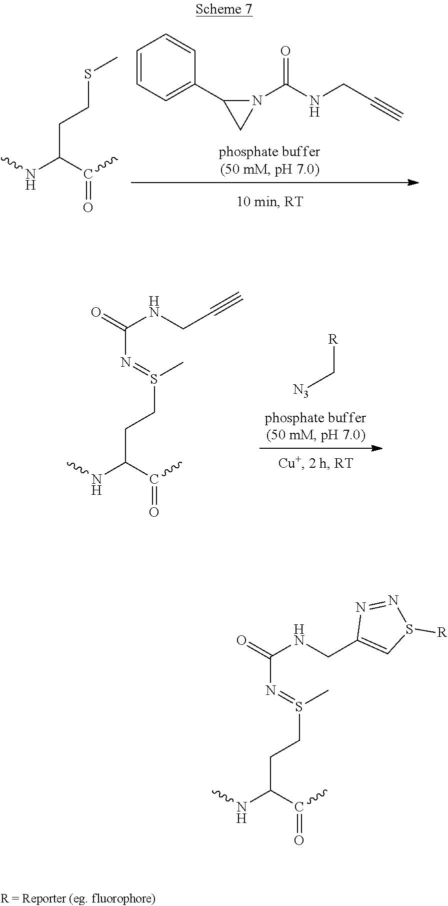

Scheme 8 provides an example of a tryptophan labeling scheme. A tryptophan indole may couple to a diazopropanoate ester, yielding a tertiary amine derivatized tryptophan, The coupling may be metal-catalyst mediated, for example by a dirhodamine (II) tetraacetate complex, which may enhance the selectivity for tryptophan over other amino acid types.

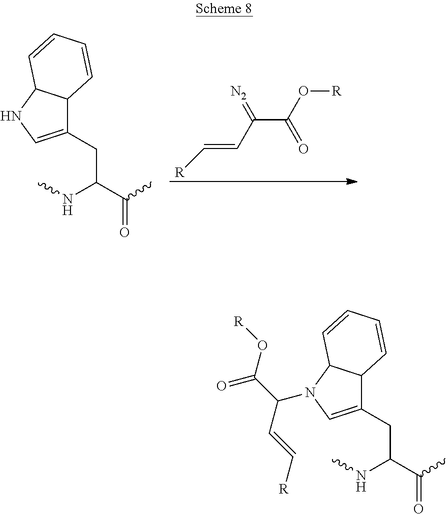

Phosphorylated amino acids such as phosphoserine, phosphotyrosine, or phosphothreonine can be selectively labeled. Such a labeling method may distinguish between types of phosphorylated amino acids. For example, Scheme 9 below provides a phosphoryl beta-elimination followed by a label conjugate addition (e.g., a Michael acceptor reaction) step for selectively labeling of phosphoserine (pSer) and phosphothreonine (pThr) over other phosphorylated amino acids such as phosphotyrosine (pTyr). A subsequent pan-phospho labeling method can be implemented to label pTyr.

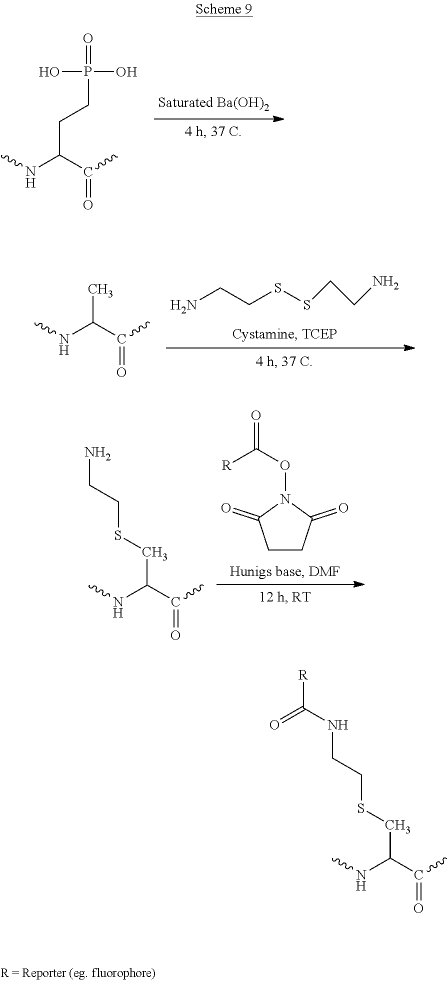

### Peptide Degradation

The present disclosure provides a range of chemical and enzymatic techniques for mild and sequential protein degradation. Degradation can be utilized in a range of peptide sequencing and analysis methods, for example to determine the order or identity of particular amino acids in a fluorosequencing assay. A peptide or protein may be iteratively subjected to cleavage conditions to determine the sequence of at least a portion of its sequence. The entire sequence of a peptide may be determined using the methods and compositions described herein. Controlled amino acid removal (e.g., N- or C-terminal amino acid removal) may be carried out through a variety of techniques including, for example, Edman degradation, organophosphate degradation, or proteolytic cleavage. In some instances, Edman degradation is used to remove a single terminal amino acid residue from a peptide N- or C-terminus. In some instances, the N-terminal amino acid residue is selectively removed from a peptide. A chemical or enzymatic technique for removing a terminal amino acid may remove a defined number of (e.g., exactly one, exactly two, at most two) amino acids. Accordingly, a method for analyzing a peptide may comprise successive degradation and analysis operations, such that the removal of a defined number of amino acids from an N-terminus or C-terminus per operation provides position and sequence specific amino acid identifications during analysis. A chemical or enzymatic technique for removing a terminal amino acid may cleave a peptide at a defined location (e.g., only in between two alanine residues, or only at the peptide bond connecting an N-terminal amino acid to the remainder of a peptide).

An Edman degradation method may comprise chemically functionalizing a peptide N-terminus or C-terminus (e.g., to form a thiourea or a guanidinium derivative of an N-terminal amine), and then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A diactivated phosphate or phosphonate may be used for peptide cleavage. Such a method may utilize an acid to remove a functionalized amino acid. The diactivated phosphate or phosphonate may be a dihalophosphate ester. In other embodiments, the techniques involve using an enzyme to remove the terminal amino acid residue, such as, for example, an exopeptidase or an Edmanase. For example, a method may comprise derivatizing an N-terminal amino acid of a peptide with a diactivated phosphate, and contacting the peptide with an Edmanase with cleavage activity toward phosphate-functionalized N-terminal amino acids.

A cleavage method (e.g., a cleavage method implemented within a sequencing method) may comprise enzymatic cleavage. The cleavage method may comprise the use of a single protease, a series of proteases (e.g., provided in a specific order), or a combination of proteases. Proteases and their associated cleavage sites are provided in TABLE 1. A cleavage method may comprise decoupling a peptide barcode from a molecule. For example, a peptide barcode may comprise a cleavable linker comprising a cleavage site recognized by a protease listed in TABLE 1. In such cases, the sequence of the cleavage site may be present in the cleavable linker and absent in the peptide barcode. A cleavage method may comprise fragmenting a peptide barcode (e.g., cleaving an internal peptide bond prior to peptide barcode sequencing).

TABLE 1
Proteases

| Protease | Cleavage Site |
|---|---|
| Carboxypeptidase A | C-terminal exopeptidase |
| Carboxypeptidase B | C-terminal exopeptidase; specific for lysine or arginine |
| Carboxypeptidase P | C-terminal exopeptidase |
| Carboxypeptidase Y | C-terminal exopeptidase |
| Cathepsin C | N-terminal exopeptidase; removes N-terminal dipeptide (except |
| | when N-terminal amino acid is lysine or arginine, or when 2^{nd} or |
| | 3^{rd} amino acid from N-terminal is proline) |
| Chymotrypsin | C-terminal exopeptidase; specific for phenylalanine, |
| | tryptophan, and tyrosine |
| Clostripain | arginine |
| Elastase | Alanine, Valine, Serine, Glycine, Leucine, or Isoleucine |
| Endoproteinase Arg-C | Arginine |
| Endoproteinase Glu-C | Glutamic Acid |
| Endoproteinase Lys-C | Lysine |
| Glutamyl endopeptidase | Glutamic acid |
| Kallikrein (Plasma) | Lysine or Arginine |
| Papain | Lysine or Arginine followed by a hydrophobic residue |
| Pepsin | Leucine, Phenylalanine, Tryptophan or Tyrosine |
| Proteinase K | Aliphatic and aromatic amino acids |
| Subtilisin | Hydrophobic amino acids |
| TEV Protease | Specific for the sequences Glutamic acid-Asparagine-Leucine- |
| | Tyrosine-Phenylalanine-Glutamine-Glycine and Glutamic acid- |
| | Asparagine-Leucine-Tyrosine-Phenylalanine-Glutamine-Serine; |
| | cleaves between Glutamine-Glycine or Glutamine-Serine |
| Thermolysin | Isoleucine, Methionine, Phenylalanine, Tryptophan, Tyrosine, |
| | or Valine |
| Trypsin | Lysine or Arginine |

Peptide cleavage may comprise chemical cleavage. Examples of chemical cleavage reagents consistent with the present disclosure include cyanogen bromide, BNPS-skatole, formic acid, hydroxylamine, and 2-nitro-5-thiocyanobenzoic acid. A peptide barcode May comprise a chemically cleavable moiety, such as a disulfide. A peptide barcode may be coupled to a molecule by a linker which comprises a chemically cleavable moiety. A peptide barcode may be coupled to a molecule by a chemically cleavable bond. A cleavage method may comprise a combination (e.g., parallel or sequential use) of chemical and enzymatic cleavage reagents. A cleavage method may comprise activating (e.g., functionalizing) an amino acid for chemical or enzymatic cleavage. For example, a method may comprise derivatizing an N-terminal amino acid residue of a peptide, and then contacting the peptide with an ‘Edmanase’ enzyme configured to remove the derivatized N-terminal amino acid residue.

Peptide cleavage conditions may be achieved with a solvent. The solvent may be an aqueous solvent, an organic solvent, or a combination or mixture thereof. The solvent may be an organic solvent. The organic solvent may comprise a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and a C₁-C₁₂ haloalkane. The mixture of two or more solvents may be a mixture of DMF and DCM. The mixture of solvents may be any combination thereof.

A degradation process may comprise a plurality of operations. For example, a method may comprise an initial operation for derivatizing a terminal amino acid of a peptide, and a subsequent operation for cleaving the derivatized terminal amino acid from the peptide. One such method comprises organophosphorus compound-mediated N-terminal functionalization and removal, and thus provides an alternative to the isothiocyanate (e.g., phenyl isothiocyanate) based processes of some Edman degradation schemes.

An organophosphate-based degradation scheme may comprise dissolving a peptide in an organic solvent or organic solvent mixture (e.g., a mixture of dichloromethane and dimethylformamide) in the presence of an organic base (e.g., triethylamine, N, N-diisopropylethylamine (DIPEA), 1,8-diazabicyclo[5.4.0]undec-7-ene (DBU), pyridine, 1,5-diazabicyclo(4.3.0)non-5-ene, 2,6-di-tert-butylpyridine, imidazole, histidine, sodium carbonate, etc.). The peptide may then be contacted with at least one organophosphorus compound. The cleavage of the peptide or protein N-terminus may be initiated through the addition of a weak acid (e.g., formic acid in water). The cleavage of the peptide or protein N-terminus may also be initiated with water. The resulting products may include the terminal amino acid of the peptide or protein released from the peptide as a phosphoramide and the peptide or protein that is shortened by the terminal amino acid residue, which comprises a free N-terminus that can be used to perform a subsequent cleavage reaction.

A cleavage method may comprise digesting a peptide to generate fragments of a predetermined average length. The cleavage method may generate peptides (e.g., by acting upon a complex mixture of peptides, such as cell lysate) with an average length of at least 5 amino acids, at least 8 amino acids, at least 10 amino acids, at least 12 amino acids, at least 15 amino acids, at least 20 amino acids, at least 25 amino acids, at least 30 amino acids, at least 40 amino acids, or at least 50 amino acids. The cleavage method may generate peptides with an average length of at most 50 amino acids, at most 40 amino acids, at most 30 amino acids, at most 25 amino acids, at most 20 amino acids, at most 15 amino acids, at most 12 amino acids, at most 10 amino acids, at most 8 amino acids, or at most 5 amino acids. The cleavage method may generate peptide fragments with an average length of between 5 and 20 amino acids, between 5 and 30 amino acids, between 10 and 20 amino acids, between 10 and 30 amino acids, between 12 and 18 amino acids, between 15 and 30 amino acids, between 20 and 40 amino acids, or between 30 and 50 amino acids.

A reaction mixture may comprise a stoichiometric or an excess concentration of a cleavage compound (e.g., relative to the concentration of peptides to be cleaved). The reaction mixture may comprise at least about 0.001% v/v, about 0.01% v/v, about 0.1% v/v, about 1% v/v, about 5% v/v, about 10% v/v, about 15% v/v, about 20% v/v, about 30% v/v, about 40% v/v, about 50% v/v, or more of the cleavage compound. The reaction mixture may comprise at most about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound. The reaction mixture may comprise about 5% v/v of the cleavage compound.

The reaction may be performed at a temperature of at least about 0° C., at least about 5° C., at least about 10° C., at least about 15° C., at least about 20° C., at least about 25° C., at least about 30° C., at least about 40° C., at least about 50° C., at least about 60° C., at least about 70° C., at least about 80° C., or at least about 90° C. The reaction may be performed at a temperature of at most about 90° C., at most about 80° C., at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature. The reaction may be performed at close to 0° C. or below 0° C. (e.g., in the presence of an antifreeze).

The peptide and the cleavage compound may be mixed or incubated for at least about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, about 24 hours, or more. The peptide and the cleavage compound may be mixed or incubated for at most about 24 hours, about 20 hours, about 16 hours, about 12 hours, about 10 hours, about 8 hours, about 6 hours, about 4 hours, about 3 hours, about 2 hours, about 1 hour, about 50 minutes, about 40 minutes, about 30 minutes, about 20 minutes, about 10 minutes, about 5 minutes, about 1 minute, or less. The peptide and the cleavage compound may be mixed or incubated from about 1 minute to about 24 hours, 5 minutes to about 6 hours, 5 minutes to about 2 hours, or 5 minutes to about 30 minutes.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (peptides, nucleic acids, lipids, small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffycoat proteins may be partitioned and separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and analyzing the peptides by a fluorosequencing method of the present disclosure. A method may comprise capturing peptides from a sample using a C-terminal capture reagent, and analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and a peptides from a human plasma sample, sequencing the cell free DNA (e.g., to identify a cancer marker), and performing proteomic analysis on the plasma proteins.

### Computer Systems

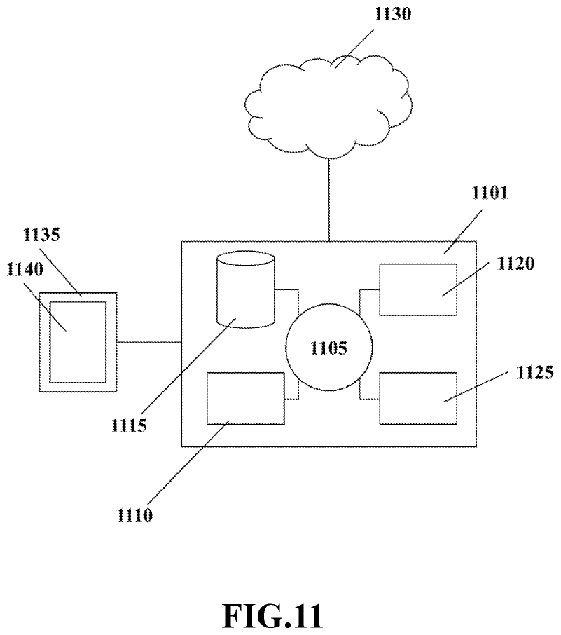

The present disclosure provides computer systems that are programmed to implement methods of the disclosure. FIG. 11 shows a computer system 1101 that is programmed or otherwise configured to implement methods or parts of methods disclosed herein, including compiling, analyzing, and displaying data obtained through the present methods. The computer system 1101 may regulate various aspects of the present disclosure, such as, for example, controlling cell partitioning and optical imaging devices. The computer system 1101 may be an electronic device of a user or a computer system that is remotely located with respect to the electronic device. The electronic device may be a mobile electronic device.

The computer system 1101 includes a central processing unit (CPU, also “processor” and “computer processor” herein) 1105, which may be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 may be a data storage unit (or data repository) for storing data. The computer system 1101 may be operatively coupled to a computer network (“network”) 1130 with the aid of the communication interface 1120. The network 130 may be the Internet, an internet and/or extranet, or an intranet and/or extranet that is in communication with the Internet. The network 1130 in some cases is a telecommunication and/or data network. The network 1130 may include one or more computer servers, which may enable distributed computing, such as cloud computing. The network 1130, in some cases with the aid of the computer system 1101, may implement a peer-to-peer network, which may enable devices coupled to the computer system 1101 to behave as a client or a server.

The CPU 1105 may execute a sequence of machine-readable instructions, which may be embodied in a program or software. The instructions may be stored in a memory location, such as the memory 1110. The instructions may be directed to the CPU 105, which may subsequently program or otherwise configure the CPU 1105 to implement methods of the present disclosure. Examples of operations performed by the CPU 1105 may include fetch, decode, execute, and writeback.

The CPU 1105 may be part of a circuit, such as an integrated circuit. One or more other components of the system 1101 may be included in the circuit. In some cases, the circuit is an application specific integrated circuit (ASIC).

The storage unit 1115 may store files, such as drivers, libraries and saved programs. The storage unit 1115 may store user data, e.g., user preferences and user programs. The computer system 1101 in some cases may include one or more additional data storage units that are external to the computer system 1101, such as located on a remote server that is in communication with the computer system 1101 through an intranet or the Internet.

The computer system 1101 may communicate with one or more remote computer systems through the network 1130. For instance, the computer system 1101 may communicate with a remote computer system of a user (e.g., a fluorimeter or a cell sorting device). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user may access the computer system 1101 via the network 1130.

Methods as described herein may be implemented by way of machine (e.g., computer processor) executable code stored on an electronic storage location of the computer system 1101, such as, for example, on the memory 1110 or electronic storage unit 1115. The machine executable or machine readable code may be provided in the form of software. During use, the code may be executed by the processor 1105. In some cases, the code may be retrieved from the storage unit 1115 and stored on the memory 1110 for ready access by the processor 1105. In some situations, the electronic storage unit 1115 may be precluded, and machine-executable instructions are stored on memory 1110.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 1101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 may include or be in communication with an electronic display 1135 that comprises a user interface (UI) 1140 for providing, for example, orders and options for controlling flow rates in a cell sorting device. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 1105. The algorithm may, for example, determine a correlation using linear and quadratic discriminant analysis (LDA and QDA), Support Vector Machine (SVM), linear discriminant analysis (LDA), quadratic discriminant analysis (QDA), Naive Bayes, Random Forest, or any other suitable method.

### EXAMPLES

### Example 1—Combined Computational and Experimental Label Design

This example covers a combined computational and experimental method for designing peptidic labels. The method includes iterative in silico and experimental testing operations for generating labels with optimized properties, such as brightness and stability, and experimental operations for confirming attributes of the computationally derived labels. The computational operations ensure that broad peptide sequence and structural spaces are queried in the design process, while the experimental operations serve to validate the computational design operations and direct the in-silico modeling towards relevant hits.

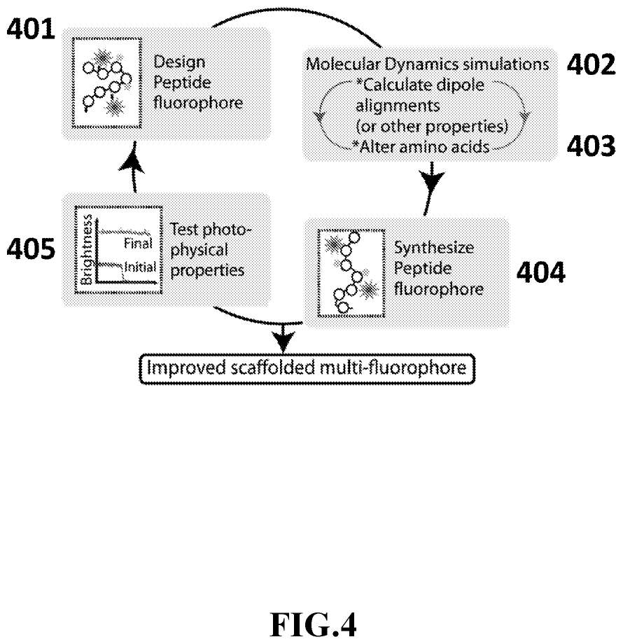

FIG. 4 provides an overview of the combined in-silico and experimental method for peptide-based label optimization. Briefly, a label design cycle begins with an input of a label structure 401. The label structure may be derived from a previous design cycle, may be generated based on a number of design parameters (e.g., peptide backbone unit length, allowable amino acid types, number of detectable moieties), or may be input manually.

Next, a series of iterative molecular dynamics (MD) simulations 402 and in silico structural variation operations 403 calculate label properties and vary label structures. The MD simulations may predict a range of label properties including stability, detectable moiety spacing and orientation, and conformational rigidity. Labels with predetermined properties (e.g., high stabilities, specific isoelectric points, high solubilities) are then varied to generate new series of alternate label structures. The in-silico structural variation operations may impart random or systematic changes to label structures. For example, the in-silico operation may impart random double amino acid substitutions on each input structure, or may generate a full library of single amino acid substitution variants. The new labels generated through in-silico structural variation may be reassessed through further MD simulations.

In a series of counterpart experimental operations, MD validated labels may be synthesized 404 and analyzed 405 for various performance metrics (e.g., brightness or stability). The highest performing labels may be computationally evaluated 402 and further varied 403. Cycles of in-silico and experimental analyses may be performed until labels with predetermined properties are generated.

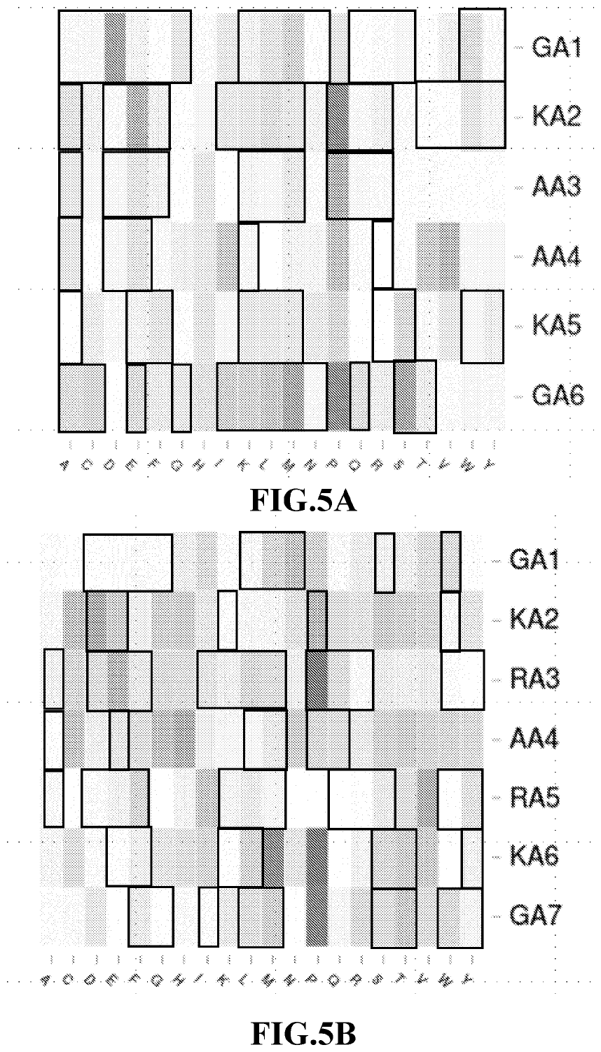

FIGS. 5A-5B provide heat maps for libraries of labels related by single amino acid substitutions. In both plots, the x-axes provide the identities of single amino acid substitutions (from left to right: alanine, cysteine, aspartic acid, glutamic acid, phenylalanine, glycine, histidine, isoleucine, lysine, methionine, asparagine, proline, glutamine, arginine, serine, threonine, valine, tryptophan, and tyrosine, respectively). The analyzed peptides comprise 6 amino acid residues with the sequence G-K-X₁-X₂-K-G (SEQ ID NO: 1). The identities of X₁ and X₂, are provided in the heat map columns. A total of nearly 400 peptides were analyzed with a peptide rotamer algorithm and Amber ff99SB force fields. The simulations were performed in 5 Å boxes with methanol as a solvent, and covered 1 picosecond of peptide conformational evolution. The heat maps of FIGS. 5A-5B provide the relative stabilities of each analyzed peptide, with gray cells enclosed in the black boxes indicating increased stabilities, grey cells not enclosed in the black boxes indicating diminished stabilities, and white cells not enclosed in the black boxes indicating unchanged stabilities relative to a reference peptide. The reference peptide has the sequence-GKAAKG (SEQ ID NO: 192).

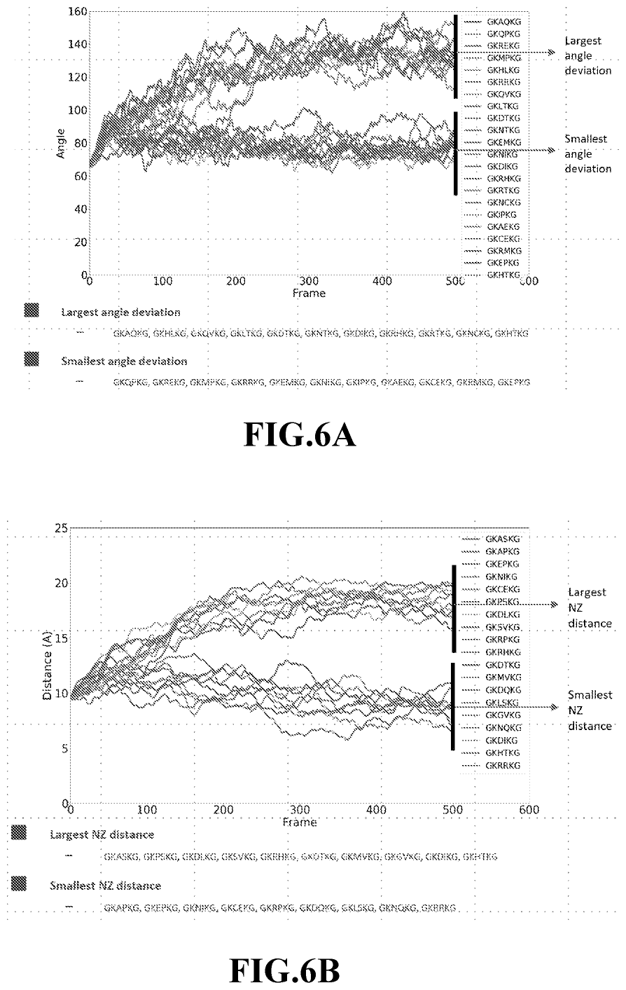

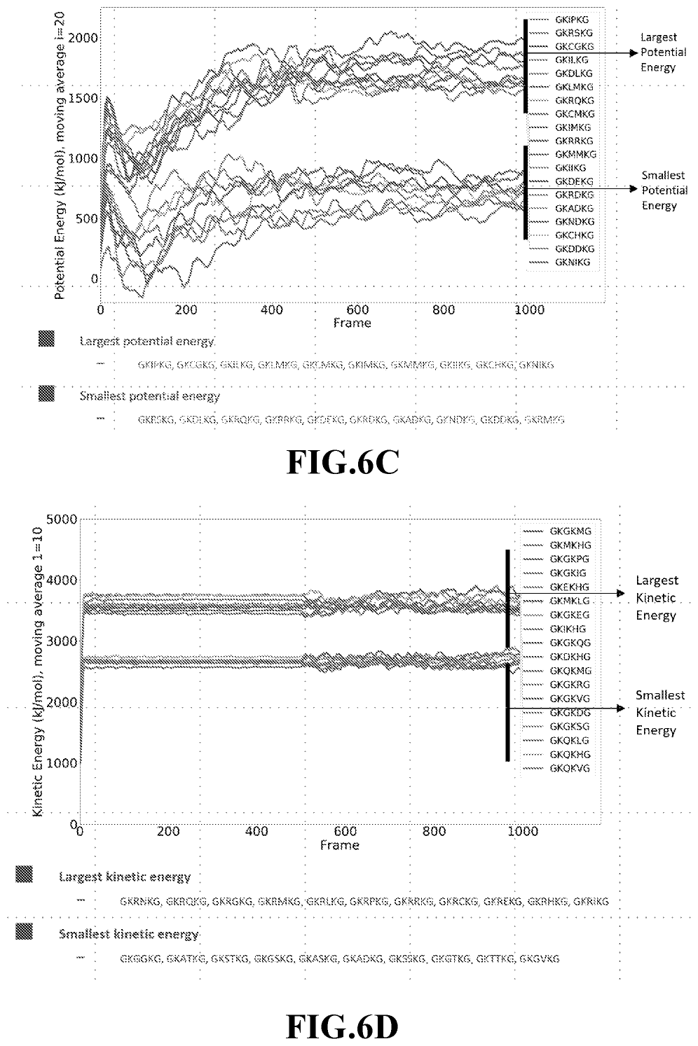

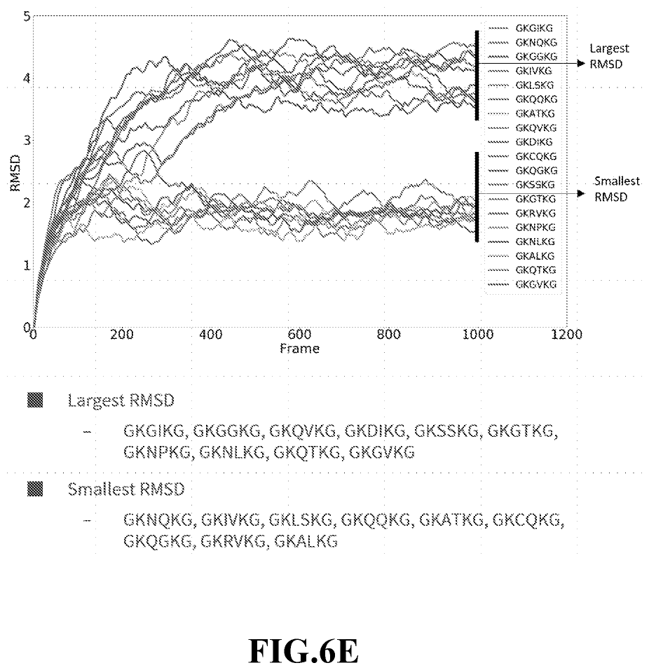

FIGS. 6A-6E summarize results for peptides with the sequence G-K-X₁-X₂-K-G (wherein X₁ and X₂ are varied between individual peptides; SEQ ID NO: 1). The x-axis for each plot provides calculation frame, with later frames corresponding to later structural optimization operations. FIGS. 6A-6B provide the angle between lysine residues and the distance between lysine side chain amines (NZ atom distance), respectively. Peptide sequences are illustrated with the most flexibility and least flexibility falling into groups with about 80 degree and about 130-degree inter-lysine backbone angles, and about 10 Å and about 18 Å NZ atom distances. Certain peptides, such as GKRRKG (SEQ ID NO: 16), exhibited small inter-lysine backbone angles and NZ atom distances, while others, such as GKDTKG (SEQ ID NO: 29), exhibited large inter-lysine backbone angles and NZ atom distances.

FIGS. 6C-6E provide potential energy, kinetic energy, and root-mean-square distributions (RMSDs) for each peptide, respectively. Potential energy (in FIG. 6C) indicates the relative stability of each peptide, with lower values indicating greater stabilities. Kinetic energy (in FIG. 6D) serves as a measure of temperature. RMSD values are relative to the starting atomic positions in each simulation, with larger values indicating a greater degree of conformational changes, and thus a greater degree of conformational flexibility. As with FIGS. 6A-B, the queried peptides exhibited bimodal distributions for potential energy, kinetic energy, and RMSD.

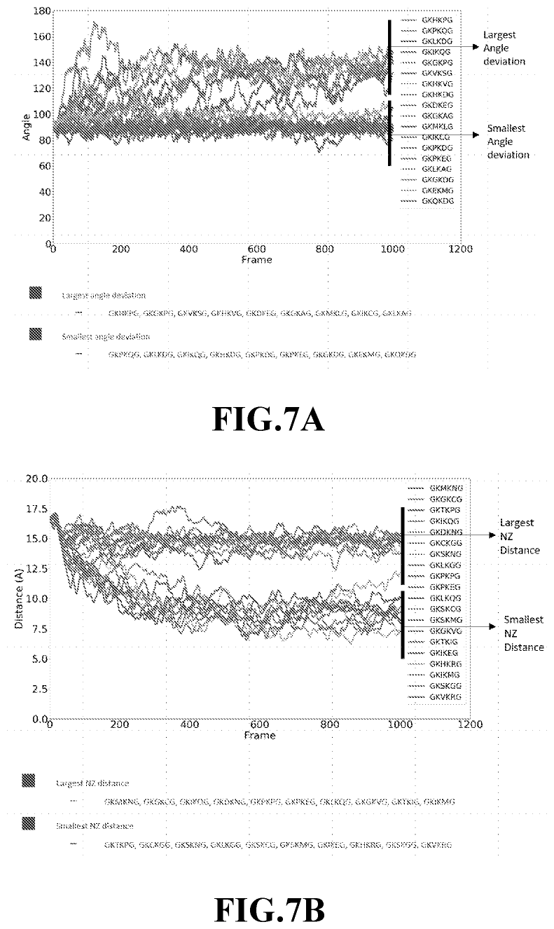

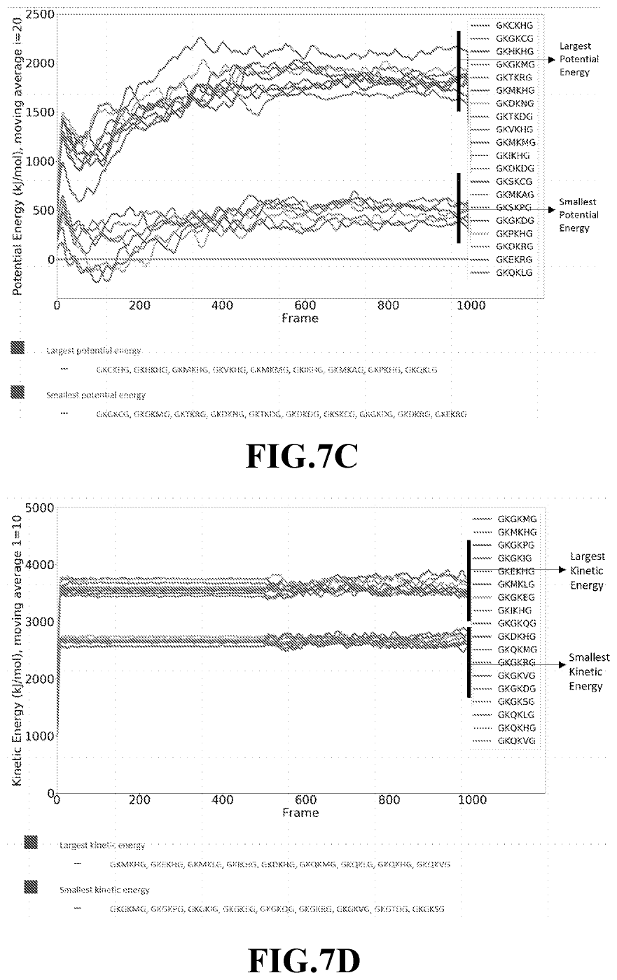

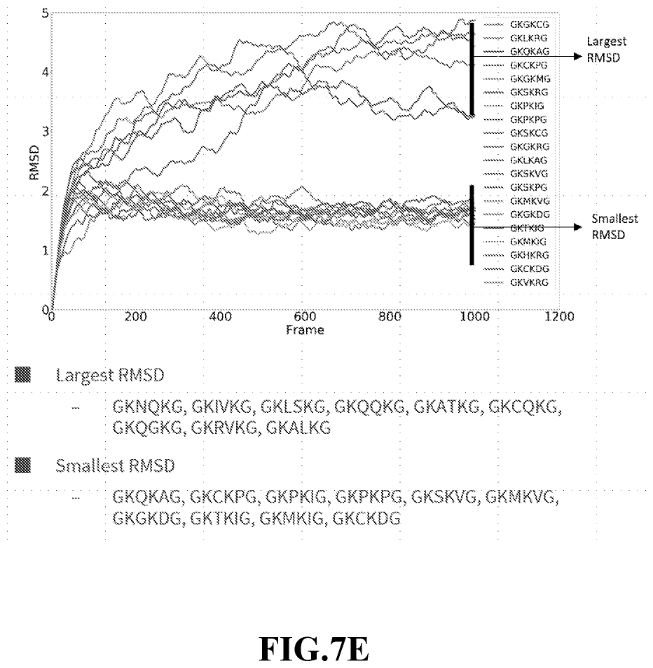

FIGS. 7A-7E summarize results for peptides with the sequence G-K-X₁-K-X₂-G (wherein X₁ and X₂ are varied between individual peptides; SEQ ID NO: 104)). The x-axis for each plot provides calculation frame, with later frames corresponding to later structural optimization operations. FIGS. 7A-7B provide inter-lysine backbone angles and NZ atom distances, respectively, for select queried peptides. FIGS. 7C-7E provide potential energy, kinetic energy, and root-mean-square distributions (RMSDs) for each peptide, respectively. As in FIG. 6, the peptides exhibit bimodal distributions for each feature, with inter-lysine backbone angles clustered around 90 degrees and 140 degrees, NZ atom distances clustered around 9 Å and 15 Å, and RMSDs clustered around 1.8 Å and 4 Å. The differences between FIGS. 6A-6E and 7A-7E indicate that changes in atom order can affect peptide properties.

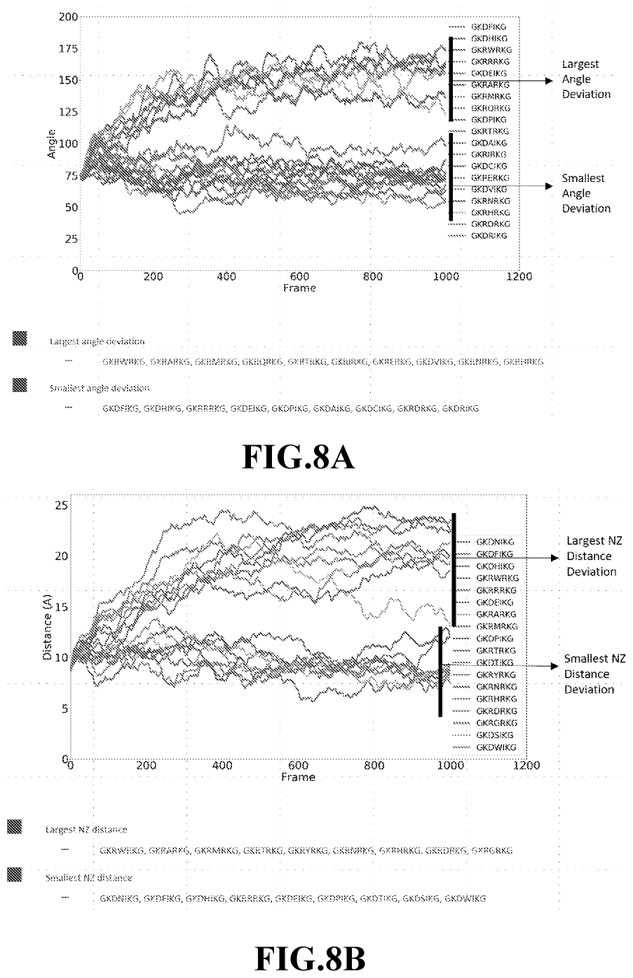

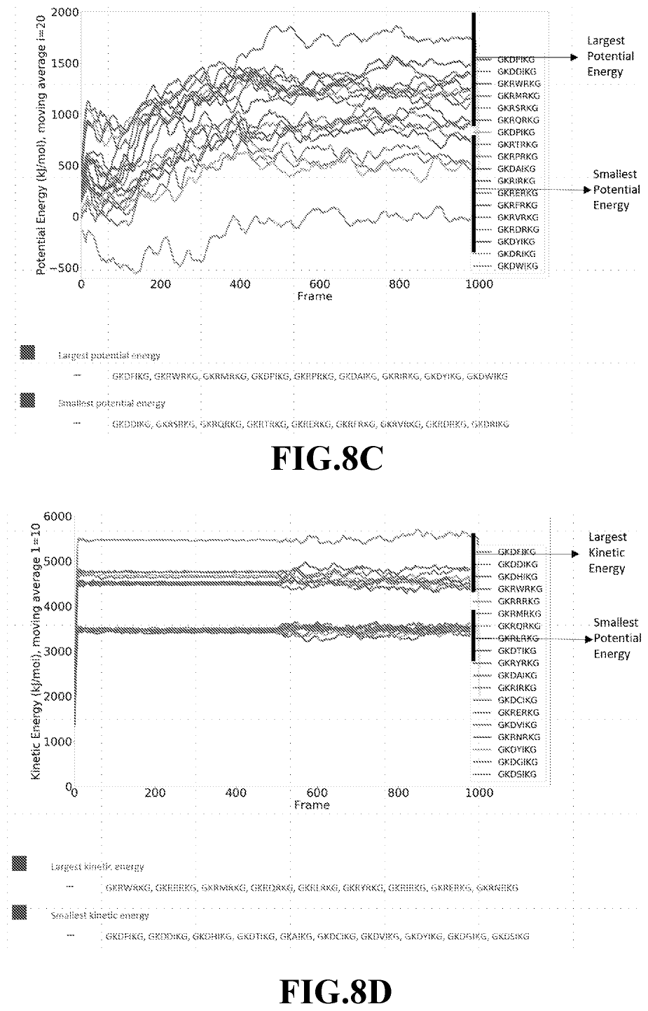

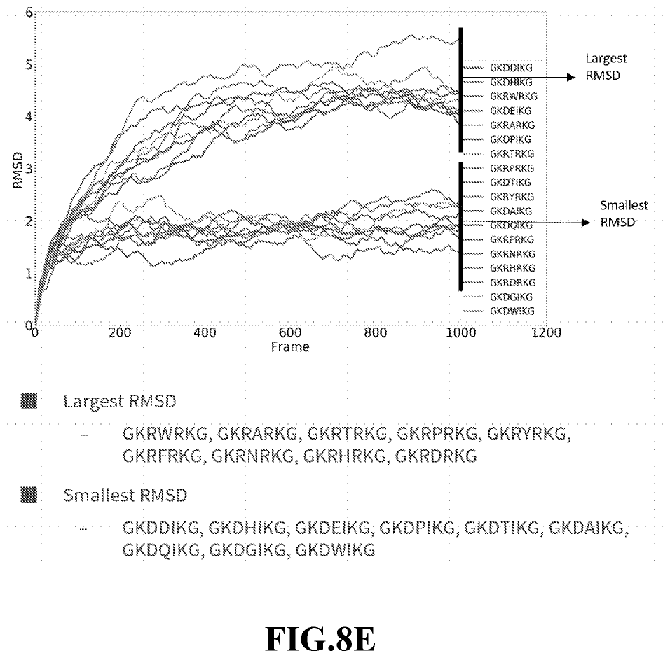

FIGS. 8A-8E summarize results for 7-mer peptides with the sequences G-K-R-X-R-K-G (SEQ ID NO: 157) and G-K-D-X-I-K-G (SEQ ID NO: 158) (wherein X is varied between individual peptides). The x-axis for each plot provides calculation frame, with later frames corresponding to later structural optimization operations. FIGS. 8A-8B provide inter-lysine backbone angles and NZ atom distances, respectively, for select queried peptides. FIGS. 8C-8E provide potential energy, kinetic energy, and root-mean-square distributions (RMSDs) for each peptide, respectively. The peptides span inter-lysine backbone angles clustered from about 50 degrees to 150 degrees, NZ atom distances from about 8 Å to about 24 Å, and RMSDs from about around 1 Å to about 5 Å.

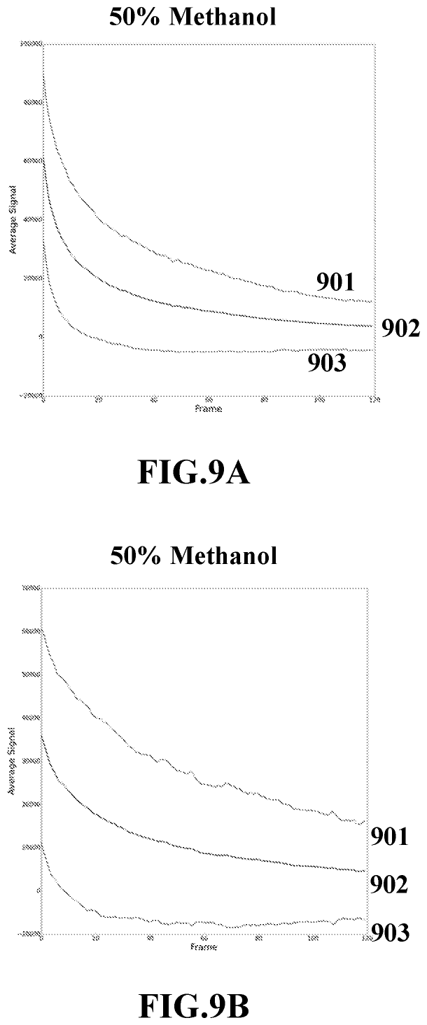

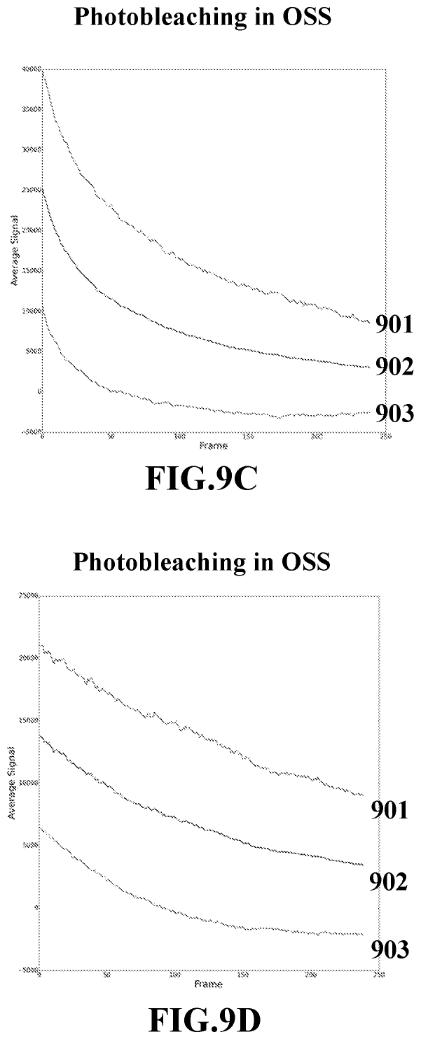

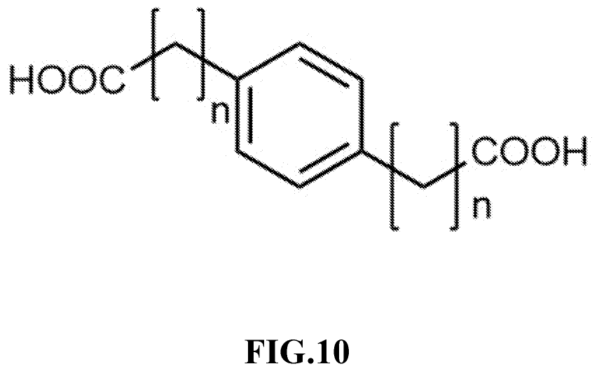

FIGS. 9A-9D provide calculated fluorophore signal intensities for select peptides, with the y-axes providing calculated signal intensity and the x-axes corresponding to optimization operation. The plot represents a photobleaching trace of a population of synthesized and rhodamine derivatized peptide molecules overlaid with its intensity—902=average signal intensity of all peptides, 901 and the 903 lines denote the bounds in the range of the peptide intensities. The peptide in the experiment are—(a) ac-AK*GNAK*-SH (SEQ ID NO: 191) and (b) ac-AK*-SH are immobilized on a Maleimide functionalized PEG-silane surface based on commonly used procedures. The imaging was done under two different conditions—50% methanol and 100% methanol with 2 mM trolox (denoted as OSS). Photobleaching was performed by irradiating a field of single peptide molecules for 120s using a 561 laser on a TIRF microscope setup. FIGS. 9A-9B represent the photo-physical behavior of the two peptides in 50% methanol and FIGS. 9C-9D indicate the photobleaching curves in OSS. The clear difference in twice brightness with two dyes is meant to serve the ability to correctly use this platform for sema-fluor development.

### EMBODIMENTS

The following embodiments are not intended to be limiting in any way.
1. A detectable labeling reagent for coupling to a target biomolecule, the detectable labeling reagent comprising:
   (i) a chemical handle configured to couple to the target biomolecule,
   (ii) a backbone unit, and
   (iii) one or more detectable moieties;
      wherein the backbone unit comprises a conformation so that, when the detectable labeling reagent is coupled to the target biomolecule, the backbone unit substantially constrains a position or an orientation of a detectable moiety of the one or more detectable moieties relative to another detectable moiety of the one or more detectable moieties that is coupled to the target biomolecule.
2. The detectable label of embodiment 1, wherein the chemical handle is configured to couple to an amino acid of the target biomolecule.
3. The detectable label of embodiment 1 or 2, wherein the detectable labeling reagent comprises two detectable moieties.
4. The detectable label any one of embodiments 1-3, wherein the chemical handle comprises a primary amine-reactive group, a primary thiol-reactive group, a thioether-reactive group, a primary alcohol-reactive group, a phenol-reactive group, a carboxylic acid-reactive group, or a hydroxyl-reactive group, or any combination thereof.
5. The detectable label of any one of embodiments 1-4, wherein the detectable labeling reagent further comprises a structure according to

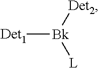

wherein Bk denotes the backbone unit;
Det₁ and Det₂ independently denote a first and a second detectable moiety; and
L denotes the chemical handle.
6. The detectable labeling reagent of any one of embodiments 1-5, wherein the backbone unit further comprises an oligopeptide structure according to:
   X₃-X₁-X₂-X₄ wherein;
   X₃ and X₄ independently denote an amino acid side chain linked to a detectable moiety; and
   X₁ and X₂ independently denote any amino acid.
7. The detectable labeling reagent of any one of embodiments 1-6, wherein the backbone unit further comprises an oligopeptide structure according to:
   X₃-X₁-X₂-X₄ wherein;
   X₃ comprises

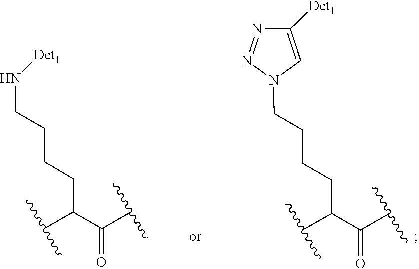

X₄ comprises

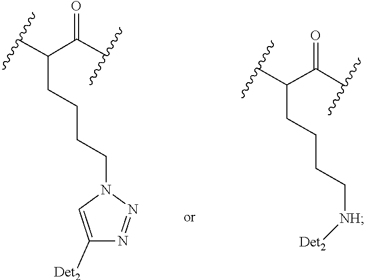

Det₁ and Det₂ independently denote first and second detectable moieties; and
X₁ and X₂ independently denote any amino acid.
8. The detectable labeling reagent according to embodiment 7, wherein the detectable label further comprises an oligopeptide structure according to:
   Ac-G-X₃-X₁-X₂-X₄-L; wherein
   Ac denotes an acetyl group;
   G denotes glycine; and
   L denotes the chemical handle.
9. The detectable labeling reagent of any one of embodiments 1-8, wherein the backbone unit substantially constrains an orientation of the first and the second detectable moiety relative to each other.
10. The detectable labeling reagent of embodiment 9, wherein the orientation of the first and the second detectable moieties comprises an average angular deviation of at most about 160 degrees relative to each other.
11. The detectable labeling reagent of embodiment 9 or 10, wherein the orientation of the first and the second detectable moieties comprises an average angular deviation of at least 60 degrees relative to each other.
12. The detectable labeling reagent of any one of embodiments 9-11, wherein the backbone unit comprises an oligopeptide according to X₃-X₁-X₂-X₄, wherein:
   X₁ is selected from the group consisting of A, Q, R, M, H, L, D, N, E, I, and C; and
   X₂ is selected from the group consisting of Q, P, E, L, R, V, T, M, I, H, and C.
13. The detectable labeling reagent of any one of embodiments 9-12, wherein the backbone unit comprises an oligopeptide according to X₃-X₁-X₂-X₄, wherein:
   X₁-X₂ is according to AQ, QP, RE, MP, HL, RR, QV, LT, DT, NT, EM, NI, DI, RH, RT, NC, IP, AE, CE, RM, EP, or HT.
14. The detectable labeling reagent of any one of embodiments 1-13, wherein the backbone unit substantially constrains a distance of the first and the second detectable moiety relative to each other.
15. The detectable labeling reagent of embodiment 14, wherein the distance of the one or more detectable moieties comprises a distance of at most about 25 angstroms.
16. The detectable labeling reagent of embodiment 14 or 15, wherein the distance of the one or more detectable moieties comprises at least about 5 angstroms.
17. The detectable labeling reagent of any one of embodiments 1-16, wherein the backbone unit comprises an oligopeptide according to G-K-X₁-X₂-K-G, wherein:
   X₁ is selected from the group consisting of A, E, N, C, P, D, S, R, M, L, G, N, and H; and
   X₂ is selected from the group consisting of S, P, I, E, L, V, P, H, T, V, Q, and R.
18. The detectable labeling reagent of embodiments 1-17, wherein the backbone unit comprises X₁-X₂ according to:
   AS, AP, EP, NI, CE, PS, DL, SV, RP, RH, DT, MV, DQ, LS, GV, NQ DI, HT, or RR.
19. The detectable labeling reagent of any one of embodiments 1-18, wherein the backbone unit substantially constrains an average deviation of the one or more detectable moieties comprises relative to the chemical handle.
20. The detectable labeling reagent of embodiment 19, wherein the average deviation comprises at most about 5 angstroms.
21. The detectable labeling reagent of embodiment 19 or 20, wherein the average deviation comprises at least about 1 angstrom.
22. The detectable labeling reagent of any one of embodiments 19-21, wherein the backbone unit comprises an oligopeptide according to X₃-X₁-X₂-X₄, wherein:
   X₁ is selected from the group consisting of G, N, I, L, Q, A, D, C, S, and R; and
   X₂ is selected from the group consisting of I, Q, G, V, S, T, P, and L.
23. The detectable labeling reagent of any one of embodiments 19-22, wherein the backbone unit comprises an oligopeptide according to X₃-X₁-X₂-X₄, wherein:
X₁-X₂ is according to GI, NQ, GG, IV, LS, QQ, AT, QV, DI, CQ, QG, SS, GT, RV, NP, NL, AL, QT, or GV.
24. The detectable labeling reagent of any one of embodiments 1-23, wherein the conformation of the backbone unit substantially constrains an orientation of the one or more detectable moieties relative to the chemical handle.
25. The detectable labeling reagent of any one of embodiments 1-24, wherein the orientation of the one or more detectable moieties comprises an average angular deviation of at most about 60 degrees relative to the chemical handle.
26. The detectable labeling reagent of any one of embodiments 1-25, wherein the orientation of the one or more detectable moieties comprises an average angular deviation of at most about 45 degrees relative to the chemical handle.
27. The detectable labeling reagent of any one of embodiments 1-26, wherein the orientation of the one or more detectable moieties comprises an average angular deviation of at most about 30 degrees relative to the chemical handle.
28. The detectable labeling reagent of any one of embodiments 1-27, wherein the conformation of the backbone unit substantially constrains a position of the one or more detectable moieties relative to the chemical handle.
29. The detectable labeling reagent of any one of embodiments 1-28, wherein the position of the one or more detectable moieties comprises an average deviation of at most about 10 nanometers (nm) relative to the chemical handle.
30. The detectable labeling reagent of any one of embodiments 1-29, wherein the position of the one or more detectable moieties comprises an average deviation of at most about 6 nanometer (nm) relative to the chemical handle.
31. The detectable labeling reagent of any one of embodiments 1-30, wherein the position of the one or more detectable moieties comprises an average deviation of at most about 4 nanometers (nm) relative to the chemical handle.
32. The detectable labeling reagent of any one of embodiments 1-31, wherein the position or the orientation of the one or more detectable moieties is an average position relative to the chemical handle.
33. The detectable labeling reagent of any one of embodiments 1-32, wherein the position of the one or more detectable moieties is at least 1 nanometer (nm) from the chemical handle.
34. The detectable labeling reagent of any one of embodiments 1-33, wherein the position of the one or more detectable moieties is at least 2 nanometers (nm) from the chemical handle.
35. The detectable labeling reagent of any one of embodiments 1-34, wherein the position of the one or more detectable moieties is at least 5 nanometers (nm) from the chemical handle.
36. The detectable labeling reagent of any one of embodiments 1-35, wherein the backbone unit comprises the conformation when coupled to the target biomolecule.
37. The detectable labeling reagent of any one of embodiments 1-36, wherein the conformation comprises stability at 60° C.
38. The detectable labeling reagent of any one of embodiments 1-37, wherein the backbone unit comprises a peptide.
39. The detectable labeling reagent of embodiment 38, wherein the peptide comprises between 4 and 20 amino acids.
40. The detectable labeling reagent of either of embodiments 38 or 39, wherein the peptide comprises between 6 and 15 amino acids.
41. The detectable labeling reagent of any one of embodiments 38-40, wherein the peptide comprises between 8 and 12 amino acids.
42. The detectable labeling reagent of any one of embodiments 38-41, wherein the peptide comprises at least 3 unique amino acids.
43. The detectable labeling reagent of any one of embodiments 38-42, wherein the peptide comprises at least 5 unique amino acids.
44. The detectable labeling reagent of any one of embodiments 38-43, wherein the peptide comprises a non-natural amino acid.
45. The detectable labeling reagent of any one of embodiments 38-44, wherein the peptide comprises a non-amino acid moiety.
46. The detectable labeling reagent of embodiment 45, wherein the non-amino acid moiety is coupled to at least two amino acids of the oligopeptide backbone unit.
47. The detectable labeling reagent of any one of embodiments 38-46, wherein the conformation comprises a secondary structural feature of the peptide.
48. The detectable labeling reagent of embodiment 47, wherein the secondary structural feature comprises an alpha-helix.
49. The detectable labeling reagent of any one of embodiments 38-48, wherein the peptide comprises a disulfide bond.
50. The detectable labeling reagent of any one of embodiments 1-49, wherein the backbone unit comprises a second conformation, and wherein the backbone unit is configured to interconvert between the conformation and the second conformation.
51. The detectable labeling reagent of embodiment 50, wherein an intensity of a signal of the one or more detectable moieties is greater when the backbone unit comprises the conformation than when the backbone unit comprises the second conformation.
52. The detectable labeling reagent of either of embodiments 50 or 51, wherein a wavelength of a signal of the one or more detectable moieties changes when the backbone unit converts between the conformation and the second conformation.
53. The detectable labeling reagent of any one of embodiments 50-52, wherein a wavelength of a signal of the one or more detectable moieties is greater when the backbone unit comprises the conformation than when the backbone unit comprises the second conformation.
54. The detectable labeling reagent of any one of embodiments 50-53, wherein interconversion of the backbone unit between the conformation and the second conformation is light mediated.
55. The detectable labeling reagent of any one of embodiments 50-54, wherein interconversion of the backbone unit between the conformation and the second conformation is temperature mediated.
56. The detectable labeling reagent of any one of embodiments 50-55, wherein interconversion of the backbone unit between the conformation and the second conformation is chemically mediated.
57. The detectable labeling reagent of any one of embodiments 50-56, wherein interconversion of the backbone unit between the conformation and the second conformation is pH mediated.
58. The detectable labeling reagent of any one of embodiments 1-57, wherein the backbone unit is linear.
59. The detectable labeling reagent of any one of embodiments 1-58, wherein the backbone unit is branched.
60. The detectable labeling reagent of any one of embodiments 1-59, wherein the backbone unit comprises a cyclic or polycyclic structure.
61. The detectable labeling reagent of any one of embodiments 1-60, wherein the chemical handle is inert towards the backbone unit and the one or more detectable moieties.
62. The detectable labeling reagent of any one of embodiments 1-61, wherein the chemical handle is configured to selectively couple to an amino acid type.
63. The detectable labeling reagent of embodiment 62, wherein the amino acid type comprises cysteine, lysine, tyrosine, histidine, aspartic acid, glutamic acid, tryptophan, arginine, methionine, or any combination thereof.
64. The detectable labeling reagent of either of embodiments 62 or 63, wherein the amino acid type comprises cysteine, and wherein the chemical handle comprises an iodoacetamide, a thiol, a benzyl halide, an allyl halide, a selenocyanate, a maleimide, an alkyne, or any combination thereof.
65. The detectable labeling reagent of any one of embodiments 62-64, wherein the amino acid type comprises lysine, and wherein the chemical handle comprises a thiocyanate, an isothiocyanate, a maleimide, an aldehyde, an isatoic anhydride, an NHS ester, or any combination thereof.
66. The detectable labeling reagent of any one of embodiments 62-65, wherein the amino acid type comprises aspartic acid or glutamic acid, and wherein the chemical handle comprises an amine, an alcohol, a thiol, an organocuprate, or any combination thereof.
67. The detectable labeling reagent of any one of embodiments 62-66, wherein the amino acid type comprises tyrosine, and wherein the chemical handle comprises a diazonium compound.
68. The detectable labeling reagent of any one of embodiments 62-67, wherein the amino acid type comprises histidine, and wherein the chemical handle comprises an alpha-beta unsaturated carbonyl compound, an epoxide, or a combination thereof.
69. The detectable labeling reagent of any one of embodiments 62-68, wherein the amino acid type comprises arginine, and wherein the chemical handle comprises an NHS ester.
70. The detectable labeling reagent of any one of embodiments 62-69, wherein the amino acid type comprises methionine, and wherein the chemical handle comprises an oxaziridine compound.
71. The detectable labeling reagent of any one of embodiments 62-70, wherein the amino acid type comprises tryptophan, and wherein the chemical handle comprises a diazopropanoate ester.
72. The detectable labeling reagent of any one of embodiments 62-71, wherein the amino acid type comprises a post-translationally modified amino acid type.
73. The detectable labeling reagent of embodiment 72, wherein the post-translationally modified amino acid type comprises phosphorylation, glycosylation, nitrosylation, citrullination, sulfenylation, trimethylation, or any combination thereof.
74. The detectable labeling reagent of either of embodiments 72 or 73, wherein the post-translationally modified amino acid type comprises phosphoserine, phosphotyrosine, phosphothreonine, or any combination thereof, and wherein the chemical handle comprises a disulfide.
75. The detectable labeling reagent of embodiments 62-74, wherein the amino acid type comprises an N-terminal amino acid or a C-terminal amino acid.
76. The detectable labeling reagent of any one of embodiments 1-75, wherein the chemical handle is configured to couple to form a single attachment to the biomolecule.
77. The detectable labeling reagent of any one of embodiments 1-76, wherein the chemical handle is configured to stoichiometrically couple to the biomolecule.
78. The detectable labeling reagent of any one of embodiments 1-77, further comprising a triplet state quencher.
79. The detectable labeling reagent of any one of embodiments 1-78, wherein the one or more detectable moieties of the detectable labeling reagent comprises a half-life which is at least twice as long as a half-life of the one or more detectable moieties provided without the labeling reagent.
80. The detectable labeling reagent of any one of embodiments 1-79, wherein the backbone unit increases a half-life of the one or more detectable moieties by at least 50%,
81. A system comprising a biopolymer comprising a plurality of subunits,
   wherein a first subunit of the plurality of subunits is coupled to a first detectable label, wherein a second subunit of the plurality of subunits is coupled to a second detectable label,
   wherein the first and the second detectable labels each comprise:
      (i) a chemical handle for the coupling to the first and the second subunits,
      (ii) a backbone unit, and
      (iii) one or more detectable moieties;
   wherein the chemical handle of the first label is no more than 25 Å from the chemical handle of the second label,
   and wherein a combined intensity of a detectable signal of the first detectable label and the second detectable label is greater than an intensity of either the first or the second detectable label taken alone.
82. The method of embodiment 81, wherein the first or the second detectable labels independently comprise a detectable labeling reagent according to any one of embodiments 1-80 coupled to the first subunit or the second subunit.
83. A detectable labeling reagent for coupling to a biomolecule, the detectable labeling reagent comprising:
   i) a backbone unit,
   ii) a first fluorophore,
   iii) a second fluorophore, and
   iv) a functional handle for coupling to the biomolecule;
      wherein the first fluorophore and the second fluorophore are separated by at most 30 Å, and
      wherein a fluorescence intensity of the detectable labeling reagent is greater than a fluorescence intensity of the first fluorophore in the absence of the second fluorophore and a fluorescence intensity of the second fluorophore in the absence of the first fluorophore.
84. The detectable labeling reagent of embodiment 83, wherein the detectable labeling reagent comprises a structure according to any one of embodiments 1-80.
85. The detectable labeling reagent of embodiment 83 or 84, wherein the backbone unit comprises an oligopeptide with about 4 to about 20 amino acids.
86. The detectable labeling reagent of any one of embodiments 83-85, wherein the backbone unit comprises between about 6 and about 15 amino acids.
87. The detectable labeling reagent of embodiment 86, wherein the backbone unit comprises between about 8 and about 12 amino acids.
88. The detectable labeling reagent of any one of embodiments 83-87, wherein the backbone unit comprises at least 4 types of amino acids.
89. The detectable labeling reagent of embodiment 88, wherein the oligopeptide backbone unit comprises at least 6 types of amino acids.
90. The detectable labeling reagent of embodiment 83, wherein the oligopeptide backbone unit comprises a secondary structural feature.
91. The detectable labeling reagent of embodiment 90, wherein the secondary structural feature is stable at 60° C. 92. The detectable labeling reagent of embodiment 90, wherein the secondary structural feature comprises an alpha-helix.
93. The detectable labeling reagent of embodiment 90, wherein the oligopeptide backbone unit prevents contact between:
   (1) the functional handle, and
   (2) the first and the second fluorophores.
94. The detectable labeling reagent of embodiment 90, wherein the oligopeptide backbone unit comprises a disulfide bond.
95. The detectable labeling reagent of embodiment 90, wherein the oligopeptide backbone unit is configured to interconvert between a first state and a second state, and wherein a fluorescence intensity of the first fluorophore or the second fluorophore differs between the first state and the second state of the oligopeptide backbone unit.
96. The detectable labeling reagent of embodiment 96, wherein the oligopeptide backbone unit comprises different conformations in the first state and the second state.
97. The detectable labeling reagent of embodiment 96, wherein the interconverting between the first state and the second state comprises chemical interconversion.
98. The detectable labeling reagent of embodiment 96, wherein the interconverting between the first state and the second state is temperature mediated, chemical condition mediated, or light mediated.
99. The detectable labeling reagent of embodiment 90, wherein the oligopeptide backbone unit is linear.
100. The detectable labeling reagent of embodiment 90, wherein the backbone unit is branched.
101. The detectable labeling reagent of embodiment 90, wherein the backbone unit comprises a non-natural amino acid.
102. The detectable labeling reagent of embodiment 90, wherein the backbone unit comprises a non-amino acid moiety.
103. The detectable labeling reagent of embodiment 90, wherein the non-amino acid moiety is coupled to at least two amino acids of the backbone unit.
104. The detectable labeling reagent of embodiment 90, wherein an absorbance maximum of the first fluorophore and an absorbance maximum of the second fluorophore are within 10 nm.
105. The detectable labeling reagent of embodiment 90, wherein an emission maximum of the first fluorophore and an emission maximum of the second fluorophore are within 10 nm.
106. The detectable labeling reagent of embodiment 90, wherein the first fluorophore is identical to the second fluorophore.
107. The detectable labeling reagent of embodiment 90, wherein the first fluorophore and the second fluorophore are separated by at most 20 Å.
108. The detectable labeling reagent of embodiment 107, wherein the first fluorophore and the second fluorophore are separated by at most 12 Å.
109. The detectable labeling reagent of embodiment 90, wherein a relative orientation of the first fluorophore and the second fluorophore is constrained by the backbone.
110. The detectable labeling reagent of embodiment 109, wherein a dipole moment of the first fluorophore is substantially aligned with a dipole moment of the second fluorophore.
111. The detectable labeling reagent of embodiment 109, wherein the first fluorophore is constrained from rotating relative to the second fluorophore.
112. The detectable labeling reagent of embodiment 83, wherein the functional handle is inert towards the oligopeptide unit, the first fluorophore, and the second fluorophore.
113. The detectable labeling reagent of embodiment 83, wherein the functional handle is configured to selectively couple to an amino acid type.
114. The detectable labeling reagent of embodiment 113, wherein the amino acid type comprises cysteine, lysine, tyrosine, histidine, aspartic acid, glutamic acid, tryptophan, arginine, methionine, or any combination thereof.
115. The labeling reagent of embodiment 114, wherein the amino acid type comprises cysteine, and wherein the chemical moiety comprises an iodoacetamide, a thiol, a benzyl halide, an allyl halide, a selenocyanate, a maleimide, an alkyne, or any combination thereof.
116. The detectable labeling reagent of embodiment 114, wherein the amino acid type comprises lysine, and wherein the chemical moiety comprises a thiocyanate, an isothiocyanate, a maleimide, an aldehyde, an isatoic anhydride, an NHS ester, or any combination thereof.
117. The detectable labeling reagent of embodiment 114, wherein the amino acid type comprises aspartic acid or glutamic acid, and wherein the chemical moiety comprises an amine, an alcohol, a thiol, an organocuprate, or any combination thereof.
118. The detectable labeling reagent of embodiment 114, wherein the amino acid type comprises tyrosine, and wherein the chemical moiety comprises a diazonium compound.
119. The detectable labeling reagent of embodiment 114, wherein the amino acid type comprises histidine, and wherein the chemical moiety comprises an alpha-beta unsaturated carbonyl compound, an epoxide, or a combination thereof.
120. The detectable labeling reagent of embodiment 114, wherein the amino acid type comprises arginine, and wherein the chemical moiety comprises an NHS ester.
121. The detectable labeling reagent of embodiment 114, wherein the amino acid type comprises methionine, and wherein the chemical moiety comprises an oxaziridine compound.
122. The detectable labeling reagent of embodiment 114, wherein the amino acid type comprises tryptophan, and wherein the chemical moiety comprises a diazopropanoate ester.
123. The detectable labeling reagent of embodiment 113, wherein the amino acid type comprises a post-translationally modified amino acid type.
124. The detectable labeling reagent of embodiment 123, wherein the post-translationally modified amino acid type comprises phosphorylation, glycosylation, nitrosylation, citrullination, sulfenylation, trimethylation, or any combination thereof.
125. The detectable labeling reagent of embodiment 124, wherein the post-translationally modified amino acid type comprises phosphoserine, phosphotyrosine, phosphothreonine, or any combination thereof, and wherein the chemical moiety comprises a disulfide.
126. The detectable labeling reagent of embodiment 113, wherein the amino acid type comprises an N-terminal amino acid or a C-terminal amino acid.
127. The detectable labeling reagent of embodiment 83, wherein the functional handle is configured to couple to form a single attachment to the biomolecule.
128. The detectable labeling reagent of embodiment 83, wherein the functional handle is configured to stoichiometrically couple to the biomolecule.
129. The detectable labeling reagent of embodiment 83, further comprising a triplet state quencher.
130. The detectable labeling reagent of embodiment 83, wherein the first fluorophore or the second fluorophore comprises a plurality of fluorophores.
131. The detectable labeling reagent of embodiment 83, wherein the first fluorophore and the second fluorophore each comprise a plurality of fluorophores.
132. The detectable labeling reagent of embodiment 83, wherein the detectable labeling reagent is inert towards Edman degradation.
133. The detectable labeling reagent of embodiment 83, wherein the detectable labeling reagent comprises an aqueous solubility of at least 2 mg/mL.
134. The detectable labeling reagent of embodiment 133, wherein the detectable labeling reagent comprises an aqueous solubility of at least 20 mg/mL.
135. The detectable labeling reagent of embodiment 83, wherein the first fluorophore comprises a diminished rate of photobleaching than the first fluorophore in the absence of the backbone unit, the second fluorophore, and the functional handle.
136. A system comprising a peptide, wherein the peptide comprises an amino acid type, and wherein each of a plurality of amino acids of the amino acid type are coupled a detectable labeling reagent of any one of embodiments 1-80 or 83-135.
137. The system of embodiment 136, wherein the peptide is immobilized to a substrate.
138. The system of embodiment 137, wherein the substrate comprises a bead, a polymer matrix, a surface, or a slide.
139. The system of embodiment 136, wherein a C-terminus of the peptide is coupled to the substrate.
140. The system of embodiment 136, further comprising a dye repellant.
141. The system of embodiment 140, wherein a distance between the first fluorophore and the second fluorophore is greater than the distance in the absence of the dye-repellant.
142. The system of embodiment 140, wherein the dye repellant comprises a counterion for the first fluorophore or the second fluorophore.
143. The system of embodiment 140, wherein the dye repellant comprises an anion.
144. The system of embodiment 143, wherein the dye repellant comprises a dicarboxylic acid compound.
145. The system of embodiment 144, wherein the dicarboxylic acid compound comprises a benzenedicarboxylic acid.
146. The system of embodiment 136, wherein a conformation of the peptide is substantially similar to a conformation of the peptide in the absence of the optically detectable labeling reagent.
147. The system of embodiment 136, wherein a solubility of the peptide is similar to or greater than a solubility of the peptide in an absence of the optically detectable labeling reagent.
148. The system of embodiment 136, wherein an intensity of a fluorescence signal of the detectable labeling reagent is substantially linear with respect to the number of the plurality of amino acids coupled to the optically detectable labeling reagent.
149. The system of embodiment 136, wherein two amino acids of the plurality of amino acids coupled to the detectable labeling reagent are adjacent with respect to a sequence of the peptide.
150. The system of embodiment 136, wherein an intensity of a fluorescence signal of the detectable labeling reagent coupled to the two amino acids is approximately twice that of the detectable labeling reagent in a dilute solution.
151. The system of embodiment 136, wherein the peptide comprises a second amino acid type, and wherein a plurality of amino acids of the second amino acid type are coupled to a second detectable labeling reagent of any one of embodiments 1-52.
152. The system of embodiment 151, wherein the first fluorophore and the second fluorophore of the detectable labeling reagent are different than the first fluorophore and the second fluorophore of the second optically detectable labeling reagent.
153. The system of embodiment 152, wherein the detectable labeling reagent and the second detectable labeling reagent generate different fluorescence signals.
154. A method for analyzing a peptide, the method comprising:
   (a) coupling a detectable labeling reagent to an amino acid of the peptide, wherein the labeling reagent comprises a backbone unit, a plurality of fluorophores, and a functional handle for the coupling to the amino acid of the peptide;
   (b) detecting a signal from the labeling reagent coupled to the peptide; and
   (c) using the signal to identify the amino acid of the peptide.
155. The method of embodiment 154, wherein the detectable labeling reagent comprises a reagent according to any one of embodiments 1-80 or 83-135.
156. The method of embodiment 154, wherein the functional handle comprises specificity for the amino acid of the peptide.
157. The method of embodiment 154, wherein the amino acid comprises cysteine, lysine, tyrosine, histidine, aspartic acid, glutamic acid, tryptophan, arginine, methionine, N-terminal amino acids, C-terminal amino acids, or any combination thereof.
158. The method of embodiment 157, wherein the amino acid comprises N-terminal amino acids or C-terminal amino acids.
159. The method of embodiment 158, wherein the functional handle comprises pyridinecarboxyaldehyde (PCA).
160. The method of embodiment 154, wherein the labeling reagent couples to the peptide with about 1:1 stoichiometry.
161. The method of embodiment 154, wherein (c) further comprises quantifying a concentration or abundance of the peptide.
162. The method of embodiment 161, wherein the concentration of the peptide is less than about 1 pM.
163. The method of embodiment 154, wherein the amino acid comprises a plurality of amino acids, and wherein the labeling reagent couples to at least a subset of the plurality of amino acids.
164. The method of embodiment 163, wherein the labeling reagent couples to each of the plurality of amino acids.
165. The method of embodiment 164, wherein (c) comprises quantifying the plurality of amino acids of the peptide.
166. The method of embodiment 154, further comprising (d) cleaving at least a portion of the peptide, and (e) detecting a signal or a signal change from the labeling reagent coupled to the peptide.
167. The method of embodiment 166, further comprising (f) identifying a sequence of the peptide based at least in part on the signal or the signal change in (e).
168. The method of embodiment 154, wherein the detecting comprises imaging the labeling reagent coupled to the peptide.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structural features within the scope of these claims and their equivalents be covered thereby.

1. A detectable labeling reagent for coupling to a target biomolecule, said detectable labeling reagent comprising:
(i) a chemical handle configured to couple to said target biomolecule,
(ii) a backbone unit, and
(iii) one or more detectable moieties;
wherein said backbone unit comprises a conformation so that, when said detectable labeling reagent is coupled to said target biomolecule, said backbone unit substantially constrains a position or an orientation of a detectable moiety of said one or more detectable moieties relative to another detectable moiety of said one or more detectable moieties that is coupled to said target biomolecule.

2. The detectable label of claim 0, wherein said chemical handle is configured to couple to an amino acid of said target biomolecule.

3. (canceled)

4. The detectable label of claim 1 Error! Reference source not found., wherein said chemical handle comprises a primary amine-reactive group, a primary thiol-reactive group, a thioether-reactive group, a primary alcohol-reactive group, a phenol-reactive group, a carboxylic acid-reactive group, or a hydroxyl-reactive group, or any combination thereof.

5. The detectable label of claim 1, wherein said detectable labeling reagent further comprises a structure according to 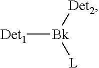
wherein Bk denotes said backbone unit;
Det₁ and Det₂ independently denote a first and a second detectable moiety; and
L denotes said chemical handle.

6. The detectable labeling reagent of claim 1, wherein said backbone unit further comprises an oligopeptide structure according to:
X₃-X₁-X₂-X₄ wherein;
X₃ and X₄ independently denote an amino acid side chain linked to a detectable moiety; and
X₁ and X₂ independently denote any amino acid.

7. The detectable labeling reagent of claim 1, wherein the backbone unit further comprises an oligopeptide structure according to:
X₃-X₁-X₂-X₄ wherein;
X₃ comprises 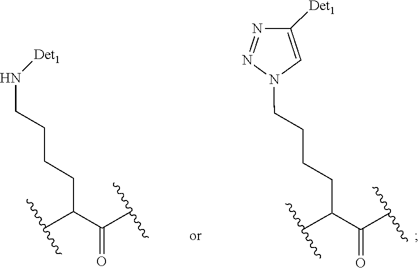
X₄ comprises 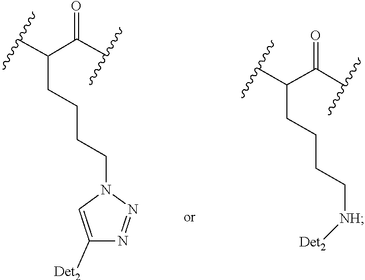
Det₁ and Det₂ independently denote first and second detectable moieties; and
X₁ and X₂ independently denote any amino acid.

8. (canceled)

9. The detectable labeling reagent of claim 1, wherein said backbone unit substantially constrains an orientation of said first and said second detectable moiety relative to each other.

10.-13. (canceled)

14. The detectable labeling reagent of claim 1 Error! Reference source not found., wherein said backbone unit substantially constrains a distance of said first and said second detectable moiety relative to each other.

15.-23. (canceled)

24. The detectable labeling reagent of claim 1 Error! Reference source not found., wherein said conformation of said backbone unit substantially constrains an orientation of said one or more detectable moieties relative to said chemical handle.

25.-27. (canceled)

28. The detectable labeling reagent of claim 1 Error! Reference source not found., wherein said conformation of said backbone unit substantially constrains a position of said one or more detectable moieties relative to said chemical handle.

29.-37. (canceled)

38. The detectable labeling reagent of claim 1 Error! Reference source not found., wherein said backbone unit comprises a peptide.

39.-49. (canceled)

50. The detectable labeling reagent of claim 1 Error! Reference source not found., wherein said backbone unit comprises a second conformation, and wherein said backbone unit is configured to interconvert between said conformation and said second conformation.

51.-61. (canceled)

62. The detectable labeling reagent of claim 1 Error! Reference source not found., wherein said chemical handle is configured to selectively couple to an amino acid type.

63. The detectable labeling reagent of claim 0, wherein said amino acid type comprises cysteine, lysine, tyrosine, histidine, aspartic acid, glutamic acid, tryptophan, arginine, methionine, or any combination thereof.

64.-71. (canceled)

72. The detectable labeling reagent of claim 62 Error! Reference source not found., wherein said amino acid type comprises a post-translationally modified amino acid type.

73.-74. (canceled)

75. The detectable labeling reagent of claim 62 Error! Reference source not found., wherein said amino acid type comprises an N-terminal amino acid or a C-terminal amino acid.

76.-80. (canceled)

81. A method for analyzing a peptide, said method comprising:
(a) coupling a detectable labeling reagent to an amino acid of said peptide, wherein said labeling reagent comprises a backbone unit, a plurality of fluorophores, and a functional handle for said coupling to said amino acid of said peptide; and
(b) detecting a signal from said labeling reagent coupled to said peptide; and
(c) using said signal to identify said amino acid of said peptide.

82.-89. (canceled)

90. The method of claim 81, wherein said amino acid comprises a plurality of amino acids, and wherein said labeling reagent couples to at least a subset of said plurality of amino acids.

91-92. (canceled)

93. The method of claim 81, further comprising (d) cleaving at least a portion of said peptide, and (e) detecting a signal or a signal change from said labeling reagent coupled to said peptide.

94. The method of claim 93, further comprising (f) identifying a sequence of said peptide based at least in part on said signal or said signal change in (e).

95. (canceled)
